SEQUENCE LISTING (3 sequences)

SEQ ID NO: 1 - DNA, 58 bp, Artificial Sequence
  Synthetic Construct
ccggacggctgaagaacgctatttcctcgaggaaatagcgttcttcagccgttttttg

SEQ ID NO: 2 - DNA, 57 bp, Artificial Sequence
  Synthetic Construct
ccgggcgatcacataaagcattatactcgagtataatgctttatgtgatcgcttttt

SEQ ID NO: 3 - DNA, 57 bp, Artificial Sequence
  Synthetic Construct
ccggcggcactacatggaagtgaatctcgagattcacttccatgtagtgccgttttt

(19) United States
(12) Patent Application Publication
(10) Pub. No.: US 2024/0182893 A1
(43) Pub. Date: Jun. 6, 2024

(54) INHIBITION OF FGR REDUCES FIBROSIS

(71) Applicant: UNIVERSITY OF PITTSBURGH-OF THE COMMONWEALTH SYSTEM OF HIGHER EDUCATION, Pittsburgh, PA (US)

(72) Inventors: Amitava MUKHERJEE, Pittsburgh, PA (US); Michael EPPERLY, Pittsburgh, PA (US); Joel GREENBERGER, Sewickley, PA (US)

(21) Appl. No.: 18/284,966

(22) PCT Filed: Apr. 1, 2022
(86) PCT No.: PCT/US2022/023050

§ 371 Date: Sep. 29, 2023

Related U.S. Application Data
(60) Provisional application No. 63/170,140, filed on Apr. 2, 2021.
(60) Provisional application No. 63/280,750, filed on Nov. 18, 2021.

(51) Int. Cl. C12N 15/113; A61K 31/337; A61K 31/517; A61N 5/10; A61P 11/00; G01N 33/50

(52) U.S. Cl. CPC C12N 15/113; A61K 31/337; A61K 31/517; A61N 5/10; A61P 11/00; G01N 33/5023; C12N 2310/14; G01N 2333/9121; G01N 2333/938

(57) ABSTRACT

Disclosed herein are compositions and methods for treating and preventing a disease or condition characterized by aberrant fibroblast proliferation and extracellular matrix deposition in a tissue of a subject in need thereof. The particular disease or condition can be fibrosis, specifically, radiation-induced fibrosis. The method for treating or preventing the disease or condition can include administering to the subject an effective amount of a composition that inhibits Fgr, a non-receptor tyrosine kinase.

### CROSS-REFERENCE TO RELATED APPLICATIONS

This application claims the benefit of priority to U.S. Provisional Application No. 63/170,140 filed Apr. 2, 2021 and U.S. Provisional Application No. 63/280,750 filed Nov. 18, 2021, both of which are hereby incorporated herein by reference in their entireties.

### STATEMENT REGARDING FEDERALLY SPONSORED RESEARCH

This invention was made with government support under grant number AI068021 awarded by the National Institutes of Health. The government has certain rights in the invention.

### BACKGROUND

Treatment of tumors by radiation therapy creates a dilemma common to many cancer treatments. Often in radiation therapy, there are side effects, many of which are dependent on the area of the body that is irradiated. A side effect seen with ionizing radiation is radiation-induced fibrosis in the area of the body that was irradiated. Radiation-induced fibrosis remains the most important dose-limiting toxicity of radiation therapy to soft tissue. Radiation-induced fibrosis can develop as a late effect of radiation therapy in skin and subcutaneous tissue, lungs, the gastrointestinal and genitourinary tracts, muscles, or other organs, depending upon the treatment site. There have been many theories, but few to no therapies, for treatment or prevention of radiation-induced fibrosis. The prevention of radiation-induced fibrosis has focused on improvements in radiation techniques, which have resulted in higher doses to the tumor target and decreased doses to normal tissue, thus limiting the development of radiation-induced fibrosis.

Many other disease processes, including cardiomyopathies, hypertension, chronic hepatitis C infection, adult respiratory distress syndrome, and sarcoidosis are accompanied by fibrosis. Tissue fibrosis is characterized by abnormal fibroblast proliferation and migration, and accumulation of extracellular matrix, thought to arise from unresolved tissue repair. Fibrosis can affect many organ systems including, among others, the lung, kidney, liver and heart.

There remains a need for pharmacological methods and compositions that can effectively control the development and progression of fibrotic disease. In one particular example, there is a need for methods and compositions that are provided to patients who are undergoing or have undergone radiation treatments for the prevention and treatment of radiation-induced fibrosis. The disclosed compounds, compositions, and methods are directed to overcoming these and other needs in the art.

### SUMMARY

Fibrosis processes are driven by a cascade of injury, inflammation, fibroblast proliferation and migration, and matrix deposition and remodeling. Senescence cells have been implicated in pulmonary fibrosis tissue and senescent cell deletion has been shown to rejuvenate pulmonary health in aged mice. The molecular mechanism by which senescent cells regulate fibrosis is unknown. Herein, a tyrosine kinase, Fgr, is identified by RNA-seq analysis of sorted tdTOMp16+ mouse radiation-induced senescence (RIS) cells, which is significantly upregulated to a level greater than that of any other transcript including those associated with chemotaxis or phagocytosis. Pharmacological compounds or methods known to inhibit or silence Fgr have been shown to be potent inhibitors of profibrotic genes.

In accordance with the purposes of the disclosed materials and methods, as embodied and broadly described herein, the disclosed subject matter, in one aspect, relates to compounds, compositions and methods for treating and preventing a disease or condition characterized by aberrant fibroblast proliferation and extracellular matrix deposition in a tissue of a subject in need thereof. The method for treating or preventing the disease or condition can include administering to the subject an effective amount of a composition that inhibits Fgr. As described herein, Fgr is a non-receptor tyrosine kinase belonging to the Src family kinases. The disease or condition characterized by aberrant fibroblast proliferation and extracellular matrix deposition in a tissue can result from idiopathic pulmonary fibrosis (IPF), pneumonia, acute respiratory distress syndrome (ARDS), asbestosis, bleomycin exposure, silicosis, anthracosis, post bacterial infectious fibrosis, viral (including Covid-19) liver fibrosis, post heat burn fibrosis, post ultraviolet light fibrosis, post trauma fibrosis, myocardial infarction, injury related tissue scarring, scarring form surgery, radiation exposure, allergic reaction, inhalation of environmental particulates, smoking, infection, mechanical damage, transplantation, autoimmune disorder, genetic disorder, a disease condition (such as scleroderma lung disease, rheumatoid arthritis, sarcoidosis, tuberculosis, Hermansky Pudlak Syndrome, bagassosis, systemic lupus erythematosis, eosinophilic granuloma, Wegener's granulomatosis, lymphangioleiomyomatosis, or cystic fibrosis), nitrofurantoin exposure, amiodarone exposure, cyclophosphamide exposure, methotrexate exposure, or a combination thereof.

In some examples, the disease or condition characterized by aberrant fibroblast proliferation and extracellular matrix deposition in a tissue is fibrosis. The fibrosis disease or condition can be idiopathic pulmonary fibrosis (IPF), asbestosis, silicosis, anthracosis, post bacterial infectious fibrosis, viral (including Covid-19) liver fibrosis, post heat burn fibrosis, post ultraviolet light fibrosis, post trauma fibrosis, scleroderma, scarring, liver fibrosis, kidney fibrosis, gut fibrosis, radiation-induced fibrosis, bleomycin-induced fibrosis, asbestos-induced fibrosis, biliary duct injury-induced fibrosis, head and neck fibrosis, burn induced fibrosis, surgical fibrosis, spinal cord fibrosis, or lung fibrosis. In specific examples, the fibrosis is lung fibrosis, such as radiation-induced fibrosis (e.g., ionizing radiation induced fibrosis).

Accordingly, provided herein are methods for treating or preventing radiation-induced fibrosis in a subject in need thereof. The method for treating or preventing radiation-induced fibrosis can include administering to the subject an effective amount of a composition that inhibits Fgr (a non-receptor tyrosine kinase). The radiation-induced fibrosis can be an ionizing radiation-induced fibrosis, such as radiation therapy for cancer treatment. The composition that inhibits Fgr can be administered to the subject prior to, during, or after exposure of the subject to ionizing radiation, or a combination thereof.

In other aspects, methods for preventing fibrosis or a disease or condition mediated by Fgr in a subject exposed to ionizing radiation are disclosed. The method for preventing fibrosis or a disease or condition mediated by Fgr can include administering to the subject an effective amount of a composition that inhibits Fgr, and irradiating the subject with ionizing radiation.

In further aspects, methods for treating or preventing a disease or condition characterized by aberrant fibroblast proliferation and extracellular matrix deposition in a tissue of a subject in need thereof, wherein the disease or condition is mediated by Fgr, are disclosed. The method for treating or preventing a disease or condition mediated by Fgr can include, administering to the subject an effective amount of a composition that inhibits Fgr. In some examples, the disease or condition is radiation-induced fibrosis, such as ionizing radiation induced fibrosis.

The compositions described herein that inhibit Fgr can include a pharmacological compound that inhibits tyrosine kinase. For example, the compositions can include a N-phenylbenzamide tyrosine kinase inhibitor, such as TL02-59. In other examples, the compositions that inhibit Fgr can comprise shRNA that interferes with Fgr expression; CRISPR for editing or knockout of Fgr; siRNA, antisense RNA, or nucleic acids that interfere with or prevent the transcription or translation of Fgr genes; antisense molecules comprising Fgr sequences; active sites that bind at least a portion of Fgr; interfering peptides; interfering nucleic acids; or a combination thereof. In some examples, the compositions that inhibit Fgr can include shRNA that interferes with Fgr expression. Specifically, shRNA mediated knockdown of Fgr can inhibit induction of radiation fibrosis in target cells. In some examples, the composition that inhibits Fgr can comprise MMS350.

In an exemplary embodiment, the compounds, compositions, and methods disclosed herein are for treating or preventing radiation induced pulmonary fibrosis (RIPF). The data herein using RNA-seq analysis with a pure population of sorted RIS cells expressing the tdTOMp16+ reporter gene, show that there is significant upregulation of the unique p15 tyrosine kinase. Fgr, in RIS cells. The degree of upregulation (seventy two-fold) is greater than that of any other gene including those involved in chemotaxis or phagocytosis. The data with a transwell co-culture system show that RIS cells induce biomarkers of fibrosis including collagen 1a, collagen 3, and TGF-β in separated target cells. The small molecule inhibitor of Fgr, TL02-59, was shown to abrogate the induction of biomarkers of fibrosis by RIS cells. The data also show that Fgr is detected in senescent cells in irradiated mouse lung in situ significantly before the appearance of fibrosis. There is currently no report of Fgr involvement in RIPF. Other tyrosine kinases (PDGF receptor, VEGF receptor, EGF receptor, and JAK kinases) as well as non-receptor tyrosine kinases (c-Abl, c-Kit, and Src kinases) are not increased in RIS cells.

Administering the composition as described herein can be carried out orally, parenterally, periadventitially, subcutaneously, intravenously, intramuscularly, intraperitoneally, by inhalation, by intranasal instillation, by implantation, by intracavitary or intravesical instillation, intraocularly, intraarterially, intralesionally, transdermally, intradermally or by application to mucous membranes. In the methods described herein, one or more additional anti-fibrotic agent can be administered to the subject. The additional anti-fibrotic agent can be selected from the group consisting of calcium channel blockers, cytotoxic agents, cytokines, chemokines, integrins, growth factors, hormones, lysophosphatidic acid (LPA) receptor 1 antagonists, agents that modulate the TGF-β pathway, endothelin receptor antagonists, agents that reduce connective tissue growth factor (CTGF) activity, matrix metalloproteinase (MMP) inhibitors, agents that reduce the activity of platelet-derived growth factor (PDGF), agents that interfere with integrin function, agents that interfere with the pro-fibrotic activities of cytokines, agents that reduce oxidative stress, PDE4 inhibitors, PDE5 inhibitors, mTOR inhibitors, modifiers of the arachidonic acid pathway, peroxisome proliferator-activated receptor (PPAR)-γ agonists, kinase inhibitors, inhibitors of VEGF signaling pathway, matrix metalloproteinases, tissue inhibitors of metalloproteinases (TIMPs), HGF agonists, angiotensin-converting enzyme (ACE) inhibitors, angiotensin receptor antagonists, inhibitors of advanced glycation endproducts (AGEs) or their receptors (RAGEs), Rho kinase inhibitors, PKC inhibitors, ADAM-10 inhibitor, famesoid X receptor agonists, caspase inhibitors, anti-oxidants, inhibitors of collagen expression, LMW heparin or heparin analogs, copper chelators, TNF-α blocking agents, HMG-CoA reductase inhibitors, and Thy-1 (CD90) inhibitors. The subject in need thereof can be human.

Diagnostic methods for identifying a pharmacological compound for treating or preventing fibrosis in a subject in need thereof are also disclosed. The methods can include obtaining a population of p16 or β-galactosidase (SA-β-gal) expressing senescent cells from the subject, identifying Fgr positive senescent cells, contacting the Fgr positive senescent cells with the pharmacological compound, and determining whether the pharmacological compound inhibits Fgr in the Fgr positive senescent cells. The diagnostic methods can also be used for identifying a pharmacological compound for treating or preventing radiation induced fibrosis in a subject in need thereof. For example, the methods can include obtaining a population of radiation induced p16 or β-galactosidase (SA-β-gal) expressing senescent cells from the subject; identifying Fgr positive senescent cells, contacting the Fgr positive senescent cells with the pharmacological compound, and determining whether the pharmacological compound inhibits Fgr in the Fgr positive senescent cells.

In some embodiments, the population of radiation induced p16 or β-galactosidase (SA-β-gal) expressing senescent cells can be pure or substantially pure, such as at least 80% purity, at least 85% purity, at least 90r % purity, or at least 95% purity. In some embodiments, obtaining a population of radiation induced p16 or β-galactosidase (SA-R-gal) expressing senescent cells is by fluorescence activated cell sorting (FACS). The method can further include isolating the Fgr positive senescent cells.

Pharmaceutical compositions for the treatment or prevention of fibrosis comprising a pharmaceutically acceptable excipient, a therapeutically effective amount of a N-phenylbenzamide tyrosine kinase inhibitor, such as TL02-59, and a propellant are also disclosed. The propellant can be selected from compressed air, ethanol, nitrogen, carbon dioxide, nitrous oxide, hydrofluoroalkanes (HFA), 1,1,1,2,-tetrafluoroethane, 1,1,1,2,3,3,3-heptafluoropropane, or combinations thereof. A pressurized container comprising the pharmaceutical compositions described herein are disclosed. The container can be a manual pump spray, inhaler, meter-dosed inhaler, dry powder inhaler, nebulizer, vibrating mesh nebulizer, jet nebulizer, or ultrasonic wave nebulizer.

### DESCRIPTION OF THE FIGURES

FIG. 1 shows selection of optimum cell density at the time of irradiation, radiation dose, and days to induce maximum % of red p16^{tdTom} cells before cell sorting. The figure shows at day 10, approximately 9% of cells turned red. It shows cells plated at 50% confluency at a dose of 5 Gy was the optimum condition. (n=4; *, p=<0.5; **, p=<0.01), p values were calculated by Two-way ANOVA. Significance shown only in 5 and 7.5 Gy for 9 and 10 days. Although 7.5 Gy produced as much red cells as 5 Gy, 5 Gy was chosen to irradiate the cells to reduce toxicity.

FIG. 2A confirms senescence of irradiated red tdTOMp16+ cells. FIG. 2A shows that irradiated red tdTOMp16+ cells do not divide over time. tdTOMp16+ non-irradiated cells (0 Gy) or irradiated (5 Gy) red, and irradiated non-red cells were a FACS sorted plated as single cells in 96-well plates. Single wells were then monitored over a period of 4-weeks. The graph shows % of cell division in irradiated red, irradiated non-red and non-irradiated cells. Briefly, after single cell sorting, 960 irradiated (5 Gy) td-TOM positive red cells were followed for 4 weeks. 0/960 cells divided. After 2 weeks, 137 of these cells remained intact. The red td-TOM cells were significantly larger than other control cells. In contrast 800 cells that were irradiated but did not turn red were followed, 50 of them divided. On the contrary 560 control cells (0 Gy) were followed and 104 of them divided. (n=8-10, **, p=0.01; ***, p=0.001, ***, p=0.0001; One-way ANOVA followed by Turkey's test).

FIG. 2B. Senescent markers p16 and p21 (not shown), and SA-β-Gal were significantly upregulated in senescent cells (**, p=0.01; ***, p=0.001; ***, p=0.0001; One-way ANOVA and t-test).

FIG. 3 shows principal component analysis (PCA) of RNA-seq data. RNA-seq was performed in triplicate with control p16tdTOM+ cells (0 Gy) and FACS sorted irradiated (5 Gy) p16tdTOM red and irradiated (5 Gy) p16tdTOM+ non-red cells 10 days after irradiation. PCA from RNA-seq data (9 samples, 12796 genes) shows distinct separation of the three groups.

FIG. 4A is a heatmap demonstrating clear differential gene expression of 1207 genes between irradiated red, irradiated non-red, and non-irradiated tdTOMp16+ cells. Red indicates upregulation, and blue, downregulation Color key indicates intensity associated to normalized expression values.

FIG. 4B. Volcano plot from the RNA-seq analysis of 5 Gy red vs. 5 Gy non-red cells shows differentially expressed (up and down regulated) genes (FIG. 4B).

FIG. 5A-FIG. 5C show validation of Fgr upregulation by RNA-seq with qPCR for: Fgr (FIG. 5A); p16 (FIG. 5B); and p21 (FIG. 5C). (n=3, **, p=0.01; ***, p=0.001). (one-way ANOVA followed by Tukey's test).

FIG. 6A-FIG. 6D show upregulation of profibrotic genes in target cells by senescent cells. In a noncontact transwell co-culture system (FIG. 6A) (Millipore filter separated) two experiments with FIG. 6B) top layer irradiated top layer unsorted tdTOMp16+ cells, and FIG. 6C) sorted irradiated red cells compared to irradiated non-red, there is significantly induction in target cells (bottom layer) of collagen 1a1 and collagen 3. Target C57BL/6 marrow stromal cell line. (n=2-3, p=<0.05; **, p=0.01 (*)) (NS=non-significant). FIG. 6D) Fgr inhibitor TL02-59 (1 μm) blocks senescent cell induction in target cells of collagen 3 in the non-contact transwell culture system after 2 weeks. (For FIG. 6B and FIG. 6C, two-sided two-sample t-test. FIG. 6D, p<0.001 for TL02-59, one-way ANOVA followed by Tukey's test).

FIG. 7A-FIG. 7B show shRNA knockdown of Fgr in vitro and inhibition of activation of Fgr by TL02-59. FIG. 7A) The tdTOMp16+ cell line was transfected with three individual Fgr shRNAs. Western blot shows significant knockdown of Fgr protein at 72 h after transfection by all three shRNAs. FIG. 7B) Inhibition of phosphorylation of Fgr in same cells by 0.1 μM TL02-59, which blocks its activation. (Phospho-FGR (Tyr412) Rabbit Polyclonal Antibody. Company: Thermo Fisher Scientific, Catalogue #PA5-64583).

FIG. 8A-FIG. 8B show irradiated mouse lungs demonstrate Fgr+ RIS cells before detectable fibrosis. tdTOMp16+ mice were irradiated to 20 Gy thoracic irradiation. Senescent cells detected by both red color, and p16 were merged with Fgr positive cells: (anti-Fgr, Santa Cruz Inc., Cat. #cc-74542) FIG. 8A) day 75 (no fibrosis) compared to day 150 (fibrosis). FIG. 8B) Quantitation of irradiation-induced senescent Fgr+ cells in lung prior to detection of fibrosis. Mean+SEM for 100 fields scored per lung lobe×5×5 mice per group. (One Way ANOVA followed by Tukey's test). *p<0.05 compared to Day 0 **p<0.001 compared to day 75.

FIG. 9A-FIG. 9C show upregulation of Fgr in radiation induced senescent human cell lines. FIG. 9A) Human bronchial epithelial cell line IB3, and FIG. 9B) Human bone marrow stromal cell line KM101 were irradiated (5 Gy) and stained with SA-beta-GAL staining kit and Fgr antibody. FIG. 9C) Graph represents quantitative analysis (%) for SA-beta-GAL and Fgr positive cells in IB3 (top) and KM101 (bottom) cell lines. The statistical analysis was done by non-parametric t-test. N=3, *, p<0.05; **, p<0.01.

FIG. 10 shows immunofluorescent staining of Fgr (Green) in human cell line RT4 shows localization to plasma membrane. Image obtained from Thul P J et al., A subcellular map of the human proteome. Science. (2017).

FIG. 11A-FIG. 11D show Fgr knockdown in irradiated tdTOMp16+ senescent cells reduces its profibrotic potential. Expression of Fgr was silenced by shRNAs and evaluated by qPCR (FIG. 11A), and Western blot (FIG. 11B). Both control and shRNA knockdown cells were sorted for irradiated red senescent and non-red cells and plated on the top well of transwells and cocultured with C57BL/6 stromal cells at the bottom (FIG. 11C). The expression of Collagen3 and TGFβ in the C57BL/6 stromal cells was evaluated by qPCR (FIG. 11D). N=3, * p<0.05; **, p<0.01; ***, p<0.001.

FIG. 12 shows robust upregulation of Fgr in idiopathic pulmonary fibrosis (IPF) lungs. Data obtained from IPF atlas.

FIG. 13 shows upregulation of Fgr in IPF monocytes, macrophages and in dendritic cells. Data obtained from IPF atlas.

FIG. 14. Upregulation of profibrotic genes in target cells by irradiated mouse lung senescent cells. In a noncontact transwell co-culture system, filter separated (19) sorted irradiated red and irradiated non-red mouse lung cells plated on top layer, there is significant induction of Tgf-β. Collagen 1a1 and collagen 3 in target cell line (bottom layer). Fgr inhibitor TL02-59 (10 nM) can block senescent cell induction of these genes in target cells (n=2-3, *, p=<0.05; **, p=<0.01, t-test).

FIG. 15A-FIG. 15B. Fgr shRNA knockdown in RIS stromal cells abrogates profibrotic gene expression in target cells in transwell. (FIG. 15A), shRNA knockdown of Fgr by western blot, (FIG. 15B) qPCR of Tgf-β and Collagen 3 in target cells. (n=2-3, *, p=<0.05; **, p=<0.01, t-test).

FIG. 16A-FIG. 16B. Irradiated mouse lungs show Fgr+ RIS cells before (day 75) and during (Day 150) detectable fibrosis. FIG. 16A) tdTOMp16+ mice were irradiated to 20 Gy thoracic radiation (Kalash R et al. Radiat Res. 2013, 180(5), 474-490). p16+ senescent cells were merged with Fgr+ cells. FIG. 16B) quantification of RIPF Fgr+ cells in mouse lungs. Mean+/−SEM from 10 images taken per group. (*, p=<0.05; **, p=<0.01, t-test, *, compared to day 0, **, compared to day 75) FIG. 17A-FIG. 17B. Fgr expression is in immune cells in RIPF lung cells by Single-cell RNA-Seq. FIG. 17A) shows different cell types identified from control and irradiated lungs FIG. 17B) scRNAseq analysis of Fgr expressing cell types from control and irradiated lungs shows upregulation of Fgr in neutrophils (p=1.6e-14), neutrophils (p=4e-11) and macrophages (one clusters, p=0.0025) (Hernandez-Segura A et al. Current Biology, 2017, 27, 2652-2660). (n=3 control and 3, 150 days post-irradiated (20 Gy) mice).

FIG. 18. Induction of Fgr and p16 in cells within human RIPF. Surgically resected lung of a radiotherapy patient at 24-months after 60 Gy treatment for lung cancer, and age matched control lungs were stained for Fgr, p16, α-SMA and DAPI (20×). Images are representative of 3 separate patient samples.

FIG. 19A-FIG. 19C. Optimization of in vitro radiation induction of senescent tdTOMp16+ cells. FIG. 19A—After 10 days, 5-Gy irradiated cells, plated at 50% confluency, yield ˜9% of red (tdTOM+) cells (n=4, *P=<0.5; **P=<0.01. The statistical significance was calculated using one-way ANOVA with multiple comparison). FIG. 19B—Irradiated (5 Gy) tdTOM+ and irradiated (5 Gy) tdTOM− cells were FACS sorted using gating relative to non-irradiated cells. FIG. 19C—PCA analysis from RNA-seq using three groups of cells (irradiated tdTOM+, irradiated tdTOM− and non-irradiated cells) show distinct separation in their gene expression and similarity of the three replicates (n=3). The transcriptomes of triplicate isolates of three groups of cells were sequenced: irradiated tdTOM+ senescent cells (0.32×106 cells each) replicate, irradiated tdTOM− non-senescent cells (2×106 cells), and nonirradiated cells (5×10⁶ cells).

FIG. 20A-FIG. 20C. Irradiation-induced tdTOMp16+ senescent cells display a unique transcriptome compared to non-irradiated, or irradiated, but non-senescent cells. FIG. 20A—The RNA-seq heat map illustrates the expression level of the DEGs between groups. Each row represents a gene and of the nine columns, from left to right, the first three are irradiated senescent (tdTOM+) samples, the next three columns in the middle are irradiated non-senescent (tdTOM−) samples, and the three columns on the right are non-irradiated samples. The value is the z-score of normalized gene expression counts. For any given gene, the red color represents gene expression that is greater than the overall mean, and the blue color represents gene expression that is less than the overall mean. Hierarchical clustering of genes and samples are represented by the dendrograms on the left and across the bottom of the heat map. FIG. 20B—Venn diagram depicting the shared and unique distribution of the DEGs. On the left are upregulated genes with comparisons between all three groups and on the right are comparisons with downregulated genes. Genes comparing irradiated senescent & irradiated non-senescent samples group are in black circle, irradiated senescent & nonirradiated samples are in red circle, and irradiated non-senescent & non-irradiated samples are in the green circle. FIG. 20C—Volcano plots show the log 2 (fold change) and −log 10 (adjusted P value) of the genes comparing irradiated senescent and irradiated non-senescent cells from the RNA-seq dataset. The statistically significant DEGs are represented in red. The statistical criteria for a gene to be considered differentially expressed was a fold change ≥2 and an adjusted P value≤0.05.

FIG. 21A-FIG. 21C. Prominent transcripts in senescent tdTOMp16+ cells include those, which are associated with fibrosis. FIG. 21A—The top 20 up- and downregulated genes in irradiated senescent & irradiated non-senescent samples. FIG. 21B—Top 20 up- and down-regulated genes in irradiated senescent relative to non-irradiated non-senescent samples. FIG. 21C—The heat map illustrates the expression level of the differentially expressed profibrotic genes between the three groups. Each row represents a gene and of the nine columns, from left to right, the first three (yellow bar) are irradiated senescent (tdTOM+) samples, the next three columns (green bar) in the middle are irradiated non-senescent (tdTOM−) samples, and the three columns on the right (black bar) are non-irradiated samples. The value is the z-score of normalized gene expression counts. For any given gene, the red color represents gene expression that is greater than the overall mean, and the blue color represents gene expression that is less than the overall mean. Hierarchical clustering of genes and samples is represented by the dendrograms on the left and across the bottom of the heat map.

FIG. 22A-FIG. 22D. Senescent tdTOMp16+ cells induce biomarkers of fibrosis in target cells. FIG. 22A—Scheme of non-contact transwell coculture system. The top well and the bottom well were separated by 0.4-micron filter. FIG. 22B—Irradiated tdTOMp16+ senescent (tdTOM+) and non-senescent (tdTOM−) cells were cultured on top well and the target C57BL/6 stromal cells were cultured on the bottom well. After 10 days, target cells were harvested for RNA and RT-qPCR was performed for Collagen1a1, Collagen 3, and TGF-β, CTGF, and α-smooth muscle actin (α-SMA) genes. FIG. 22C—Irradiated (5 Gy) and non-irradiated (0 Gy) tdTOMp16+ cells were cultured on top well and the target C57BL/6 stromal cells were cultured on the bottom well. After 10 days, target cells were harvested for RNA, and RT-qPCR for Collagen1a1, TGF-β, and CTGF genes were performed. FIG. 22D—Increasing number of senescent tdTOMp16+ cells (0, 1×10⁴, 2×10⁴, 4×10⁴, and 1×10⁵ cells) were plated on top well and the target C57BL/6 stromal cells were cultured on the bottom well. After 10 days, target cells were harvested for RNA and RT-qPCR for Collagen 1a1, Collagen 3, TGF-β, and CTGF genes was performed (n=3, *P=<0.05; **P=0.01, NS=non-significant). P values were calculated by t test.

FIG. 23A-FIG. 23C. In transwell culture, inhibition of tyrosine kinase Fgr by TL02-59 abrogates the induction of profibrotic genes in target cells. FIG. 23A—Irradiated tdTOMp16+ non-senescent (tdTOM−) and senescent (tdTOM+) cells were cultured on the top well of the transwell, and the target C57BL/6 stromal cells were cultured on the bottom well. Cells were treated with either vehicle or 10 nM Fgr inhibitor TL02-59 and after 10 days, target cells were harvested for RNA, and RT-qPCR was performed for TGF-β and Collagen 3 genes. FIG. 23B—Inhibition of Fgr phosphorylation by TL02-59 was confirmed by western blot from the vehicle and TL02-59-treated cell lysates of irradiated senescent (tdTOM+) cells (n=3, *P=<0.05; **P=0.01). FIG. 23C—Phosphorylation of Fgr was inhibited by TL02-59. NS nonsignificant), P values were calculated by t test.

FIG. 24A-FIG. 24C. shRNA inhibition of Fgr in senescent (tdTOM+) cells abrogates the induction of profibrotic genes. FIG. 24A—Validation of the efficacy of 3 shRNAs by RT-qPCR targeting the coding region of Fgr after 72 h of transient transfection in mouse bone marrow stromal cells. FIG. 24B—Validation of the efficacy of three shRNAs by western blotting after 72 h of by transient transfection in mouse bone marrow stromal cells. FIG. 24C—Stable control or Fgr shRNA knockdown tdTOMp16+ cells were irradiated and sorted non-senescent (tdTOM−) and senescent (tdTOM+) cells were cultured on top well of the transwell, and the target C57BL/6 stromal cells were cultured on the bottom well and after 10 days, target C57BL/6 cells were harvested for RNA and RT-qPCR was performed for TGF-β and Collagen 3 genes. (n=3, *P=<0.05; **P=0.01, NS non-significant). P values were calculated by t test.

FIG. 25A-FIG. 25D. Induction of Fgr and biomarkers of senescence in irradiated mouse lungs. FIG. 25A—951 Scheme showing the thoracic irradiation (20 Gy) and the days the lungs of the C57BL/6 mice 952 were harvested for total RNA. FIG. 25B—Expression of p21 and Fgr in the mouse lungs were evaluated 953 by RT-qPCR at different days. FIG. 25C—Expression of p16 and p19 in the mouse lungs was evaluated by RT-qPCR at different days. FIG. 25D—20 Gy irradiated mouse lungs tissue was immunostained using Fgr and Collagen antibodies (n=3-5 mice, *P=<0.05; **P=0.01), P values were calculated by ANOVA test).

FIG. 26A-FIG. 26D. Single-cell RNA-Seq analysis of mice with radiation-induced pulmonary fibrosis. FIG. 26A—Single-cell RNA-Seq was performed on single-cell suspensions generated from two control and two 150 days post-irradiated (20 Gy) irradiated mouse lungs with fibrosis. All samples were analyzed using canonical correlation analysis within the Seurat R package. Cells were clustered using a graph-based shared nearest-neighbor clustering approach and visualized using a U-map plot. Canonical cell markers were used to label clusters by cell identity as represented in the plot. Cell types were classified as indicated. FIG. 26B—Cellular populations represented in all four samples are shown in UMAP plot identified. Each sample was color-coded to show the differences in cell types represented by control and irradiated lungs. The distribution of senescent cells is shown for both control and irradiated lungs cell types in the UMAP plot. The expression of canonical 23 senescence-associated gene expressions were used to generate a senescence score for any individual cell and applied it to all cell types in the dataset. The lighter the blue, the more the expression of the genes. FIG. 26C—Distribution of Fgr expressing cells is shown for both control and irradiated lung cell types in the UMAP plot. FIG. 26D—Purple color indicates the expression of Fgr.

FIG. 27A-FIG. 27B. Irradiation-induced tdTOMp16+ senescent cells display common biomarkers of senescence. FIG. 27A—SA-beta-gal staining after 10 days after 0 and 5 Gy irradiation in tdTOMp16+ cells. FIG. 27B—Genetic markers (p16 and p21) after FACS sorting by RT-qPCR. (n=3, **, p=<0.0; ***, p=<0.001).

FIG. 28A-FIG. 28C. Irradiation-induced and sorted tdTOMp16+ senescent cells are non-dividing. FIG. 28A—Single cells in 96 well plates. FIG. 28B—tdTOM+, 5 Gy, tdTOM− and non-irradiated cells 10 days (% cell division). FIG. 28C—Single non-irradiated cell (left), single larger, circular irradiated tdTOM+ sorted cell (right) after 2 weeks. (n=600-1000; *, p=<0.5; **, p=<0.01).

FIG. 29A-FIG. 29B. Real time imaging of senescence. tdTOMp16+ cells irradiated (5 Gy), imaged hourly for 9 days FIG. 29A—tdTOM+ cells bigger, (arrowheads) surrounded by non-senescent cells. FIG. 29B—Senescent cells over 9 days. (IncuCyte® Live-Cell Analysis Systems from Essen BioScience, MI, USA, Lanigan™, et al.)

FIG. 30. RNA integrity of control cells and 5 Gy irradiated TOM+ cells and TOM− cells.

FIG. 31. Volcano plots showing Fgr expression when radiated senescent, radiated non-senescent, and nonirradiated cells are compared to each other in each pair. The plots show the log 2 (fold change) versus the −log 10 (adjusted pvalue) for all of genes detected in the RNA-seq analysis (i.e., for both DEGs and non-DEGs). The grey color represents genes with no significantly different expression while the red color indicated over expression or under expression. Place of Fgr is shown by an arrow in each of the three comparisons.

FIG. 32. Senescence biomarkers in tdTOMp16+ sorted cells from freshly explanted mouse tail skin fibroblasts. Irradiated (R) (5 Gy) and non-irradiated (NR) tdTOMp16+ cells on top and target fibroblasts on bottom. Target cells 10 days harvested for RNA and RT-qPCR for Ctgf, TGF-β, Collagen1a1, Collagen 3. (n=3, *, p=<0.05; **, p=0.01, NS=non-significant).

FIG. 33A-FIG. 33B. Confirmation of increased abundance of specific transcripts by RT-qPCR A) Relative expression of Fgr by RT-qPCR. B) SASP genes Ccl4, Ccl3, Ccl12 by RT-qPCR and RNA-seq. Error bars represent + s.d. Overall, the trend of gene expression by RT-qPCR mirrors that of the RNA-seq analysis. (n=3; **, p=<0.01; ***, p=<0.001).

FIG. 34. Clusters of populations with proportion of cells in each cluster on UMAP. Single-cell RNA-seq was performed on single-cell suspensions generated mice with control and pulmonary fibrosis. All cells were used to generate 21 different clusters and their assigned cell phenotypes.

FIG. 35. SASP genes in senescent cells in mouse lungs by scRNAseq. 23 major genes were shortlisted and analyzed for induction of senescence in different cell types identified by scRNAseq in control and RIPF mouse lungs. 23 genes were upregulated in every senescent cell.

FIG. 36. Fgr expression in RIPF lung cells at day 150 after 20 Gy thoracic irradiation compared to control lungs. scRNAseq analysis of Fgr expressing cell types shows upregulation of Fgr in neutrophils (p=1.6e-06), macrophages (p=0.07) and dendritic cells (two clusters, p=0.001 and p=0.0058).

FIG. 37. Crystalline silica induces senescence in the lung. Real time imaging of p16^{+/LUC} mice after intratracheal injection of crystalline silica (200 mg/kg). On days 6, 13, 20, and 27 after injection, the mice were injected intraperitoneally with D-Luciferin (150 mg/kg), the substrate for luciferase. Ten minutes later, the mice were imaged using IVIS Lumina XR.

FIG. 38. Silica induced epithelial senescence is associated with lung fibrosis. Control and silica-induced fibrotic mouse lungs were immuno-stained for p16 (green), alpha-smooth-muscle actin (α-SMA) (red) and Collagen1 (Col1, white). The upper two rows of photomicrographs show the airway of control mice and the lower two rows the airway of mice that received intratracheal silica (200 mg/kg). The inset in the silica-treated mice shows col1 deposition in the vicinity of α-SMA expressing cells that are behind the p16 positive green lung epithelium. Images are representative of 3 separate experiments.

FIG. 39A-FIG. 39B. Induction of senescence precedes fibrosis in both silica- and radiation-induced pulmonary fibrosis. FIG. 39A—RT-qPCR on silica injected lungs at different days (days 0, 7, 14, 21 and 28) showing a time-dependent increase of senescent markers p16 and p21. FIG. 39B) RT-qPCR on thoracic irradiated (20 Gy) mouse lungs performed at different days (days 0, 50, 75, 110 and 125) showing time-dependent increase of senescent markers p16 and p21 (n=2-5, t-test, *p≤⁰0.05; **p≤0.01; ***pS≤0.001).

FIG. 40A-FIG. 40D. Induction of tyrosine kinase Fgr in both silica- and radiation-induced pulmonary fibrosis. FIG. 40A—Silica injected mouse lungs were immunostained for p16 and tyrosine kinase Fgr. FIG. 40B—RT-qPCR on silica injected lungs (200 mg/kg) at different days (days 0, 7, 14, 21 and 28) showing a time-dependent increase of senescent marker Fgr. FIG. 40C—Thoracic irradiated (20 Gy) mouse lungs were immunostained for p16 and tyrosine kinase Fgr. FIG. 40D—RT-qPCR on thoracic irradiated (20 Gy) mouse lungs was performed at different days (days 0, 50, 75, 110, and 125) and showed a time-dependent increase of the senescent markers p16 and p21 (n=2−5·t-test, *p≤0.05; **p≤0.01; ***p≤0.001).

FIG. 41A-FIG. 41B. Senescent cell induction of migration of freshly explanted gfp+ mouse bone marrow and, specifically, gfp+ monocytes/macrophages in transwell cultures. FIG. 41A—gfp+ marrow. Cells on the top chamber of the transwell system were separated by a 3-micron filter from sorted tdTOMp16+ senescent cells on the bottom chamber. FIG. 41B—At 48 h, bone marrow cells migrating to the bottom chamber were counted and immunophenotyped for cell surface markers of monocytes/macrophages or other marrow cells. Cells in the bottom chamber of the transwell system were either red sorted senescent cells (green bar), or irradiated but non-senescent (non-red) cells from the same irradiated culture (blue). Results are presented as mean±SEM of three separate experiments (n=3; p≤0.05; p≤0.001). p-Values were calculated by t-test.

FIG. 42. Effect of silica on recruitment of monocytes into the lungs. Single suspensions of lung cells from control or silica-treated mice on day 3 or day 21 were first sorted for F4/80 positive (green) cells (panels in column A). The F4/80+ cells were analyzed for Ly6C and CCR2 and gated for 4 quadrants: (1) Ly6C− & CCR2+ cells, (2) Ly6C^{hi} & CCR2+ cells, (3) Ly6C− & CCR2+ cells, (4) Ly6C^{hi} & CCR2-cells (panels in column B). F4/80+, Ly6C^{hi} & CCR2+ cells and F4/80+, Ly6C− & CCR2− cells were further analyzed for CX3CR1 cells (Panels in column C).

FIG. 43A-FIG. 43D. Tyrosine kinase Fgr expression is induced in silica-induced senescent lung cells and in recruited bone marrow monocytes/macrophages. FIG. 43A—Relative percentage of red senescent cells sorted from silica-treated mouse lungs. FIG. 43B—Upregulation of Fgr was observed in senescent epithelial (Tom+, CD45−, CD326+) cells and senescent alveolar (Tom+, CD45+, F4/80+, CD1 lb−) macrophages. FIG. 43C—Relative percentage of bone marrow origin gfp+ macrophage/monocyte cells sorted from gfp chimeric bone marrow, and silica-treated tdTOMp16+ mouse lungs. FIG. 43D—Fgr expression was higher in gfp+ , CD45+, F4/80+, CD11 b− macrophages.

FIG. 44A-FIG. 44C. TL02-59 inhibits the induction by senescent cells of fibrosis biomarkers in target cells in transwell cultures. FIG. 44A—tdTOMp16+ cells were either irradiated (5 Gy, then sorted 10 days later) or non-sorted. The tdTOMp16+ cells were sorted for either tdTOM+ or tdTOM− cells and cultured at the top chamber of each transwell culture well. Target cells were C57BL/6 mouse marrow derived mesenchymal stem cells (MSC) that were cultured in the bottom chamber of each transwell culture separated by a 0.4-micron membrane. Bars show the non-sorted cells (blue bars), sorted TOM+ cells (red bars), or Fgr inhibitor, TL02-59, (10 nM) treated cells (green bars). Ten days later, the target bone marrow cells were harvested and assayed for biomarkers of fibrosis including: FIG. 44B—TGF-β; FIG. 44C—collagen III. (n=2−6, *p≤0.05; **p=0.01). p-Values were calculated by the t-test.

FIG. 45A-FIG. 45F. Inhibition of tyrosine kinase Fgr by TL02-59 prevents migration of bone marrow cells towards senescent cells. FIG. 45A—The tdTOMp16+ cells either irradiated or non-irradiated were sorted by FACS for either tdTOM+ or tdTOM− cells, and each population was placed in the bottom chamber of each culture, well separated by a 3-micron filter. Gfp+ bone marrow cells were placed on the top chamber of each well and migration assay was carried out (3-micron membrane). The Fgr inhibitor TL02-59 (10 nM) (Shen K et al. Sci Signal 11(553): eaat5916, 2018) or RS 504393 (10 μM), a Ccl2 inhibitor, which is a CCR2 chemokine receptor antagonist, was added to cultures 24 h later. The migration of gfp+ bone marrow cells from the top chamber through the 3-micron filter to the bottom chamber was imaged and total gfp+ cells were counted. FIG. 45B—Image of gfp+ migrating cells through the filter at 24 h for each condition (×100). At 48 h, emigrated gfp+ cells in the bottom well were counted by FACS. After 48 h, migrating total. (FIG. 45C) gfp+ cells, and (FIG. 45D) gfp+ CD45+ cells were counted by FACS. FIG. 45E—Senescent tdTOM+ lung cells sorted from irradiated mouse lungs were plated at the bottom wells and Fgr kinase inhibitor (TL02-59, 10 nM) or Ccl2-inhibitor RS 504393 (Ccl2i, 10 μM) was added for 12 h before adding GFP positive mouse bone marrow cells on the top wells. Cell migration from the top wells to the bottom wells was evaluated by imaging and by (FIG. 45F) FACS analysis. (n=3, ****p=0.0001). p-Values were calculated by the t-test.

FIG. 46A and FIG. 46B. Representative images of control (top row panels), irradiated (18 Gy) (middle row panels), and irradiated plus TL02-59 treated (bottom row panels) mouse lungs stained using H&E (FIG. 46A) or Trichrome (FIG. 46B).

FIG. 47. Fgr inhibitor TL02-59 treatment reduces profibrotic and biomarkers of senescence in a mouse model of radiation-induced pulmonary fibrosis. Relative mRNA expression of profibrotic genes (Tgf-β, Ctgf, Collagen 1a1, Collagen 3 and Collagen 4), senescence biomarkers (p16 and p21) and tyrosine kinase Fgr was evaluated by qPCR from one lobe of mouse lungs in control, irradiated and treated with Fgr inhibitor TL02-59 post irradiation. Briefly, Mice were irradiated (18 Gy), and TL02-59 treated (10 mg/kg), 3/week at day 75 for 4 weeks. (n=4-13). Statistics was performed, and individual p values were calculated by unpaired t-test.

FIG. 48. Expression of Fgr and p16 from mouse lungs in control, irradiated, and treated with Fgr inhibitor TL02-59 post irradiation. Images show induction of Fgr and senescence marker Fgr upon irradiation and TL02-59 treatment led to a reduction of both the proteins. Insets on the right from each group were magnified for clarity.

FIG. 49. Induction of Fgr, p16, and α-SMA in cells within human RIPF. Surgically resected lung of a radiotherapy patient at 24-months after 60 Gy treatment for lung cancer, and age matched control lungs were stained for Fgr, p16, α-SMA and nuclear stain DAPI (20×). Images are representative of 3 separate patient samples.

FIG. 50. Fgr inhibitor TL02-59 reduces secretion of senescence associated secretory chemokines. tdTOMp16 bone marrow stromal cells were irradiated (5 Gy) and after 10 days tdTOMp16+ red senescent cells and non-red non-senescent were FACS sorted and cultured with TL02-59 (10 nM) for 72 hours. Using chemokine array relative amounts of chemokines secreted in the media was measured. (n=2, *, p<0.05, p values were calculated using unpaired t-test).

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a disease” includes mixtures of two or more such diseases, reference to “the composition” includes two or more such compositions, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., Fgr expression). It is understood that this is typically in relation to some standard or expected value, in other words it is relative, but that it is not always necessary for the standard or relative value to be referred to. For example, “reduces expression” means decreasing the amount of one or more specific biomarkers relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as radiation-induced fibrosis), diminishment of extent of radiation-induced fibrosis, stabilized (i.e., not worsening) state of radiation-induced fibrosis, preventing or delaying radiation-induced fibrosis, delaying occurrence or recurrence of fibrosis, delay or slowing of fibrosis progression, amelioration of the fibrosis state (including general symptoms), and remission (whether partial or total).

As used herein, “inhibit” or “inhibition” refers to the activity of a gene expression product or level of RNAs or equivalent RNAs encoding one or more gene products is reduced below that observed in the absence of an inhibitor as described herein. The term “expression” with respect to a gene refers to transcription of the gene and, as appropriate, translation of the resulting mRNA transcript to a protein. Thus, as will be clear from the context, expression of a protein coding sequence results from transcription and translation of the gene.

The term “silencing” as used herein refers to suppression of expression of the (target) gene. It does not necessarily imply reduction of transcription. The degree of gene silencing can be complete so as to abolish production of the encoded gene product (yielding a null phenotype), or the gene expression is partially silenced, with some degree of expression remaining (yielding an intermediate phenotype).

The term “patient” preferably refers to a human in need of treatment for any purpose, and more preferably a human in need of such a treatment for fibrosis. However, the term “patient” can also refer to non-human animals, preferably mammals such as dogs, cats, horses, cows, pigs, sheep and non-human primates, among others, that are in need of treatment.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases. e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to fibrosis, an effective amount comprises an amount sufficient to (i) reduce fibrotic processes; (ii) inhibit, retard, slow to some extent and preferably stop fibrotic processes; (iii) prevent or delay occurrence and/or recurrence of fibrotic processes; and/or (iv) relieve to some extent one or more of the symptoms associated with fibrotic processes. An effective amount can be administered in one or more doses.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

Reference will now be made in detail to specific aspects of the disclosed materials, compounds, compositions, articles, and methods, examples of which are illustrated in the accompanying Examples.

### Compounds, Compositions, and Methods

Compounds, compositions, and methods for treating or preventing fibrosis in a subject in need thereof are disclosed. Fibrosis processes are driven by a cascade of injury, inflammation, fibroblast proliferation and migration, and matrix deposition and remodeling. Studies have shown that cellular senescence plays a role in the pathogenesis of fibrosis disorders, such as idiopathic pulmonary fibrosis, scleroderma, diabetic nephropathy, glomerulosclerosis, cirrhosis, and diabetic retinopathy. The term “senescence” or “cellular senescence” refers to the progression from an actively dividing cell to a metabolically active, non-dividing cell. The term also refer to the state into which cells enter after multiple rounds of division and, as a result of cellular pathways, future cell division is prevented from occurring even though the cell remains metabolically active.

Senescent cells have also been implicated in radiation-induced pulmonary fibrosis, as well as, in idiopathic pulmonary fibrosis, and senolytic drugs have been shown to improve lung function in experimental fibrosis models. Still, the molecular mechanism by which senescent cells regulate fibrosis was unknown, until the surprising discovery disclosed herein. As demonstrated herein, Fgr, a member of Src family kinases, is upregulated in fibrosis senescent cells, than that of any other transcript including those associated with chemotaxis or phagocytosis. In fact, it is shown herein that Fgr is increased in senescent cells in situ in irradiated mouse lung before the appearance of fibrosis.

Fgr biomarker in senescent cells can be targeted to treat or prevent aberrant fibroblast proliferation, extracellular matrix deposition, and particularly fibrosis conditions. A senescent cell as described herein may be senescent due to replicative cellular senescence, premature cellular senescence or therapy-induced senescence. A senescent cell that is prematurely cellular senescent may be induced by, but not limited to, ultraviolet light, reactive oxygen species, chemotherapeutics, environmental toxin, cigarette smoking, ionizing radiation, distortion of chromatin structure, excessive mitogenic signaling, and oncogenic mutations. In a specific embodiment, premature cellular senescence may be induced by ionizing radiation (IR). Radiation induced senescence (RIS) differs from oncogene or replication induced senescence. A senescent cell may generally be a eukaryotic cell. Non-limiting examples of senescent cells may include, but are not limited to, mammary epithelial cells, keratinocytes, cardiac myocytes, chondrocytes, endothelial cells (large vessels), endothelial cells (microvascular), epithelial cells, fibroblasts, follicle dermal papilla cells, hepatocytes, melanocytes, osteoblasts, preadipocytes, primary cells of the immune system, skeletal muscle cells, smooth muscle cells, adipocytes, neurons, glial cells, contractile cells, exocrine secretory epithelial cells, extracellular matrix cells, hormone secreting cells, keratinizing epithelial cells, islet cells, lens cells, mesenchymal stem cells, pancreatic acinar cells. Paneth cells of the small intestine, primary cells of hemopoietic linage, primary cells of the nervous system, sense organ and peripheral neuron supporting cells, wet stratified barrier epithelial cells and stem cells. In a specific embodiment, a senescent cell of the invention is a fibroblast.

In some aspects, the disclosed subject matter relates to compounds, compositions, and methods for treating and preventing a disease or condition characterized by aberrant fibroblast proliferation and extracellular matrix deposition in a tissue of a subject in need thereof. The disease or condition characterized by aberrant fibroblast proliferation and extracellular matrix deposition can result from idiopathic pulmonary fibrosis (IPF), pneumonia, acute respiratory distress syndrome (ARDS), asbestosis, bleomycin exposure, silicosis, anthracosis, post bacterial infectious fibrosis, viral (including Covid-19) liver fibrosis, post heat burn fibrosis, post ultraviolet light fibrosis, post trauma fibrosis, myocardial infarction, injury related tissue scarring, scarring form surgery, radiation exposure, allergic reaction, inhalation of environmental particulates, smoking, infection, mechanical damage, transplantation, autoimmune disorder, genetic disorder, a disease condition (such as scleroderma lung disease, rheumatoid arthritis, sarcoidosis, tuberculosis, Hermansky Pudlak Syndrome, bagassosis, systemic lupus erythematosis, eosinophilic granuloma, Wegener's granulomatosis, lymphangioleiomyomatosis, or cystic fibrosis), nitrofurantoin exposure, amiodarone exposure, cyclophosphamide exposure, methotrexate exposure, or a combination thereof.

In some examples of the disclosed methods, the disease or condition is fibrosis. The particular fibrosis disease or condition can be idiopathic pulmonary fibrosis (IPF), asbestosis, silicosis, anthracosis, post bacterial infectious fibrosis, viral (including Covid-19) liver fibrosis, post heat burn fibrosis, post ultraviolet light fibrosis, post trauma fibrosis, scleroderma, scarring, liver fibrosis, kidney fibrosis, gut fibrosis, radiation-induced fibrosis, bleomycin-induced fibrosis, asbestos-induced fibrosis, biliary duct injury-induced fibrosis, head and neck fibrosis, burn induced fibrosis, surgical fibrosis, spinal cord fibrosis, or lung fibrosis. In specific examples, the fibrosis disease or condition is lung fibrosis, such as radiation-induced fibrosis.

Radiation-induced fibrosis is an after-effect of exposure of a human or animal body to radiation. The radiation can be an ionizing radiation resulting from radiation therapy for neoplastic disease, or may result from exposure to ionizing radiation from other sources such as radionuclide contamination, nuclear weapons, mining activities, accidental exposures from nuclear power generation, industrial hazards, or other exposures to ionizing radiation. Ionizing radiation is highly-energetic particles or waves that can detach (ionize) at least one electron from an atom or molecule. Examples of ionizing radiation are energetic beta particles, neutrons, and alpha particles.

Electrons, x rays, gamma rays or atomic ions may be used in radiation therapy to treat neoplastic disease, including malignant tumors (cancer). Medical procedures, such as diagnostic X-rays, nuclear medicine, and radiation therapy are by far the most significant source of human-made radiation exposure to the general public. Some of the major radionuclides used are I¹³¹ Tc⁹⁹, Co⁶⁰, Ir¹⁹², and Cs¹³⁷. Humans and animals are exposed to radiation from consumer products, such as tobacco (Po²¹⁰), building materials, combustible fuels (gas, coal, etc.), ophthalmic glass, televisions, luminous watches and dials (H³), airport X-ray systems, smoke detectors (Americium (Am)), road construction materials, electron tubes, fluorescent lamp starters, and lantern mantles (thorium (Th)). Occupationally exposed individuals are exposed according to the sources with which they work. Some of the radionuclides of concern include Co⁶⁰, Cs¹³⁷, Am²⁴¹, and I¹³. Examples of industries where occupational exposure is a concern include airline crew, industrial radiography, nuclear medicine and medical radiology departments (including nuclear oncology), nuclear power plants and research laboratories.

Radiation induced lung injury is the main dose-limiting factor when irradiating the lung, for example, for treatment of tumors or neoplastic disease such as lung cancer, Hodgkin's lymphoma or non-Hodgkin's lymphoma. As such, organ or tissue tolerance limits the therapeutic options for treatment of cancer or neoplastic disease. Although the advent of more sophisticated radiation therapy techniques, such as three dimensional conformal radiation therapy or intensity-modulated radiation therapy, can permit dose escalation by limiting the normal tissue complication probability, radiation-induced fibrosis has not been eliminated, and therapy for, or prevention of, radiation-induced fibrosis presents a continuing problem.

Radiation-induced fibrosis in the lung may manifest as two distinct, though potentially connected, abnormalities. One manifestation is radiation pneumonitis, which is an early inflammatory reaction involving alveolar cell depletion and inflammatory cell accumulation in the interstitial space that occurs within 12 weeks after lung radiation therapy. The second manifestation is a late phase of radiation-induced fibrosis, considered until recently as irreversible, that consists mainly of fibroblast proliferation, collagen accumulation, and destruction of the normal lung architecture. Although studies have attempted to elucidate the mechanisms leading to radiation-induced fibrosis, the pathogenesis of radiation-induced fibrosis lung injury, and other radiation-induced fibrosis injury, at the cellular and molecular level to date has been incompletely described.

As described herein, cellular senescence has been shown to be a regulator of fibroblast proliferation, cell adhesion, and the stimulation of extracellular matrix production. Accordingly, in some aspects of the present disclosure, methods for treating or preventing a disease or condition characterized by aberrant fibroblast proliferation and extracellular matrix deposition in a tissue of a subject in need thereof, wherein the disease or condition is mediated by Fgr, are disclosed. In some examples, the disease or condition is radiation-induced fibrosis.

In further aspects, methods for treating or preventing radiation-induced fibrosis in a subject in need thereof are also disclosed. The radiation-induced fibrosis can be an ionizing radiation-induced fibrosis, such as radiation therapy for cancer treatment. In other further aspects, methods for preventing fibrosis or a disease or condition mediated by Fgr in a subject exposed to ionizing radiation are disclosed.

The methods for treating and preventing a disease or condition characterized by aberrant fibroblast proliferation and extracellular matrix deposition in a tissue, for preventing fibrosis or a disease or condition mediated by Fgr in a subject exposed to ionizing radiation, or for treating or preventing radiation-induced fibrosis can include administering to the subject an effective amount of a composition that inhibits Fgr (a non-receptor tyrosine kinase).

Fgr belongs to the Src-family kinase, which are the Src proto-oncogene, non-receptor tyrosine kinases. The composition that inhibits Fgr as disclosed herein can include a pharmacological compound that inhibits non-receptor tyrosine kinase or a tyrosine kinase. For example, the composition can include a small pharmacological molecule such as a N-phenylbenzamide tyrosine kinase inhibitor. A specific example of a N-phenylbenzamide tyrosine kinase inhibitor that inhibits Fgr is TL02-59. In other embodiments, the composition that inhibits Fgr can comprise a nucleic acid molecule such as shRNA that interferes with Fgr expression; CRISPR for editing or knockout of Fgr; siRNA, antisense RNA, or nucleic acids that interfere with or prevent the transcription or translation of Fgr genes; antisense molecules comprising Fgr sequences; active sites that bind at least a portion of Fgr; interfering peptides; interfering nucleic acids; or a combination thereof. For example, the composition that inhibits Fgr can include shRNA that interferes with Fgr expression, specifically shRNA mediated knockdown of Fgr inhibits induction of biomarkers of radiation fibrosis in target cells. The term “shRNA” as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. As described herein, expression or activity of the Fgr can be inhibited by the use of interfering nucleic acid, such as shRNA.

Other inhibitors of Src non-receptor tyrosine kinase, and specifically Fgr are described in US Publication No. 2017/0275593A1, which is incorporated herein by reference in its entirety. For example, inhibitors of Src kinases can include dasatinib, SU6656, CGP77675, Bosutinib, WH-4-023, and AZD05530. Other suitable compounds possessing inhibitory activity against the Src family of non-receptor tyrosine kinases include the quinazoline derivatives disclosed in International Patent Applications WO 2001/94341, WO 2002/16352, WO 2002/30924, WO 2002/30926, WO 2002/34744, WO 2002/085895, WO 2002/092577, WO 2002/092578, WO 2002/092579, the quinoline derivatives described in WO 2003/008409, WO 2003/047584, WO 2003/048159, and the quinazoline derivatives described in European Patent Applications 02292736.2 and 03290900.4. The 4-anilino-3-cyanoquinoline Src inhibitor known as SKI 606 is described in Cancer Research, 2003, 63, 375. Other compounds which possess Src kinase inhibitory properties are described in, for example, International Patent Applications WO 1996/10028, WO 1997/07131, WO 1997/08193, WO 1997/16452, WO 1997/28161, WO 1997/32879, and WO 1997/49706. Other compounds which possess Src kinase inhibitory properties are described in, for example, J Bone Mineral Research, 1999, 14 (Suppl. 1), S487, Molecular Cell, 1999, 3, 639-647, Journal Medicinal Chemistry. 1997, 40, 2296-2303. Journal Medicinal Chemistry. 1998, 41, 3276-3292 and Bioorganic & Medicinal Chemistry Letters, 2002, 12, 1361 and 3153. Other compounds which may possess Src kinase inhibitory properties are described in, for example, International Patent Applications WO 02/079192, WO 03/000188, WO 03/000266, WO 03/000705, WO 02/083668, WO 02/092573. WO 03/004492, WO 00/49018, WO 03/013541, WO 01/00207, WO 01/00213 and WO 01/00214. Particular Src inhibitors include those provided in International Patent Application WO 01/94341. Further particular Src inhibitors include the following compounds from International Patent Application WO 2002/16352, WO 2002/30924, WO 2002/30926, and WO 2002/34744.

In some methods, a small molecule Fgr inhibitor (such as TL02-59) may be used in addition to other inhibitory compositions including, but not limited to, shRNA that interferes with Fgr expression; CRISPR for editing or knockout of Fgr; siRNA, antisense RNA, or nucleic acids that interfere with or prevent the transcription or translation of Fgr genes; antisense molecules comprising Fgr sequences; active sites that bind at least a portion of Fgr; interfering peptides; interfering nucleic acids; or a combination thereof. For example, the composition that inhibits Fgr can include shRNA that interferes with Fgr expression, specifically shRNA mediated knockdown of Fgr inhibits induction of biomarkers of radiation fibrosis in target cell.

One or more additional anti-fibrotic agent can be administered to the subject. The additional anti-fibrotic agent can be selected from the group consisting of calcium channel blockers, cytotoxic agents, cytokines, chemokines, integrins, growth factors, hormones, lysophosphatidic acid (LPA) receptor 1 antagonists, agents that modulate the TGF-β pathway, endothelin receptor antagonists, agents that reduce connective tissue growth factor (CTGF) activity, matrix metalloproteinase (MMP) inhibitors, agents that reduce the activity of platelet-derived growth factor (PDGF), agents that interfere with integrin function, agents that interfere with the pro-fibrotic activities of cytokines, agents that reduce oxidative stress, PDE4 inhibitors, PDE5 inhibitors, mTOR inhibitors, modifiers of the arachidonic acid pathway, peroxisome proliferator-activated receptor (PPAR)-γ agonists, kinase inhibitors, inhibitors of VEGF signaling pathway, matrix metalloproteinases, tissue inhibitors of metalloproteinases (TIMPs), HGF agonists, angiotensin-converting enzyme (ACE) inhibitors, angiotensin receptor antagonists, inhibitors of advanced glycation endproducts (AGEs) or their receptors (RAGEs), Rho kinase inhibitors, PKC inhibitors, ADAM-10 inhibitor, famesoid X receptor agonists, caspase inhibitors, anti-oxidants, inhibitors of collagen expression, LMW heparin or heparin analogs, copper chelators. TNF-α blocking agents, HMG-CoA reductase inhibitors, and Thy-I (CD90) inhibitors. The subject in need thereof can be human.

In an exemplary embodiment, the compounds, compositions, and methods disclosed herein are for treating or preventing radiation-induced pulmonary fibrosis (RIPF). The data herein using RNA-seq analysis with a pure population of sorted RIS cells expressing the tdTOMp16+ reporter gene, show that there is significant upregulation of a unique p15 tyrosine kinase, Fgr, in RIS cells. The degree of upregulation (seventy two-fold) is greater than that of any other gene including those involved in chemotaxis or phagocytosis. The data with a transwell co-culture system show that RIS cells induce biomarkers of fibrosis including collagen 1a, collagen 3, and TGF-β in separated target cells. The small molecule inhibitor of Fgr, TL02-59, was shown to abrogate the induction of biomarkers of fibrosis by RIS cells. The data also show that Fgr is detected in senescent cells in irradiated mouse lung in situ significantly before the appearance of fibrosis. There is currently no report of Fgr involvement in RIPF.

The methods for treating or preventing radiation-induced fibrosis in a subject can include administering the composition that inhibits Fgr before the subject undergoes ionizing radiation therapy, during the period the subject is undergoing ionizing radiation therapy, or after the subject undergoes ionizing radiation therapy or combinations of these. For example, prior to radiation therapy, the subject can be administered an effective amount of one or more Fgr inhibitors, such as in an oral dosage form. During radiation therapy, the subject can be administered an effective amount of one or more Fgr inhibitors in an oral dosage form and optionally, also in a dosage form that supplies an amount of inhibitor compound to an effected area, such as by inhalation for thoracic or lung radiation, and after radiation therapy, for a continuous period of time, the subject can be administered an oral dosage form of one or more Fgr inhibitors, optionally an inhalation dosage form, and optionally a topical dosage form comprising one or more Fgr inhibitors.

Administering the composition as described herein can be carried out orally, parenterally, periadventitially, subcutaneously, intravenously, intramuscularly, intraperitoneally, by inhalation, by intranasal instillation, by implantation, by intracavitary or intravesical instillation, intraocularly, intraarterially, intralesionally, transdermally, intradermally or by application to mucous membranes.

Methods for identifying a pharmacological compound for treating or preventing fibrosis in a subject in need thereof are also disclosed. The methods can include obtaining a population of p16 or β-galactosidase (SA-β-gal) expressing senescent cells from the subject. In some embodiments, the population of radiation induced p16 or β-galactosidase (SA-β-gal) expressing senescent cells can be pure or substantially pure, such as at least 80% purity, at least 85% purity, at least 90% purity, or at least 95% purity. In some examples, obtaining a population of radiation induced p16 or β-galactosidase (SA-β-gal) expressing senescent cells is by fluorescence activated cell sorting (FACS). The methods for identifying a pharmacological compound for treating or preventing fibrosis can further include identifying Fgr positive senescent cells. In some embodiments, Fgr positive senescent cells can be isolated. The methods can also include contacting the Fgr positive senescent cells with the pharmacological compound being tested and determining whether the pharmacological compound inhibits Fgr in the Fgr positive senescent cells.

The diagnostic methods can also be used for identifying a pharmacological compound for treating or preventing radiation induced fibrosis in a subject in need thereof. For example, the methods can include obtaining a population of radiation induced p16 or β-galactosidase (SA-β-gal) expressing senescent cells from the subject; identifying Fgr positive senescent cells, contacting the Fgr positive senescent cells with the pharmacological compound, and determining whether the pharmacological compound inhibits Fgr in the Fgr positive senescent cells.

Compositions, such as N-phenylbenzamide tyrosine kinase inhibitor, specifically TL02-59 or derivatives thereof, administered intranasally or by inhalation can be delivered in the form of a dry powder inhaler or an aerosol spray presentation from a pressurized container, pump, spray, nebulizer, inhaler, meter-dosed inhaler, dry powder inhaler, vibrating mesh nebulizer, jet nebulizer, or ultrasonic wave nebulizer, with the use of a suitable propellant, carbon dioxide or other suitable gas. The propellant can be selected from compressed air, ethanol, nitrogen, carbon dioxide, nitrous oxide, hydrofluoroalkanes (HFA), 1,1,1,2,-tetrafluoroethane, 1,1,1,2.3,3,3-heptafluoropropane or combinations thereof. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are provided so that each metered dose contains a suitable quantity of an inhibitor compound or composition, such as TL02-59 or derivatives thereof, for delivery to the subject. It will be appreciated that the overall daily dose with an aerosol will vary from patient to patient, and may be administered in a single dose or, more usually, in divided doses throughout the day. Nanoparticulated compounds or compositions, such as statins or statin derivatives, may be prepared using techniques known in the art.

Pharmaceutical compositions for the treatment or prevention of fibrosis comprising a pharmaceutically acceptable excipient, a therapeutically effective amount of a N-phenylbenzamide tyrosine kinase inhibitor, such as TL02-59, and a propellant are disclosed. A pressurized container comprising the pharmaceutical compositions described herein are disclosed.

### Administration

The term “administration” and variants thereof (e.g., “administering” a compound) in reference to a compound disclosed herein means introducing the compound or a prodrug of the compound into the system of the animal in need of treatment. When a compound or prodrug thereof is provided in combination with one or more other active agents, “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, inhaled, nasal, rectal, topical, and parenteral routes of administration. As used herein, the term parenteral includes subcutaneous, intradermal, intravenous, intramuscular, intraperitoneal, intraventricular and intrasternal administration, such as by injection. Administration of the disclosed compounds or compositions can be a single administration, or at continuous or distinct intervals as can be readily determined by a person skilled in the art.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient, and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/01201(0) disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer; poly[bis(p-carboxyphenoxy) propane:sebacic acid] in a 20:80 molar ratio (as used in GLIADEL); chondroitin; chitin; and chitosan.

For the treatment of fibrosis, the compounds disclosed herein can be administered to a patient in need of treatment in combination with other active coagents. These other substances or treatments can be given at the same as or at different times from the compounds disclosed herein.

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of injury (such as a site of brain injury), optionally in combination with a pharmaceutically acceptable carrier such as an inert diluent. Compounds and compositions disclosed herein can be systemically administered, such as intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally, intraventricularly, by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For administration by inhalation, the composition can be conveniently delivered from an insufflator, nebulizer pressurized packs or other convenient means of delivering an aerosol spray. Pressurized packs can comprise a suitable propellant such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Alternatively, for administration by inhalation or insufflation, the compounds can take the form of a dry powder composition, for example a powder mix of the compound and a suitable powder base such as lactose or starch. The powder composition can be presented in unit dosage form, in for example, capsules, cartridges, gelatin or blister packs from which the powder can be administered with the aid of an inhalator or insufflator.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

### Kits

Kits for practicing the methods of the invention are further provided. By “kit” is intended any manufacture (e.g., a package or a container) comprising at least one reagent, e.g., anyone of the compounds described herein. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight. The precise amount of composition administered to an individual will be the responsibility of the attendant physician. The specific dose level for any particular individual will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration can vary depending on the condition and its severity. The dosage can be increased or decreased over time, as required by an individual. An individual initially can be given a low dose, which is then increased to an efficacious dosage tolerable to the individual.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Mitigation of Radiation Pulmonary Fibrosis by Inhibition of Senescence Protein Fgr

Abstract: This example below provides a new mitigator of one component of the delayed effects of acute radiation exposure (DEARE), namely, radiation induced pulmonary fibrosis (RIPF). Recent data suggest that radiation induced cellular senescence plays an important role in RIPF, and that clearance of senescent cells (SCs) by senolytic drugs could be an effective therapeutic strategy. However, this approach has been unclear because the underlying molecular mechanism by which radiation induced senescence (RIS) triggers fibrosis is unknown. RIS cells exit the cell cycle, secrete components of a senescence associated secretory phenotype (SASP), and express p16 protein and senescence associated β-galactosidase (SA-β-gal). RIS differs from oncogene or replication induced senescence. Gene expression patterns unique to RIS may be responsible for triggering lung fibrosis. The data herein using RNA-seq analysis with a pure population of sorted RIS senescent cells expressing the tdTOMp16+ reporter gene, show that there is significant upregulation of a unique p15 tyrosine kinase, Fgr, in RIS cells. The degree of upregulation is greater than that of any other gene including those involved in chemotaxis or phagocytosis. The data herein with a transwell co-culture system show that RIS cells induce biomarkers of fibrosis including collagen 1a, collagen 3, and TGF-β in separated target cells. Furthermore, a small molecule inhibitor of Fgr, TL02-59, abrogates the induction of biomarkers of fibrosis by RIS cells. The data also show that Fgr is detected in senescent cells in irradiated mouse lung in situ significantly before the appearance of fibrosis. There is currently no report of Fgr involvement in RIPF. Other tyrosine kinases (PDGF receptor, VEGF receptor, EGF receptor, and JAK kinases) as well as non-receptor tyrosine kinases (c-Abl, c-Kit, and Src kinases) are not increased in RIS cells. Fgr, is a member of Src family kinases the ablation of which should reduce RIPF.

The specific methods below provides methods for establishing that genetic or pharmacologic inhibition of Fgr reduces RIPF.

Specific Method 1 will demonstrate that shRNA mediated knockdown of Fgr in RIS cells inhibits their capacity to induce biomarkers of radiation fibrosis in target cells. Specific Method 2 will establish that sorted RIS cells from the lungs of 20 Gy thoracic irradiated tdTOMp16+ mice express Fgr prior to detection of RIPF. Specific Method 3 will establish that administration of the Fgr inhibitor TL02-59 prevents RIPF in C57BL/6 mice. These studies will identify and lead to development of a new small molecule mitigator of radiation pulmonary fibrosis.

Introduction: Radiation induced senescence has been linked to, but not proven to be the cause of lung fibrosis. Described herein are purified radiation induced senescent cells by use of a red-fluorochrome reporter in cells from tdTOMp16+ mice, which were shown that they are non-dividing and express high levels of a tyrosine kinase protein, Fgr, which induces biomarkers of fibrosis in target cells. The specific methods below provides methods for demonstrating that genetic or pharmacologic inhibition of Fgr reduces radiation pulmonary fibrosis.

Survivors of the acute radiation syndromes (ARS) present with delayed effects of acute radiation exposure (DEARE), which include radiation-induced pulmonary fibrosis (RIPF). RIPF is also a side effect of clinical radiotherapy for lung cancer patients. Herein, a tyrosine kinase, Fgr, is identified by RNA-seq analysis of sorted tdTOMp16+ mouse RIS cells, which is significantly upregulated to a level greater than that of any other transcript including those associated with chemotaxis or phagocytosis. RIS cells induce biomarkers of fibrosis in separated target cells in transwell cultures in vitro. Fgr is increased in RIS cells in situ in irradiated mouse lung before the appearance of fibrosis. As shown herein, it was established that genetic inhibition of Fgr by shRNA knockdown and pharmacologic inhibition of Fgr by a specific small molecule TL02-59 prevent RIPF. To achieve this goal, a primary stromal cell line from tdTOMp16+ mice was established and sorted into a pure population of radiation induced p16 expressing red senescent cells by fluorescence activated cell sorting (FACS). RNA-seq on this pure population of RIS cells was performed and it was shown that gene expression profiles of RIS cells are distinct from both irradiated non-senescent and non-irradiated cells. It has also been shown that RIS cells induce biomarkers of fibrosis in separated target cells in transwell cultures. Furthermore, shRNA knockdown of Fgr has been achieved and the Fgr inhibitor TL02-59 has been shown to abrogate the induction of fibrotic biomarkers in target cells. C57BL/6^{tdTom}p16+ mice develop pulmonary fibrosis at 180 days after 20 Gy thoracic irradiation, and it was evidenced that Fgr positive senescent cells appear in significant numbers in the lungs significantly earlier. In further studies, Fgr positive senescent cells can be sorted and isolated from irradiated lungs, their phenotype (epithelial, endothelial, immunocyte) determined, and compared by RNA-seq to RIS cells sorted from overlying thoracic skin and to irradiated cell lines in vitro. This can establish that inhibition of Fgr by administration of TL02-59 to mice prevents radiation pulmonary fibrosis.

Specific Method 1: Elucidate the mechanism by which downregulation of Fgr in RIS cells reduces their capacity to induce biomarkers of radiation fibrosis. Specific Method 2: Determine the phenotype(s) of Fgr positive RIS cells, which appear in the irradiated lung prior to and during the onset of fibrosis. Specific Method 3: Determine the timing of administration of the Fgr inhibitor TL02-59 to ameliorate RIPF. These specific experiments can establish the role of senescence in RIPF and identify a new pulmonary radiation countermeasure.

General Methods and Results: There is currently no direct proof that RIS cells induce radiation pulmonary fibrosis. Using a tdTOMp16+ mouse bone marrow stromal cell line, a pure population of red tdTOMp16+ senescent cells was sorted by FACS at 10 days after 5 Gy irradiation and RNA-seq, was performed. It was confirmed that there is enrichment of SASP components and a 72-fold upregulated level of a transcript for a profibrotic tyrosine kinase. Fgr, was identified. A non-contact transwell co-culture system is to be used to establish the antifibrotic effects of both shRNA knockdown of Fgr and adding an inhibitor of Fgr. Fgr positive senescent cells were identified in the lungs of 20 Gy thoracic irradiated mice in vivo prior to the appearance of fibrosis. Both genetic and pharmacologic inhibition of Fgr can be used to abrogate the biologic effects of RIS in vitro and RIPF in vivo. shRNA knockdown of Fgr in irradiated senescent cells (plated on top in a transwell culture system) over a monolayer of C57BL/6 mouse stromal target cells (bottom). Irradiation-induced Fgr in RIS cells was downregulated by shRNA to abrogate their capacity to induce biomarkers of fibrosis. Using the small molecule library of Medchemexpress and published literature. TL02-59, was identified which selectively inhibits Fgr. The Fgr stimulated and secreted factors (by proteomics analysis of conditioned medium from RIS cells) that induce biomarkers of fibrosis in transwells in vitro can be identified and those which are absent from the same RIS cells after Fgr shRNA knockdown can be revealed. To confirm the results with shRNA knockdown of Fgr, TL02-59 to transwell cultures can be added and the conditioned medium analyzed for the absence of profibrotic signals. The phenotype(s) of senescent cells in 20 Gy thoracic irradiated tdTOMp16+ mouse lungs can be determined, which show upregulated Fgr. Red RIS cells can be explanted, sorted, and purified from the lungs of tdTOMp16+ mice and RNA-seq performed at serial times after 20 Gy thoracic irradiation to confirm that Fgr is produced by RIS cells before fibrosis. It can be established that RIS lung cells produce profibrotic factors in transwell experiments and these factors identified by proteomics analysis of conditioned medium. It can be established that TL02-59 inhibits fibrosis in vivo by reducing levels of profibrotic products that are produced by Fgr positive RIS cells. TL02-59 can be administered to tdTOMp16+ mice beginning at times after 20 Gy thoracic irradiation when senescent cells are first detected and the time course of effective amelioration of fibrosis established. Then CRISPR knockout of Fgr to prevent RIPF in mice can be performed.

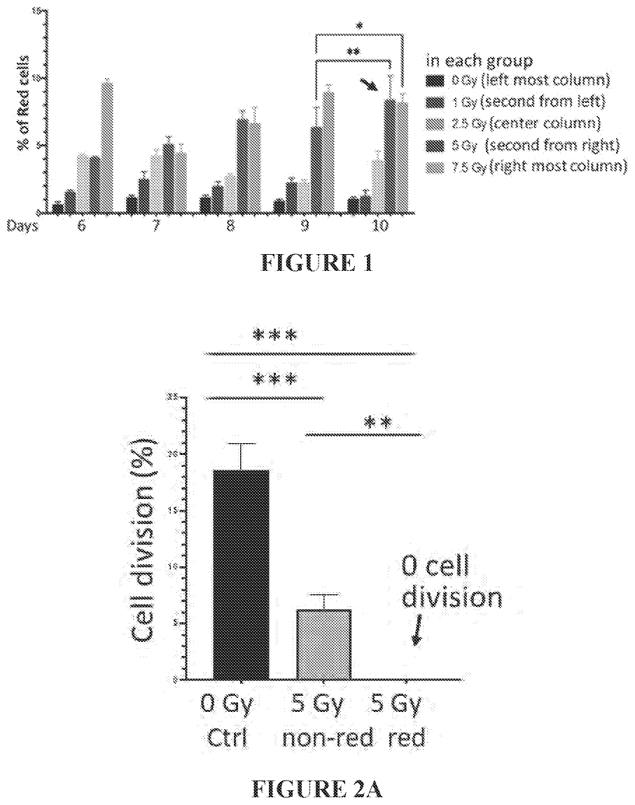

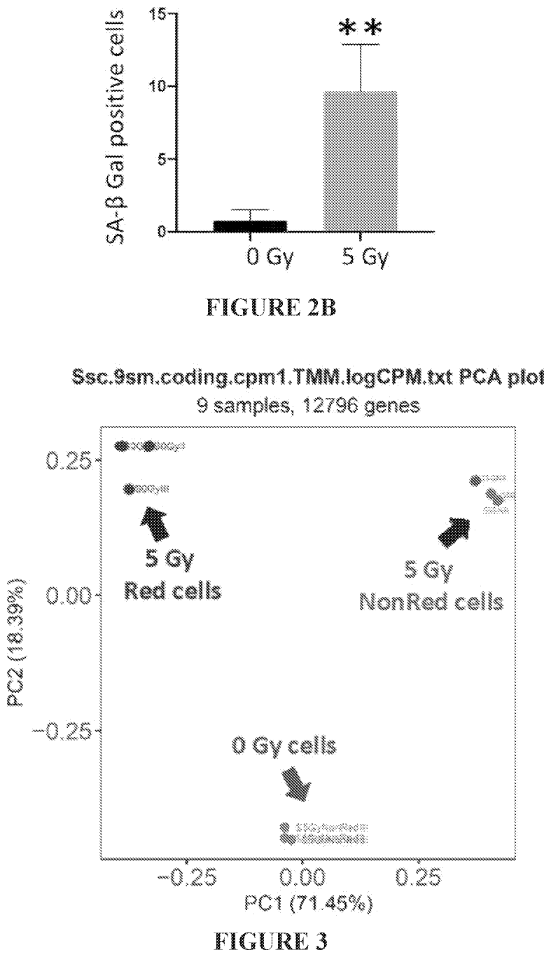

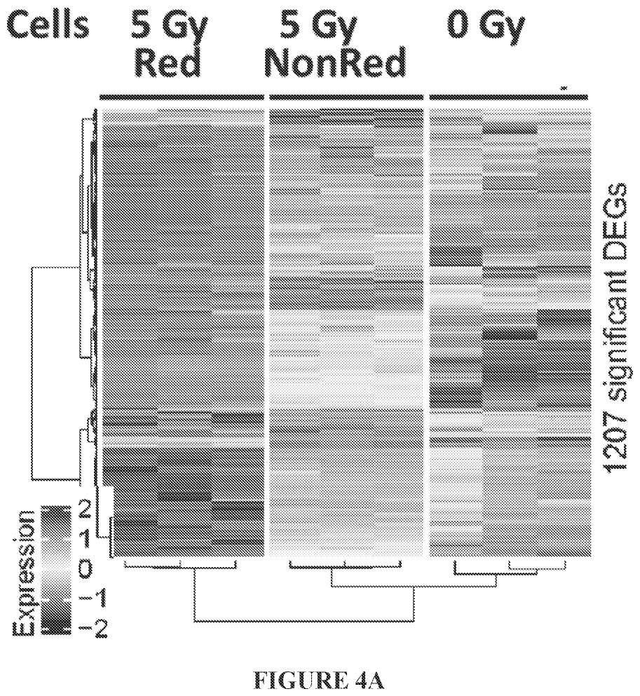

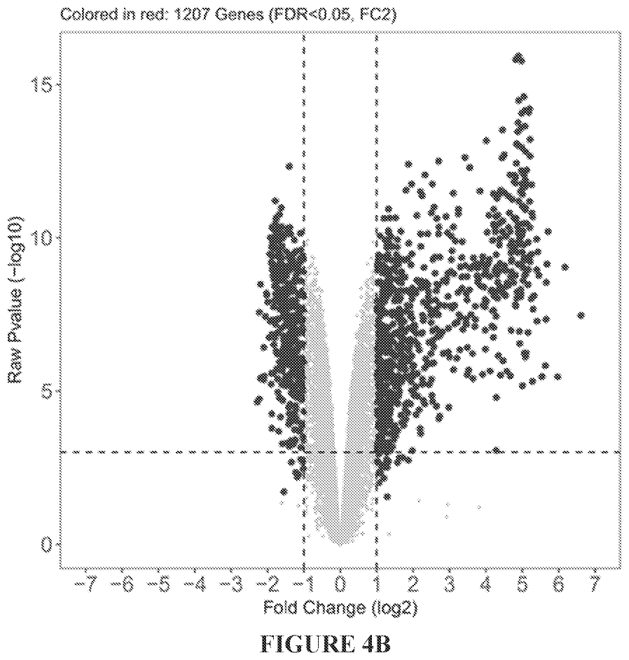

Results: It has been shown that 5 Gy irradiation of tdTOMp16+ cells in logarithmic phase growth induces ˜9% cellular senescence after 10 days (FIG. 1). A pure population of red tdTOMp16+ cells was isolated by fluorescence activated cell sorting (FACS). Sorted single irradiated red cells, irradiated non-red cells and control nonirradiated cells placed into 96-well plates were monitored over four-weeks for cell division (FIG. 2A). Irradiated senescent red cells did not divide, 0/960 cells divided, while nonirradiated and irradiated non-red cells did divide (FIG. 2A). Irradiated non-red cell division was predictably less than the control nonirradiated cells (FIG. 2A). RNA-seq was performed with triplicates of each of the three cell groups after extracting the total RNA from: 1) control nonirradiated cells; 2) sorted irradiated tdTOMp16+ red; and 3) irradiated, but non-red cells (FIG. 3). RNA quality as confirmed by RNA integrity number and was constant in all 9 samples. After RNA-seq, the data was analyzed for principal component analysis (PCA) (FIG. 3) and separation of the 3 cell populations, and it was shown that the three biological repeats were clustered together. Comparing 5 Gy irradiated senescent red cells with 5 Gy non-red non-senescent cells, it was found that 1174 genes were differentially expressed of which 782 were upregulated and 392 were downregulated. In hierarchic clustering of differentially expressed genes by heatmap analysis, the three groups showed profoundly up and downregulated genes in red senescent cells (FIG. 4A-FIG. 4B). A customized search for the gene enrichment analysis showed upregulation in senescent cells of three gene categories: including those associated with chemotaxis, phagocytosis, and fibrosis (overall upregulated).

The ranking of the top 10 genes was analyzed according to their experimental fold change (Table 1). Upregulated genes in irradiated red senescent cells were compared to irradiated non-red cells and a top upregulated gene for the tyrosine-kinase Fgr, the expression of which was 72-fold elevated was discovered. This elevation of Fgr was of greater magnitude than the level observed with any other chemotaxis or phagocytosis related transcript or any downregulated gene (Table 1).

TABLE 1
Top upregulated overall genes in irradiated red senescent cells compared
to irradiated non-red cells by RNA-seq, compared to upregulated
genes for chemotaxis, phagocytosis, and downregulated genes.

| Top upregulated | | | | | | Phagocytosis & | | | Top downregulated | | |
|---|---|---|---|---|---|---|---|---|---|---|---|
| genes, overall | | | Chemotaxis | | | engulfment | | | genes, overall | | |
| Gene | Expr. Fold | | Gene | Expr. Fold | | Gene | Expr. Fold | | Gene | Expr. Fold | |
| symbol | change | | symbol | change | | symbol | change | | symbol | change | |
| Fgr | 72.102 | [symbol] | Ccl12 | 43.08 | [symbol] | Fcgr1 | 49.799 | [symbol] | Plac9b | −4.68 | [symbol] |
| Osm | 62.782 | [symbol] | Ccl3 | 34.735 | [symbol] | Fcgr2b | 36.807 | [symbol] | Cenpu | −4.675 | [symbol] |
| Cd48 | 52.261 | [symbol] | Ccr1 | 31.374 | [symbol] | Fcgr3 | 34.048 | [symbol] | Htr7 | −4.564 | [symbol] |
| Bcl2a1a | 51.243 | [symbol] | Ccl4 | 30.903 | [symbol] | Itgam | 33.679 | [symbol] | Epha3 | −4.563 | [symbol] |
| Rasal3 | 50.49 | [symbol] | Ccl6 | 29.825 | [symbol] | Trem2 | 32.671 | [symbol] | Phf19 | −4.307 | [symbol] |
| Ebi3 | 50.359 | [symbol] | Cxcr4 | 24.617 | [symbol] | Cd36 | 29.922 | [symbol] | Pcdh11x | −4.263 | [symbol] |
| Fcgr1 | 49.799 | [symbol] | Ccrl2 | 12.41 | [symbol] | Nckap1l | 28.679 | [symbol] | Exo1 | −4.161 | [symbol] |
| Ptpro | 46.493 | [symbol] | Ccl9 | 5.966 | [symbol] | Rac2 | 28.194 | [symbol] | Spag5 | −4.066 | [symbol] |
| Tal1 | 43.958 | [symbol] | Cxcl2 | 5.309 | [symbol] | Msr1 | 27.424 | [symbol] | Pif1 | −4.04 | [symbol] |
| Ccl12 | 43.08 | [symbol] | Ccl5 | 3.832 | [symbol] | Bin2 | 25.913 | [symbol] | Ska1 | −3.973 | [symbol] |

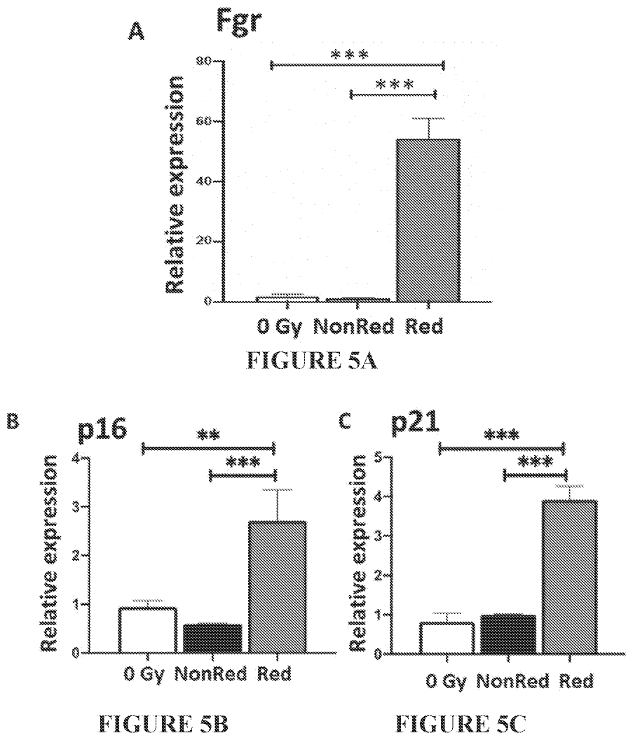

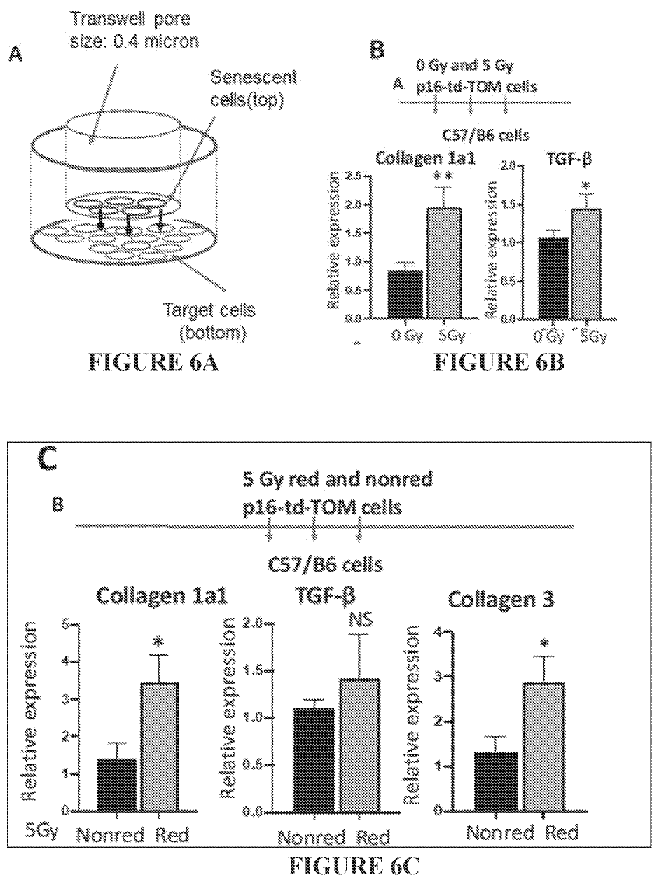

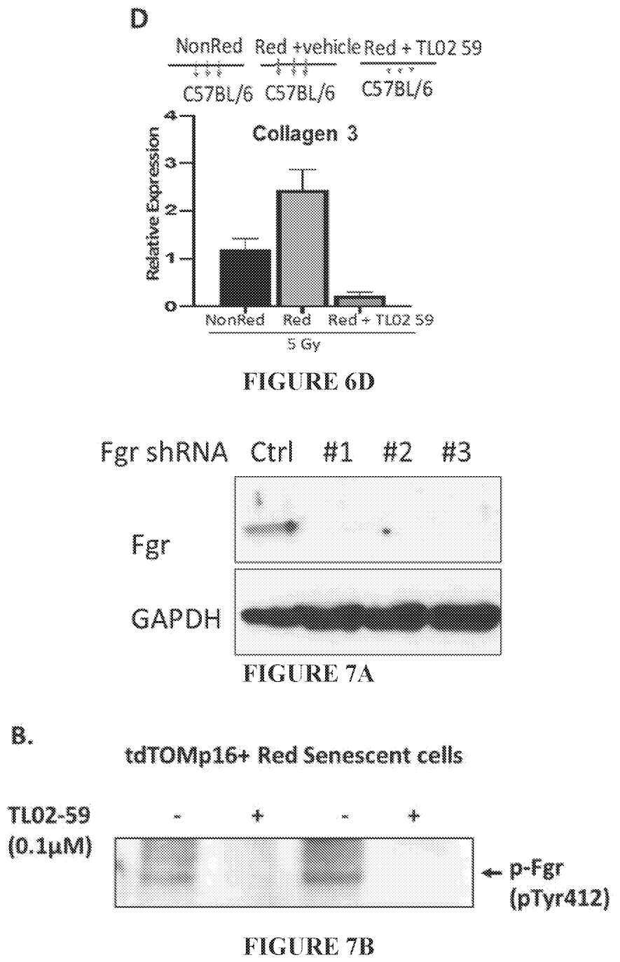

It was also confirmed that the RIS cells showing upregulation of Fgr RNA by heat map also had higher RNA levels. Validation of RNA for p16 and p21, as well as Fgr was carried out (FIG. 5A-FIG. 5C). To establish that irradiated tdTOMp16+ senescent cells induce profibrotic genes, a C57BL/6 target cell line, and a noncontact transwell co-culture system was used (FIG. 6A). Irradiated red cells were compared to 0 Gy or irradiated non-red cells. Sorted red cells, were placed on the top of each well and C57/B6 mouse stromal target cells were plated at the bottom well and observed for two weeks. There was significant induction of collagen 1al and collagen 3 in C57/B6 target cells by RIS red cells (FIG. 6B-FIG. 6C). TGF-β was also upregulated (FIG. 6B, FIG. 6C). Addition of the small molecule specific inhibitor of Fgr TL02-59 reduced the induction of collagen 3 by RIS red cells. (FIG. 6D).

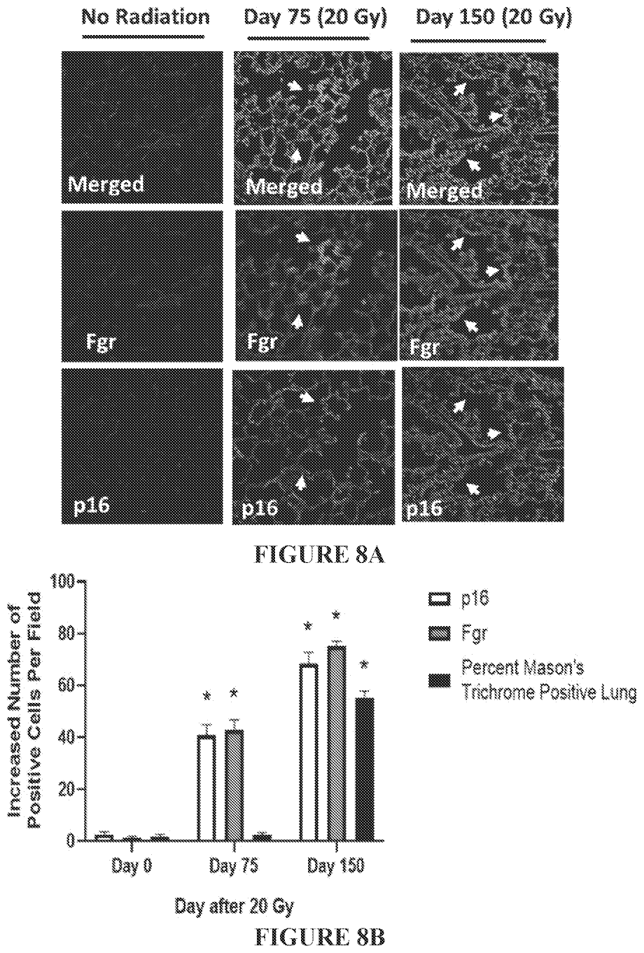

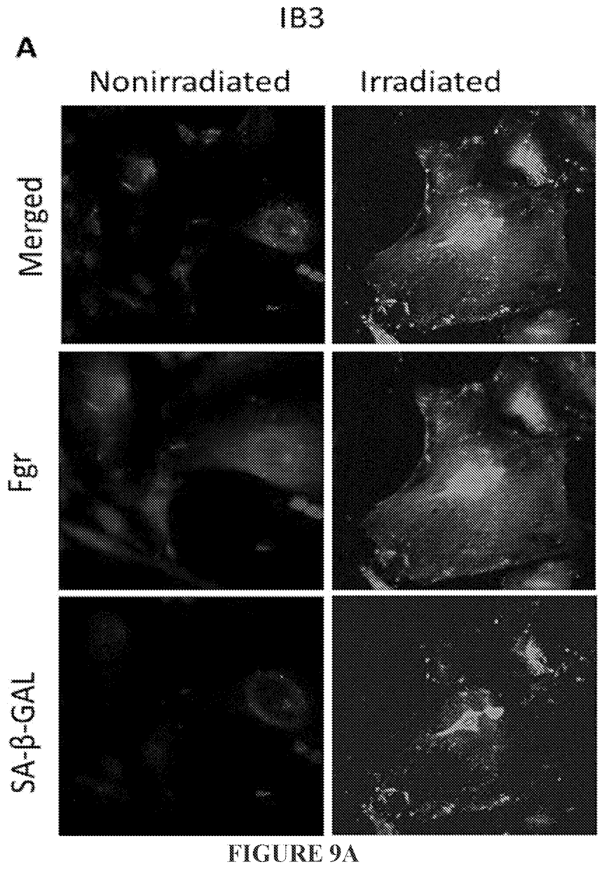

FIG. 7A-FIG. 7B shows successful knock down of Fgr by shRNA in tdTOMp16+ cells in vitro. The data show that Fgr+ senescent cells are clearly detectable in the irradiated lung prior to detection of pulmonary fibrosis (FIG. 8A-FIG. 8B).

### Experimental

Specific Method 1: Establish the effects on target cells of downregulation of Fgr in RIS cells.

Sub-Method IA: Establish that Fgr silencing by shRNA in RIS tdTOMp16+ cells reduces induction of biomarkers of fibrosis in target cells. Construction of stable inducible cell line expressing Fgr shRNA can be carried out as follow. Subclonal lines of tdTOMp16+ stromal cell line that stably express TetR (repressor) are generated using the antibiotic Blasticidin.

Resistant cell clones are then selected, and multiple clones tested for TetR expression by immunoblotting the best TetR-positive clone expanded. Tet-on cell lines with inducible Fgr shRNA expression are generated next. TetR-positive cells are then transfected with Invitrogen™ BLOCK-^{iTTM} Inducible H1 RNAi Entry Vector Kit for shRNA expression, and Blasicidin and Zeocin resistant clones selected. Several cell clones are expanded and screened by immunoblotting for Tetracycline-inducible knockdown of Fgr by shRNA. A stably inducible tdTOMp16+ cell line is isolated using lentiviral shRNAs plasmids specific for Fgr or a control scrambled shRNA and doxycycline (doxy) added to knockdown Fgr in radiation induced red tdTOMp16+ cells. In a non-contact transwell coculture system, irradiated cells are plated on the top of the transwell, and the target C57BL/6 mouse marrow stromal target cell line at the bottom and it can be established that RIS cells with silenced Fgr do not induce fibrosis markers in the target cells (doxy added pre or post-sorting of red senescent cells). The effects of Fgr silencing using are compared by: 1) irradiated and sorted red tdTOMp16+ cells, 2) irradiated sorted non-red tdTOMp16+ cells, and nonirradiated tdTOMp16+ cells by both (a) immunostaining for profibrotic genes in target cells including collagen I-IV and smooth muscle actin and by real time qPCR for fibrosis related genes TGF-β1, CTGF, TNF, NF-κB, IL-1, and Col1-Col6 [19], and other inflammatory and stress response proteins by Luminex assay. It can be established that the antifibrotic effects of silencing Fgr can be reversed by removal of doxycycline which will stop the effects of shRNA.

Sub-Method 1B: Identify the SASP factor(s) released from RIS cells that mediate the induction of profibrotic biomarkers in target cells in transwells. Proteomics are carried out with the conditioned medium from RIS cells compared to that from cell populations of RIS cells after shRNA knockdown of Fgr.

Sub-Method 1C: Establish the antifibrotic effects of small molecule Fgr inhibitor TL02-59 on RIS in transwells in vitro. The concentration and time dependent reduction of biomarkers of fibrosis in target cells is established by adding TL02-59 to senescent tdTOMp16⁺ cells in transwells.

Cells can be irradiated in vitro using a Cesium-137 JL Shepherd Model 68 irradiator at 3 Gy per minute.

Two-way ANOVA models can be used where treatment and time and their interaction are factors followed by Tukey's multiple comparison tests.

Summary: Fgr is a member of the tyrosine kinase family and its potential profibrotic functions have been described. It can be shown that shRNA shut off production of Fgr in senescent cells and abrogate their capacity to induce collagen 1A, collagen 3, and TGF-β3 in target cells in transwells. It is believed that shRNA mediated knockdown in RIS cells will shut off production of secreted profibrotic protein products, and silence profibrotic genes in target cells. The methods are expected to identify several components of the SASP, which are pro-fibrotic. A dose response curve on the number of senescent cells needed in the top of the transwell admixed with decreasing numbers of non-irradiated cells can be performed to determine the minimum number of senescent cells required to induce profibrotic genes.

Specific Method 2: Identify the phenotype(s) of Fgr+ senescent cells in the irradiated lungs that appear before detectable fibrosis.

Sub-Method 2A. A comparison of the levels of Fgr in sorted red radiation-induced senescent cells from lungs over time can be made as follow. Red RIS lung cells are sorted from tdTOMp16+ mice explanted from 20 Gy thoracic irradiated and control tdtOMp16+ mice at day 75 (early) and days 150-180 (late), and senescent (red) cells removed. RNA-seq can be used to compare results with red cells in overlying thoracic skin and cell line in vitro. RIS cells can be removed at days 0, 25, 50, 75, 100, 125, and 180 after 20 Gy vs. those sorted from overlying thoracic skin vs. bone marrow stromal cell line from tdTOMp16+ mice by RNA-seq for elevated levels of Fgr and other components of this SASP. Whole RNA-seq can be used in sorted cells. 10 mice per time point for a total of 70 male and 70 female mice=total 140 mice can be used.

Sub-Method 2B: Establish the phenotype(s) of irradiated lung cells that produce elevated levels of Fgr after irradiation: (endothelial vs. epithelial vs. monocyte/macrophage). Lung cells have been sorted for endothelial (CD31) positive cells. Epithelial cells (CD326 or EPCAM) positive cells have also been sorted. Other cell phenotypes including immunocytes can be sorted. RNA-seq is performed on RIS cells from each phenotype and the level of Fgr compared with that of in vitro irradiated cell line. If numbers are small, single cell RNA-seq is carried out on individual RIS cell phenotypes. Cells can be sorted from 0 Gy or 20 Gy irradiated mice. Number of mice at specific times for this method can be 2 radiation doses, 20 mice×3 replicates, 120 males and 120 female mice=240 total mice.

Cells can be irradiated in vitro using a Cesium-137 JL Shepherd Model 68 irradiator at 3 Gy per minute. Mice can be thoracic irradiated using 6 MV photons delivered at 600 monitor units per minute by a Varian True Beam Irradiator. The irradiation field can be 2 cm×40 cm with an SSD of 100 cm.

Two-way ANOVA models can be used where treatment and time and their interaction are factors followed by Tukey's multiple comparison tests. For fibrosis studies, a sample size of 7 per group can provide 83% power to detect the expected difference of 1.7 effect size at 0.05 significance level using the two-sided two-sample t-test or Wilcoxon rank sum test where appropriate.

Summary: It is believed that that senescent cells of both endothelial and epithelial phenotype can appear in the lung as early as 50 days after 20 Gy thoracic irradiation and will produce Fgr and precede fibrosis. The levels of RNA in senescent cells appearing at different time points at day 75, day 100, or day 120 after 20 Gy thoracic irradiation can be compared by RNA-seq and single cell RNA-seq.

Specific Method 3: Establish that administration of the Fgr inhibitor TL02-59 to 20 Gy thoracic irradiated C57BL/6 male and female mice ameliorates/prevents RIPF.

Sub-Method 3A. The reduction of fibrosis in the lungs of TL02-59 treated tdTOMp16+ mice after 20 Gy Thoracic irradiation can be quantified as follow. Since it is known that at day 75 these mice display RIS in lungs, but no detectable fibrosis and there is fibrosis at day 150 (FIG. 8A-FIG. 8B), the Fgr inhibitor TL02-59 can be given by daily gavage starting at day 120 for 21 days at a dose of either daily 1 or 10 mg/kg in 10% DMSO plus 90% corn oil. Control mice receive vehicle only. Pharmacokinetics of TL02-59 in mice demonstrated that TL02-59 is orally bioavailable and displays a plasma half-life of 6 h in mice, making it an attractive candidate mitigator for in vivo evaluation. After 21 days, the mice are sacrificed and lung tissue collected for mRNA, protein, and histological analyses of RIPF using known methods. All animal studies are to be carried out in male and female mice, and conducted in accordance with the guidelines and regulations established by the University of Pittsburgh Institutional Animal Use and Care Committee (approved protocol 18022000). This method can require 30 male and 30 female mice (n=15). Total mice for all aims and all experiments will be 480.

Sub-Method B: Measure Fgr in lung cell phenotypes after TL02-59 treatment. The volume of senescent cells and magnitude of fibrosis can be quantitated by immunohistochemistry in serial sections using senescence markers p16 and SA-β-gal and Masson's Trichrome stain for fibrosis in each cell phenotype at day 150.

Mice will be thoracic irradiated using 6 MV photons delivered at 60) monitor units per minute by a Varian True Beam Irradiator. The irradiation field can be 2 cm×40 cm with an SSD of 100 cm.

Two-way ANOVA models can be used where treatment TD02-59 vehicle or radiation dose are factors followed by Tukey's multiple comparison tests. For fibrosis studies, a sample size of 7 per group can provide 83% power to detect the expected difference of 1.7 effect size at 0.05 significance level using the two-sided two-sample t-test or Wilcoxon rank sum test where appropriate.

Summary: TL02-59 is remarkably selective for Fgr over other kinases. TL02-59 treatment inhibits Fgr with partial inhibition observed at 0.1-1 nM and complete inhibition above 10 nM. In contrast, other similar kinases require (100-1000 nM) that is a 10 to 100 fold greater dose required for inhibition. In a 10 nM concentration, TL02-59 inhibits only Fgr. It is believed that the TL02-59 drug given daily starting at day 120 after 20 Gy thoracic irradiation will optimally reduce fibrosis.

Overall Summary: In the p16^{tdTOM+} mouse strain, senescent cells have been induced by irradiation, sorted and purified as red senescent cells. For the first time, it has been demonstrated by RNA_{SEQ} that RIS cells produce a unique protein (Fgr), which induces biomarkers of irradiation pulmonary fibrosis in vitro in target cells in transwell cultures. It was shown that Fgr is present in senescent cells in the thoracic irradiated lung significantly before detection of radiation-induced pulmonary fibrosis (RIPF). Also, identified was a small molecule inhibitor of Fgr (TL02-59), which is now established as a new mitigator of RIPF. The experiments herein have and will establish that irradiation-induced senescent cells in the lung produce Fgr that induces fibrosis, and that Fgr is inhibited by a targeted small molecule. These experiments will identify the mechanism of action of Fgr, the phenotype of lung RIS cells that produce Fgr and will provide both a new target and new inhibitor drug for amelioration and prevention of RIPF.

### Example 2: Irradiated Human Morrow Stromal and Lung Epithelial Cell Lines Become Senescent and Co-Express Fgr

Method: Human bone marrow stromal cell line KM101, and human bronchial epithelia cell line 1B3, were irradiated with 5 Gy by Cesium Gamma Cell dose rate 300 cGy/min. 8 days post-radiation the cells were fixed and stained for Fgr antibody (Santacruz #sc-74542), and SA-beta-GAL staining kit (CellEvent™ Senescence Green Flow Cytometry Assay Kit from Fisher Scientific #C10841). The statistical analysis was done by non-parametric t-test.

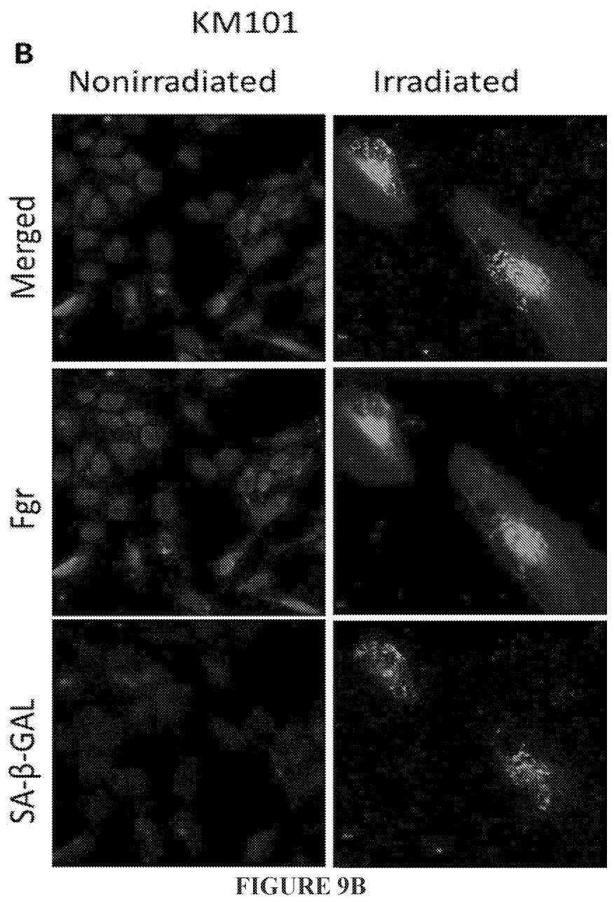

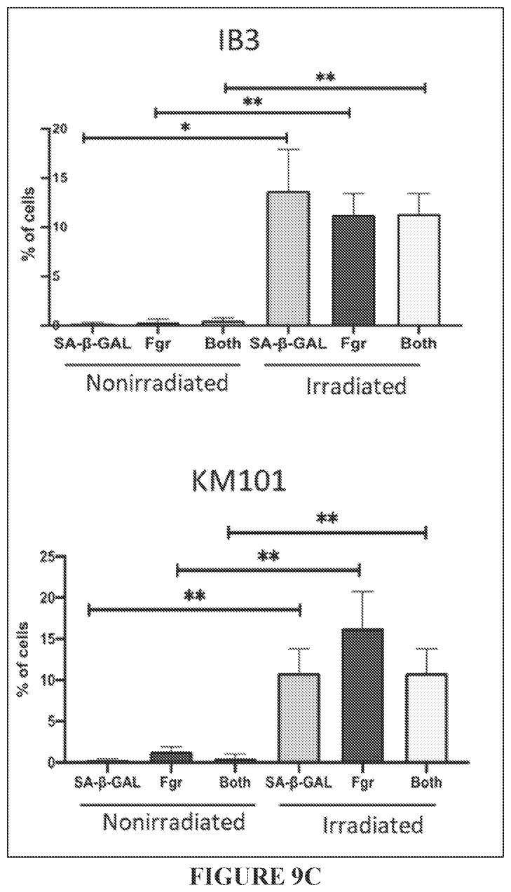

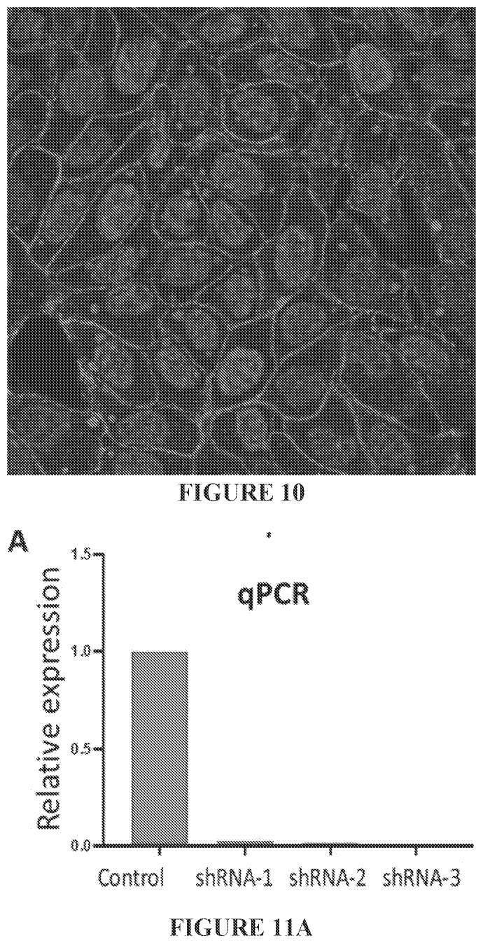

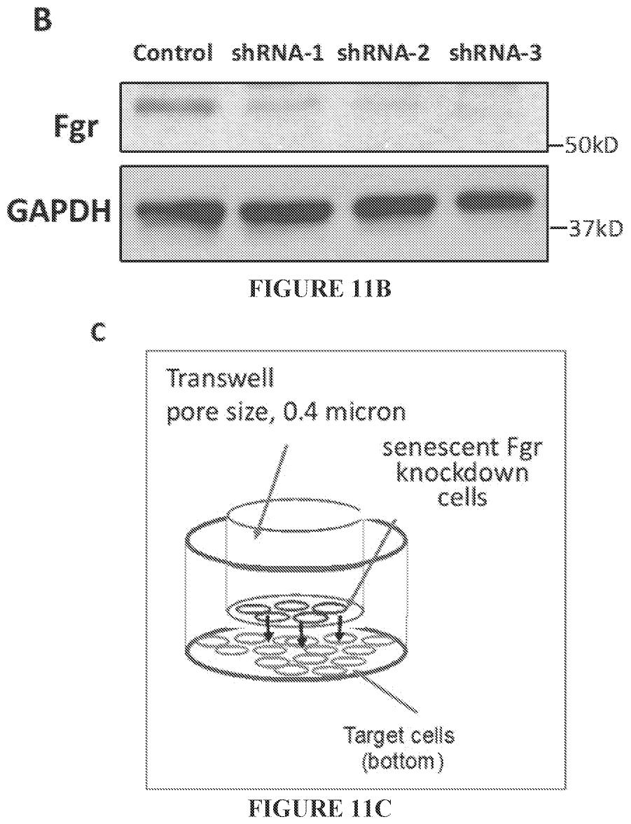

FIG. 9A-FIG. 9C shows upregulation of Fgr in radiation induced senescent human cell lines. FIG. 9A shows human bronchial epithelial cell line IB3, and FIG. 9B shows human bone marrow stromal cell line KM101 were irradiated (5 Gy) and stained with SA-beta-GAL staining kit and Fgr antibody. FIG. 9C shows a graph represents quantitative analysis (%) for SA-beta-GAL and Fgr positive cells in IB3 (top) and KM101 (bottom) cell lines. The statistical analysis was done by non-parametric t-test. N=3, *, p<0.05; **, p<0.01.

FIG. 10 shows immunofluorescent staining of Fgr (Green) in human cell line RT4 shows localization to plasma membrane. Nuclei are in blue.

### Example 3: Silencing of Fgr in Senescent Cells Inhibits the Fibrotic Response on

C57BL/6 Target Cell Line Fgr shRNA experiment: For Fgr silencing, mouse specific Fgr shRNA (TRCN0000023471) was purchased from Sigma and the mouse bone marrow stromal tdTOMp16+ cell line was transfected using Lipofectamine 3000 (Thermo Fisher Scientific, L3000-008). Stable tdTOMp16 Fgr-knockdown cell line was created by selecting the cells for puromycin resistance (4 ug/ml). For the evaluation of shRNA efficacy total RNA was extracted using TRIzol (Thermo Fisher Scientific, Cat. No. 15596018) and shRNA knockdown was evaluated by qPCR (From Thermo Fisher Scientific, TaqMan Assay #Mm00438951_m1) and western blotting was performed using Fgr antibody (Santa Cruz Biotechnology, Inc Catalogue No. sc-74542).

Transwell experiment: Control and Fgr knockdown stable cell lines were irradiated with 5 Gy by Cesium Gamma Cell dose rate 300 cGy/min. Ten days after radiation, red senescent and non-red non-senescent cells were sorted using MoFlo Astrios Cell Sorter (Beckman Coulter). Senescent and non-senescent cells were plated on the top well of the transwell with a 0.4 mm pore size (Coster, Cat. No. 3412) and the target C57BL/6 cell line for the evaluation of fibrotic markers was plated at the bottom. 2 weeks later total RNA was extracted from the target C57BL/6 cells and evaluated by qPCR for Collagen 3 (Mm01254476_m1) and TGF-beta (Mm01191861_m1). GAPDH (Mm99999915_g1) was used as the loading control.

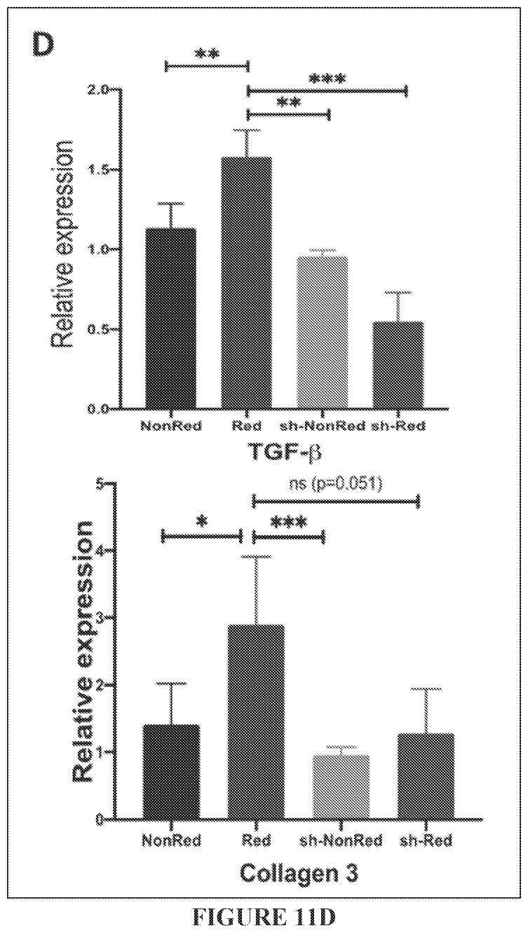

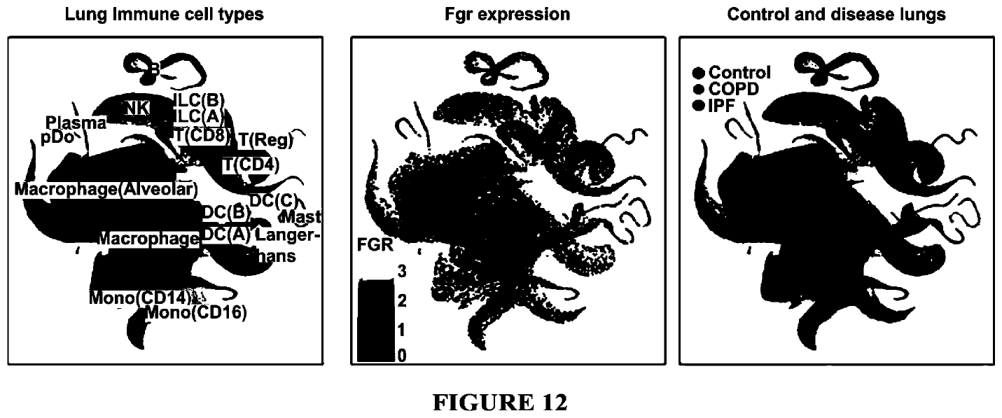

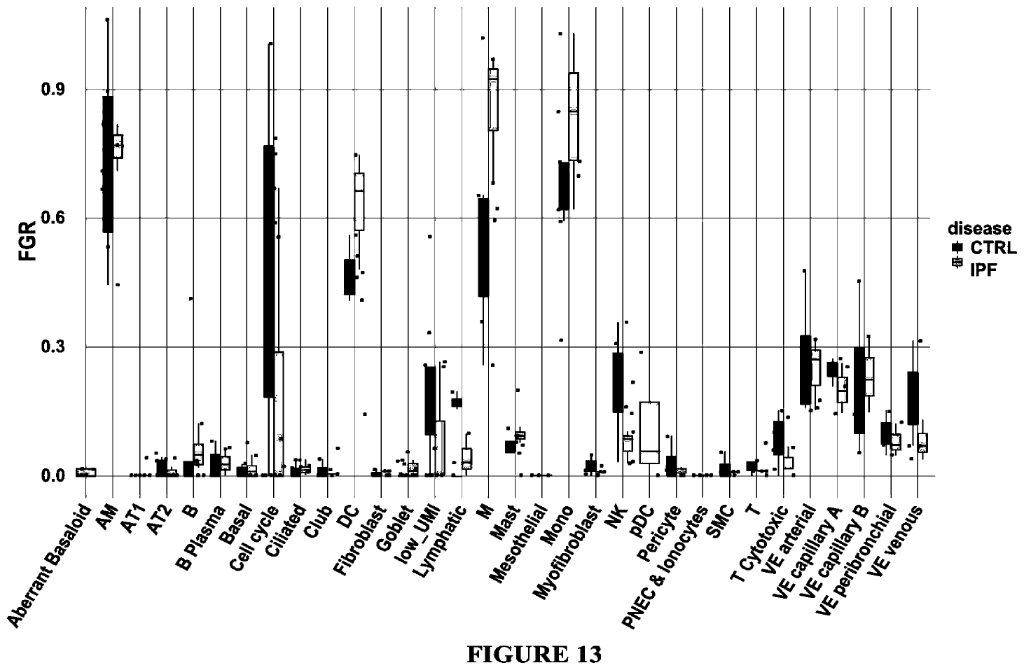

FIG. 11A-FIG. 11D shows Fgr knockdown in irradiated tdTOMp16+ senescent cells reduces its profibrotic potential. Expression of Fgr was silenced by shRNAs and evaluated by qPCR (FIG. 11A), and Western blot (FIG. 11B). Both control and shRNA knockdown cells were sorted for irradiated red senescent and non-red cells and plated on the top well of transwells and cocultured with C57BL/6 stromal cells at the bottom (FIG. 11C). The expression of Collagen3 and TGFβ in the C57BL/6 stromal cells was evaluated by qPCR (FIG. 11D). N=3, * p<0.05; **, p<0.01; ***, p<0.001.

### Example 4

Survivors of the acute radiation syndromes (ARS) present with delayed effects of acute radiation exposure (DEARE), which include radiation-induced pulmonary fibrosis (RIPF) (Unthank J L et al. Health Phys, 2015, 109(5), 511-521). Radiation-induced pulmonary fibrosis is also a side effect of clinical radiotherapy for lung cancer patients (Perez J R et al. Sci Rep. 2017. 7(1), 9056). Senescent cells have been implicated in radiation-induced pulmonary fibrosis as well as in idiopathic pulmonary fibrosis; senolytic drugs have been shown to improve lung function in experimental fibrosis models (Schafer M J et al. Nat Commun, 2017, 8, 14532; Hohmann M S et al. Am J Respir Cell Mol Biol, 2019, 60(1), 28-40; Kalash R et al. Radiat Res. 2013, 180(5), 474-490; Sessions G A et al. FASEB J, 2019, 33(11), 12364-12373; Baar M P et al. Cell, 2017, 169, 132-147; Gorgoulis V et al. Cell, 2019, 179: 813-825; Hernandez-Segura A et al. Current Biology, 2017, 27, 2652-2660; Schafer M J et al. Nature Communications, 2017, 8, 14532). The molecular mechanism by which radiation induced senescence (RIS) induces fibrosis is unknown (Soysouvanh F et al. Int J Radiation Oncol Biol Phys, 2020, 106(5), 1017-1027; Baar M P et al. Cell. 2017, 169, 132-147; Gorgoulis V et al. Cell. 2019, 179: 813-825; Hernandez-Segura A et al. Current Biology, 2017, 27, 2652-2660; Schafer M J et al. Nature Communications, 2017, 8, 14532).

Herein, a tyrosine kinase, Fgr, was identified by RNA-seq analysis of sorted tdTOMp16+ mouse RIS cells, which is significantly upregulated to a level greater than that of any other transcript including those associated with chemotaxis or phagocytosis. RIS cells induce biomarkers of fibrosis in separated target cells in transwell cultures in vitro. Fgr is increased in RIS cells in situ in irradiated mouse lung before the appearance of fibrosis. The central hypothesis is that upregulation of Fgr tyrosine kinase in RIS precedes and induces radiation pulmonary fibrosis. Furthermore, genetic or pharmacologic inhibition of Fgr can mitigate radiation-induced pulmonary fibrosis.

Genetic inhibition of Fgr by shRNA knockdown, deletion of Fgr in knock-out mouse, and pharmacologic inhibition of Fgr by a specific small molecule TL02-59 can prevent radiation-induced pulmonary fibrosis. To achieve this, a primary stromal cell line from tdTOMp16+ mice (Sessions G A et al. FASEB J, 2019, 33(11), 12364-12373) was established and a pure population of radiation induced p16 expressing red senescent cells was sorted by fluorescence activated cell sorting (FACS). RNA-seq was performed on this pure population of RIS cells and it was shown that gene expression profiles of RIS cells are distinct from both irradiated non-senescent and non-irradiated cells. It was shown that RIS stromal cells as well as RIS cells isolated from radiation-induced pulmonary fibrosis mouse lungs can induce biomarkers of fibrosis in separated target cells in transwell cultures. Furthermore, shRNA knockdown as well as Fgr inhibitor TL02-59 have been shown to abrogate the induction of fibrotic biomarkers in target cells. C57BL/6 tdTOMp16+ mice develop pulmonary fibrosis at 180 days after 20 Gy thoracic irradiation, and from single cell RNA-seq (scRNAseq) analysis it was shown that Fgr is upregulated in lung senescent cells which appear in significant numbers in the lungs before the occurrence of fibrosis. Fgr positive senescent cells were isolated by FACS from irradiated lungs, the profibrotic SASP component secreted by them were identified, and their phenotype (epithelial, endothelial, immunocyte) was determined. It was established that inhibition of Fgr by administration of TL02-59 to mice prevents radiation pulmonary fibrosis.

Specific Aim 1: Elucidate the mechanism by which downregulation of Fgr in RIS cells reduces their capacity to induce biomarkers of radiation fibrosis. 1A: Establish that shRNA inhibition of Fgr in RIS cells or cells isolated from tdTOMp16+ Fgr−/− mouse RIPF lungs, fail to induce biomarkers of fibrosis in target cells. 1B: Identify the SASP factor(s) released from RIS cells that mediate the induction of profibrotic biomarkers in target cells in transwells. 1C. Establish that Fgr knockdown in RIS cells by shRNA or Fgr−/− cells isolated from Fgr-KO mice do not secrete profibrotic SASP components from Aim 1B.

Specific Aim 2: Determine the phenotype(s) of Fgr positive RIS cells, which appear in the irradiated lungs prior to and during the onset of fibrosis. 2A: Identify the lung cell types that express Fgr and senescent biomarkers prior to lung fibrosis by single-cell RNA-seq. 2B: Establish the phenotype(s) of irradiated lung cells producing elevated levels of Fgr after irradiation by cell sorting. 2C. Demonstrate upregulation of Fgr in RIPF tissue of human patients.

Specific Aim 3: Demonstrate that blocking of Fgr in vivo reduces pulmonary fibrosis. 3A: Quantitate the reduction of pulmonary fibrosis of thoracic irradiated tdTOMp16+ mice with TL02-59 treatment. 3B: Measure Fgr expression in lung cell phenotypes after TL02-59 treatment. 3C: Quantitate the reduction of pulmonary fibrosis of C57BL/6 Fgr−/− mice after thoracic irradiations.

These experiments can establish the role of senescence in radiation-induced pulmonary fibrosis and identify a new pulmonary radiation countermeasure.

Background/Significance: There is increasing evidence that ionizing irradiation induced senescence is associated with fibrosis (Soysouvanh F et al. Int J Radiation Oncol Biol Phys, 2020, 106(5), 1017-1027; Schafer M J et al. Nature Communications, 2017, 8, 14532), and that administration of a senolytic drug to fibrosis-prone mice can reduce fibrosis (Kalash R et al. Radiat Res. 2013, 180(5), 474-490; Sivananthan A et al. Radiat Res, 2019, 191, 139-153; Baar M P et al. Cell. 2017, 169, 132-147; Gorgoulis V et al. Cell. 2019, 179: 813-825; Hernandez-Segura A et al. Current Biology. 2017, 27, 2652-2660; Schafer M J et al. Nature Communications, 2017, 8, 14532). The senescence associated secretory phenotype (SASP) of irradiated cells is distinct from that observed in other causes of senescence (Hernandez-Segura A et al. Current Biology. 2017, 27, 2652-2660). Using the tdTOMp16+ mouse strain (Sessions G A et al. FASEB J, 2019, 33(11), 12364-12373), senescent cells were induced by irradiation, and red senescent cells were sorted and purified. RNA-seq demonstrated that RIS cells produce a unique protein (Fgr), which induces biomarkers of irradiation pulmonary fibrosis in vitro in target cells in transwell cultures. It was shown that Fgr is induced in senescent cells in the thoracic irradiated mouse lung significantly before detection of radiation-induced pulmonary fibrosis (RIPF) and that Fgr is induced in the lungs of human patients with radiation-induced pulmonary fibrosis. The idiopathic pulmonary fibrosis (IPF) lung atlas shows that Fgr is induced in fibrotic human lungs (Uhlen M et al. Science, 2015, 347(6220), 1260419). A small molecule inhibitor of Fgr (TL02-59), which is a potential mitigator of radiation-induced pulmonary fibrosis, was identified.

A target for inhibiting radiation-induced pulmonary fibrosis was discovered in a purified and isolated population of irradiation induced senescent cells and its relevance was confirmed in the lungs of both mouse and human radiation-induced pulmonary fibrosis. In cell lines and in lungs of tdTOMp16+ mice, a product of irradiation induced senescent cells, Fgr, was discovered, which induces biomarkers of fibrosis in vitro and is present in RIS cells in the lungs in vivo. It was shown that shRNA knockdown of Fgr in cells, or by adding a specific Fgr inhibitor drug, TL02-59 to RIS cells, inhibits induction of biomarkers of fibrosis in target cells. Fgr-knockout mice (Fgr−/−) were obtained and used.

The experiments can establish that irradiation-induced senescent cells in the lung produce Fgr that induces fibrosis, and that Fgr can be inhibited by a targeted small molecule. These studies can identify the mechanism of action of Fgr, the phenotype of lung RIS cells that produce Fgr, and can provide both a target and inhibitor drug for amelioration and prevention of radiation-induced pulmonary fibrosis.

Approach: There is currently no direct proof that RIS cells induce radiation pulmonary fibrosis. Using a tdTOMp16+ mouse bone marrow stromal cell line, a pure population of red tdTOMp16+ (Sessions G A et al. FASEB J, 2019, 33(11), 12364-12373) senescent cells were sorted by FACS at 10 days after 5 Gy irradiation and RNA-seq was performed. It was confirmed that there is enrichment of SASP components and a 72-fold upregulated level of a transcript was identified for a profibrotic tyrosine kinase, Fgr. A non-contact transwell co-culture system was used to establish the antifibrotic effects of shRNA knockdown of Fgr, Fgr deletion using cells from Fgr-knockout mice, and adding an inhibitor of Fgr. In a coculture system, the RIS cells are on the top chamber of transwells and target cells on the bottom (separated by 0.4-micron filter), the components of SASP from the RIS cells that induce fibrosis can be identified. Fgr positive senescent cells were identified in the lungs of 20 Gy thoracic irradiated mice in vivo prior to the appearance of fibrosis and senescent cells were isolated from mouse RIPF lungs. Both genetic and pharmacologic inhibition of Fgr can be used to abrogate the biologic effects of RIS in vitro and RIPF in vivo. shRNA knockdown of Fgr in irradiated senescent cells as well as irradiated senescent Fgr−/− cells (plated on top in a transwell culture system) over a monolayer of C57BL/6 mouse target cells (bottom) can be used to assess their capacity to induce biomarkers of fibrosis and the results can be compared with non-senescent cells. Using the small molecule inhibitor of Fgr, TL02-59, which selectively inhibits Fgr (Du G et al. Cell Death Dis, 2020, 11(2), 118) and using Fgr−/− RIS cells isolated from irradiated Fgr-KO mouse, the Fgr mediated and secreted factors can be identified by cytokine array of conditioned medium from RIS cells. It can be determined which mediators of fibrosis are induced in transwells in vitro and factors which are absent from the same RIS cells after Fgr knockdown or Fgr deletion can be identified. TL02-59 can be added to transwell cultures and conditioned medium can be analyzed for the absence of profibrotic signals. The phenotype(s) of senescent cells in 20 Gy thoracic irradiated tdTOMp16+ mouse lungs, which show upregulated Fgr, can be determined. By single-cell RNA-seq (scRNAseq), it was shown that there is Fgr upregulation in mouse RIPF lungs in immune cells. scRNA-seq of mouse lung cells prior to the appearance of fibrosis after 20 Gy thoracic irradiation can be performed to identify cell types that show induced Fgr. Transwell experiments can establish that RIS lung cells produce profibrotic factors and these factors can be identified by performing cytokine array of SASP using conditioned media and the cell lysates. It can be established that the Fgr-KO mouse is resistant to radiation-induced pulmonary fibrosis and that TL02-59 inhibits fibrosis in vivo by reducing levels of profibrotic products that are produced by Fgr positive RIS cells. TL02-59 can be administered to tdTOMp16+ mice at serial times after 20 Gy thoracic irradiation when senescent cells are first detected and the time course and degree of amelioration of fibrosis can be established.

Results: It was shown that 5 Gy irradiation of tdTOMp16+ cells in logarithmic phase growth induces ˜9% cellular senescence after 10 days (FIG. 1). FIG. 1 shows a selection of optimum cell density at the time of the radiation, radiation dose, and days after radiation when maximum % of tdTOMp16+ senescent cells are detected. Cells at 50% density, 5 Gy irradiated, and harvested at day 10 achieved 9% RIS cells (n=4; *, p=<0.05; **, p=<0.01, calculated by Two-way ANOVA). Significance was detected only with 5 and 7.5 Gy for 9 and 10 days. To reduce toxicity, 5 Gy was chosen.

A pure population of red tdTOMp16+ cells was isolated by fluorescence activated cell sorting (FACS) and senescence was confirmed by their lack of cell division, larger cell size, and expressions of senescence-associated p-Galactosidase (SA-β-Gal), p16 and p21 (FIG. 2A-FIG. 2B). FIG. 2A-FIG. 2B confirm senescence of irradiated red tdTOMp16+ cells. FIG. 2A shows that sorted red tdTOMp16+ did not divide (0/960) when cultured as single cells for 2-weeks, while irradiated non-senescent (50/800) and non-irradiated (137/560) cells divided. FIG. 2B shows that senescent markers p16 and p21 (not shown), and SA-D-Gal were significantly upregulated in senescent cells (**, p=0.01; ***, p=0.001; ***, 0.0001; One-way ANOVA and t-test).

RNA-seq was performed in triplicates after extracting the total RNA from: 1) control non-irradiated cells, 2) sorted irradiated tdTOMp16+ red; and 3) irradiated, but tdTOMp16-non-red cells. After RNA-seq, the data was analyzed for principal component analysis (PCA) (FIG. 3) and showed separation of the 3 cell populations and that the three biological repeats were clustered together. Comparing 5 Gy irradiated senescent red cells with 5 Gy non-red non-senescent cells, it was found that 1174 genes were differentially expressed of which 782 were upregulated and 392 were downregulated. In hierarchic clustering of differentially expressed genes by heatmap analysis, the three groups showed profoundly up- and down-regulated genes in red senescent cells (FIG. 4). A customized search for the gene enrichment analysis showed upregulation in senescent cells of three gene categories: including those associated with chemotaxis, phagocytosis, fibrosis.

The ranking of the top 10 genes were analyzed according to their experimental fold change (Table 1). The upregulated genes in irradiated red senescent cells were compared to irradiated non-red cells and a top upregulated gene tyrosine-kinase, Fgr, was discovered, the expression of which was 72-fold elevated. The elevation of Fgr was of greater magnitude than the level observed with any other chemotaxis or phagocytosis related transcript or any downregulated gene (Table 1). The elevated expression of Fgr was confirmed by RT-qPCR (FIG. 5A-FIG. 5C).

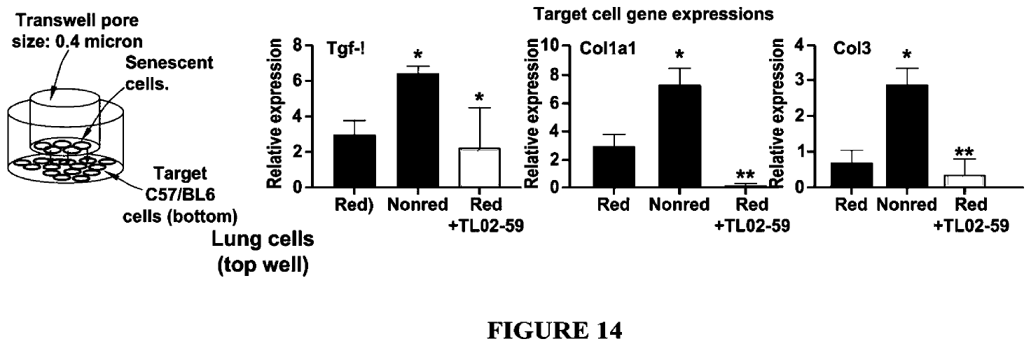

To establish that irradiated tdTOMp16+ senescent cells induce profibrotic genes in other cells, a C57BL/6 target cell line and a noncontact transwell co-culture system were used (Ejaz A et al. Stem Cells. 2019, 37, 791-802) (FIG. 14). Using the tdTOMp16+ stromal cell line, significant induction by RIS red cells of collagen 1al, collagen 3, and TGF-β in C57/B6 target cells was observed. The induction was abrogated by the Fgr inhibitor TL02-59 (Mukherjee A et al. Cell Death Discovery, 2021, 7, 349). It was confirmed that the findings in stromal cells also occur in lung. Red and non-red cells from 20 Gy irradiated tdTOMp16 mouse lungs were tested. Addition of the small molecule specific inhibitor of Fgr TL02-59 also reduced the induction of collagen 3 by RIS red lung cells (FIG. 14).

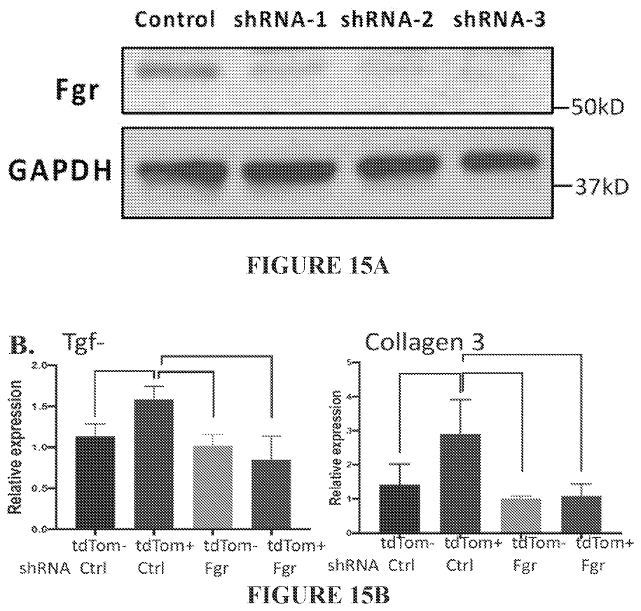

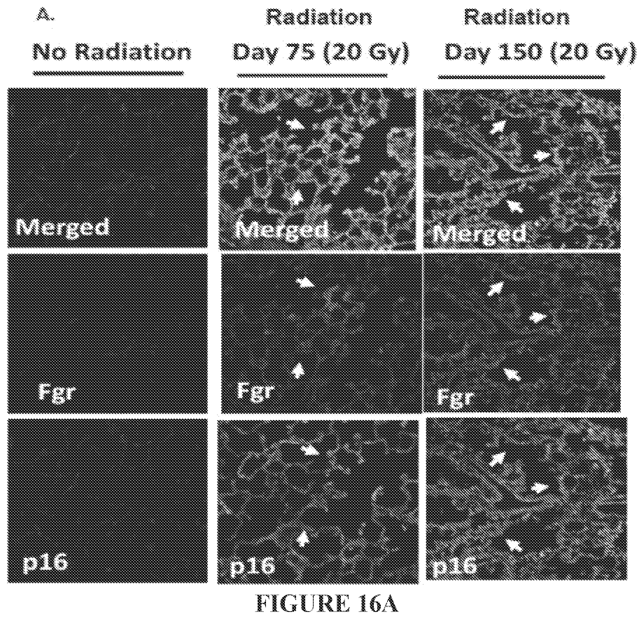

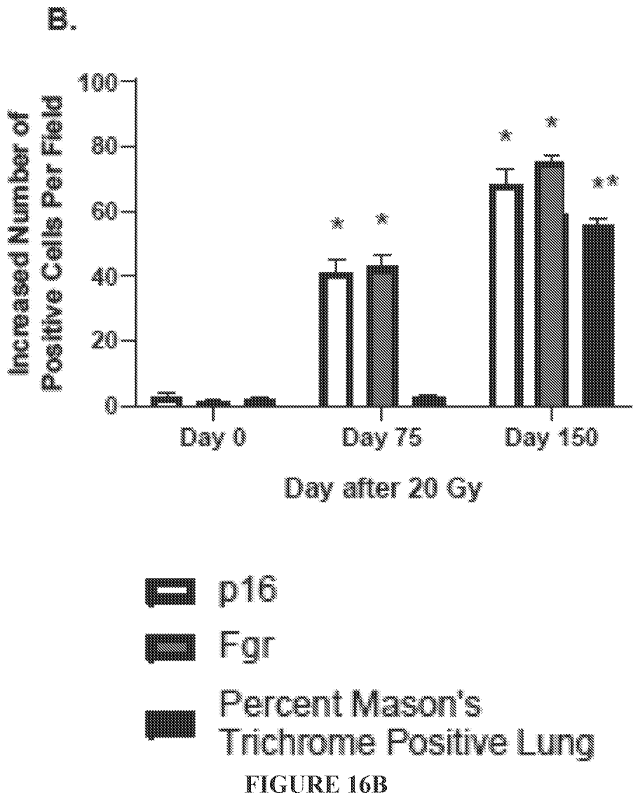

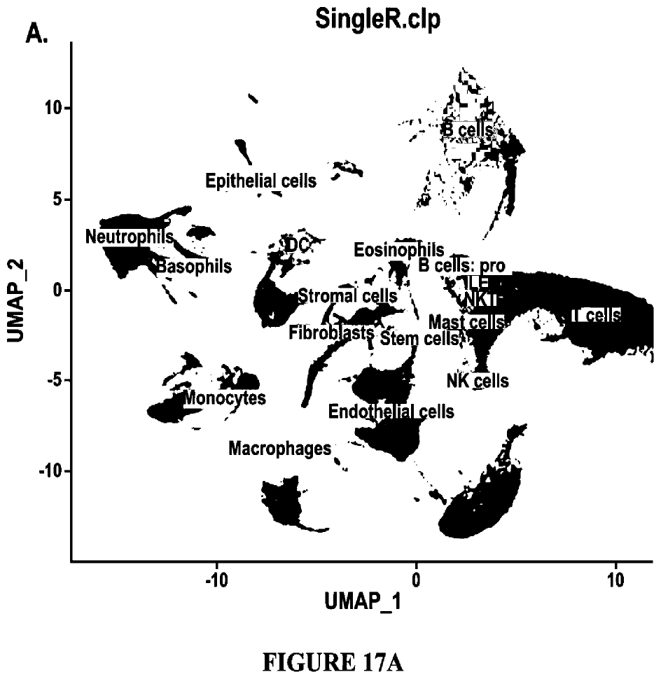

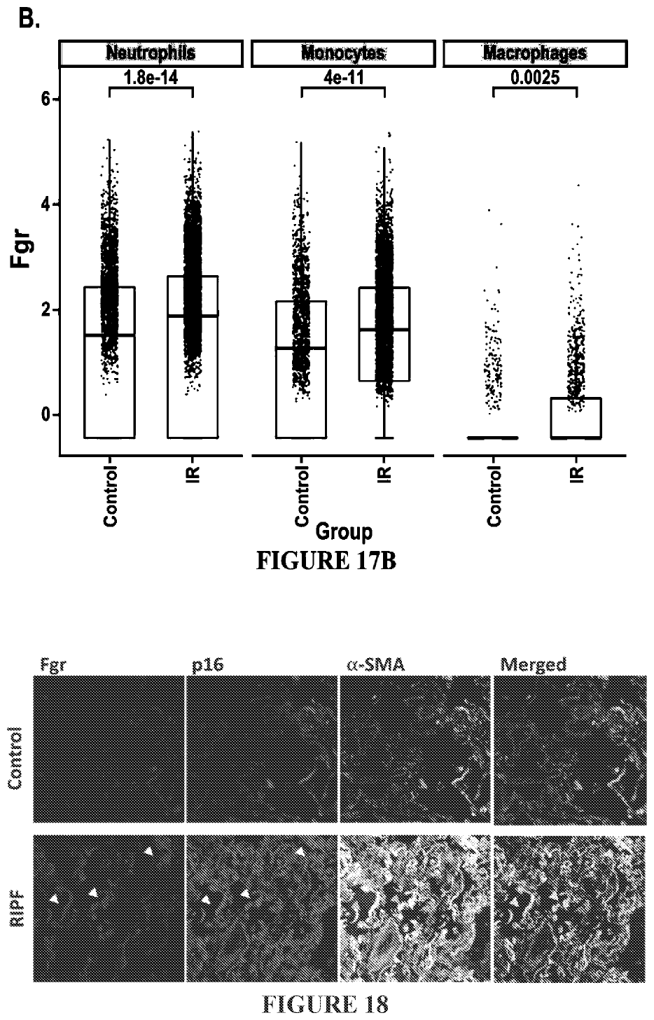

Fgr was successfully knocked down by shRNA in tdTOMp16+ cells in vitro (Mukherjee A et al. Cell Death Discovery, 2021, 7, 349) which reduced the fibrotic gene expression that was induced by control RIS cells (FIG. 15A-FIG. 15B). Data show Fgr+ senescent cells in the irradiated lung prior to pulmonary fibrosis (FIG. 16A-FIG. 16B). By scRNAseq, it was shown that Fgr is induced in RIPF lung immune cells (FIG. 17A-FIG. 17B). Finally, it was shown that Fgr is induced in human RIPF lung tissue (FIG. 18).

### Experimental

Specific Aim 1: Elucidate the mechanism by which downregulation of Fgr in RIS cells reduces their capacity to induce biomarkers of radiation fibrosis.

Sub-Aim IA: Establish that shRNA inhibition of Fgr in RIS cells in vitro or cells isolated from tdTOMp16+ Fgr−/− mouse lungs abrogates their induction of biomarkers of fibrosis. The experiments can use either Fgr shRNA on RIS cells or isolate RIS cells from WT-tdTOMp16+ and Fgr−/− tdTOMp16+ mouse lungs at 150 days after 20 Gy irradiation. For this, Fgr−/−×tdTOMp16+ mice can be generated. In a non-contact transwell coculture system, RIS cells can be plated on the top of the transwell, and the target C57BL/6 mouse tail fibroblast cell line can be plated at the bottom. It can be established that neither shRNA knockdown in RIS cells nor RIS Fgr−/− cells induce fibrosis markers in the target cells. With the tdTOMp16+ RIPF lung cells the effects of Fgr deletion can be compared using: 1) irradiated and sorted red tdTOMp16+ cells. 2) irradiated sorted non-red tdTOMp16+ cells, and non-irradiated tdTOMp16+ cells by both (a) immunostaining for profibrotic genes in target cells including collagen 1-1V and smooth muscle actin and (b) real time qPCR for fibrosis related genes TGF-β1, CTGF, TNF, NF-κB, IL-1, and Col1-Col6 (Ejaz A et al. Stem Cells, 2019, 37, 791-802), and (c) other inflammatory and stress response proteins by Luminex assay (Thermozier S et al. Radiat Res. 2020, 193, 435-450). These experiments can use 12 male and 12 female mice=total 24 mice.

Sub-Aim 1B: Identify the SASP factor(s) released from RIS cells that mediate the induction of profibrotic biomarkers in target cells in transwells. The SASP factors secreted by the tdTOMp16+ RIS cells expressing either control or Fgr-shRNA can be analyzed by SASP cytokine array (Maciel-Baron L A et al. Age (Dordr), 2016, 38, 26). Sixty-two cytokines including the SASP proteins identified in RNA-seq can be analyzed following manufacturer's protocol using kit Mouse Cytokine Array C3 (RayBiotech Inc., Norcross, GA). Briefly, the sample bound membrane can be incubated with biotinylated antibody cocktail and then with HRP-Streptavidin. Chemiluminescence imaging system can be used to analyze the data. Lung cells can be analyzed at days 50, 75, 120 and 150 post irradiation (20 Gy) from both wt and Fgr−/− mice and compared by SASP cytokine array. The SASP cytokine array can be performed on both intracellular proteins of the lung tissue lysate and extracellular proteins from the media after culturing the freshly isolated cells for 48-72 hours. The isolated tdTOMp16+ mouse lung cells in culture can be treated with TL02-59 and cytokine array can be performed and compared with RIS Fgr−/− cells. These experiments can use 5 mice of 5 time points for both wt and Fgr−/− mice=total 50 mice.

Physics: Cells can be irradiated in vitro using a Cesium-137 JL Shepherd Model 68 irradiator at 3 Gy per minute.

Statistics: Two-way ANOVA models can be used where treatment and time and their interaction are factors followed by Tukey's multiple comparison tests.

Results and Interpretation: Fgr is a member of the tyrosine kinase family (Beyer C et al. Biochim Biophys Acta. 2013, 1832(7), 897-904; Grimminger F et al. Eur Respir J, 2015, 45(5), 1426-1433; Kovacs M et al. J Exp Med, 2014, 211(10), 1993-2011; Li H et al. Biomed Pharmacother. 2020, 127, 110183) and its potential profibrotic functions have been recently described (Medina I et al. Circulation, 2015. 132(6), 490-501). Senescent cells isolated from Fgr−/− tdTOMp16+ mice may not induce collagen 1A, collagen 3, and TGF-μ in target cells in transwells. shRNA mediated Fgr knockdown in RIS cell line and RIS lung cells, which are FACS purified from irradiated wt and Fgr−/− tdTOMp16+ mice, may not produce secreted profibrotic proteins, and may not induce profibrotic genes in target cells. Several components of the SASP, which are pro-fibrotic, may be identified. Experiments with Fgr inhibitor TL02-59 can confirm the data observed by Fgr shRNA and in Fgr −/− cells.

It is possible that Fgr deletion in cells can cause a compensatory induction of related tyrosine kinases like Hck and Lyn. This adaptive response can be examined by western blotting using phospho antibodies and the fibrotic response can be overcome with triple knockout mice that have been obtained.

TL02-59 is Fgr specific in the nanomolar concentration (Weir M C et al. ACS Chem Biol 2018, 13, 1551-1559). This result can be confirmed by assessing phosphorylation of other kinases. shRNA knockdown of other kinases in senescent cells can be used to determine what transcripts and secreted proteins are absent compared to controls. Primary human lung fibroblasts (ATCC) can be used to confirm results from mouse stromal tdTOMp16 cells. The mediator(s) released by RIS cells that induce profibrotic genes can be identified by cytokine array. This approach can potentially miss a mediator. Analysis of all secretory proteins by labelling, followed by mass-spectrometry, can be performed. It can also be test if published mitigator MMS350 of fibrosis (Kalash R et al. Radiat Res. 2013, 180(5), 474-490) inhibits Fgr. If the Fgr stimulated SASP in RIS cells does not reveal a protein mediator, oxidized phospholipids, microRNA, or other mediators that are absent after Fgr shRNA knockdown and in Fgr−/− RIS cells can be identified.

Specific Aim 2: Determine the phenotype(s) of Fgr positive RIS cells, which appear in irradiated lungs prior to the evolution of fibrosis.

Sub-Aim 2A: Determine the lung cell types that express increased Fgr and senescent biomarkers prior to pulmonary fibrosis by single-cell RNA-seq. Lung single cell suspension can be prepared from 20 Gy thoracic irradiated and control C57BL/6 mice at 0, 75, and 150 days. It takes 150-180 days for mice to develop fibrosis after 20 Gy thoracic radiation. It was shown that Fgr and senescent markers are induced as early as day 50 (FIG. 16A-FIG. 16B) (Mukherjee A et al. Cell Death Discovery, 2021, 7, 349). For scRNAseq, Chromium Next GEM Single Cell 3′ Reagent Kits v3.1 can be used (10X Genomics, Pleasanton, CA) following manufacturer's protocol, the quality of raw reads can be assessed using FastQC, and Cell Ranger maps reads against mouse genome GRCm38 can be generated using STAR aligner, followed by Unique Molecular Identifier (UMI). Cell type assignment can be performed using SingleR (36) (v1.2.4) with the mouse Immunological Genome Project (ImmGen) as the reference database. The scRNAseq on different time points can reveal the dynamic nature of senescence that occurs prior to radiation-induced pulmonary fibrosis. To identify the senescent cells, a panel of senescence genes expressed in different cell types can be used and a senescent score can be assigned. These experiments can use 4 mice per time point for a total of 12 male and 12 female mice=total 24 mice.

Sub-Aim 2B: Establish the phenotype(s) of irradiated lung cells that produce elevated levels of Fgr after irradiation: (endothelial vs. epithelial vs. monocytes, macrophage). First, Fgr expressing cell types from scRNAseq can be validated in control C57BL/6 mice by qPCR after FACS sorting of endothelial (CD45−, CD31+), epithelial (CD45−, EPCAM+), monocytes, macrophages, neutrophils, and dendritic cells (using appropriate combinations of CD45+,F4/80+, CD24+/−, CD11c+,CD11b+/− markers) (Misharin A V et al. Am J Respir Cell Mol Biol 2013, 49, 503-510). To confirm the induction of Fgr in certain cell types, as they become senescent after thoracic irradiation, tdTOMp16+ mice can be used. From the irradiated mouse lungs at 0, 75, 100, 150 and 180 days, tdTOM+ and tdTOM− cells can be sorted first and then epithelial, endothelial, monocytes, neutrophils, and dendritic cells can be isolated and qPCR can be performed for validation. Number of mice at specific times for these experiments can be 5-timepoints×5 mice×3 replicates, 75 males and 75 female mice=150 total mice. Transwell experiments can be performed using isolated tdTOMp16+ RIS cells to assess the primary cell type responsible of inducing fibrotic genes in target cells.

Physics: Cells can be irradiated in vitro using a Cesium-137 JL Shepherd Model 68 irradiator at 3 Gy per minute. Mice can be thoracic irradiated (Kalash R et al. Radiat Res. 2013, 180(5), 474-490) using 6 MV photons delivered at 600 monitor units per minute by a Varian True Beam Irradiator. The irradiation field can be 2 cm×40 cm with an SSD of 100 cm.

Statistics: Two-way ANOVA models can be used where treatment and time and their interaction are factors followed by Tukey's multiple comparison tests. For fibrosis studies, a sample size of 7 per group can provide 83% power to detect the expected difference of 1.7 effect size at 0.05 significance level using the two-sided two-sample t-test or Wilcoxon rank sum test where appropriate.

Sub-Aim 2C: Determine the levels of Fgr expression and senescence in the human RIPF lung tissue. Immunohistochemistry can be performed in serial sections using cell specific markers as described in Aim 2B and expression of Fgr and senescent biomarkers can be evaluated in human RIPF samples from radiotherapy patients who require surgery months to years after thoracic irradiation. To confirm the induction of Fgr, qPCR and Westem blotting can be performed. The activation status of Fgr can also be confirmed by western blot using human phospho-Fgr antibody. Human tissue experiments will be carried out following the regulations of the University of Pittsburgh (approved protocol STUDY21080019).

Results and Interpretation: Senescent epithelial phenotype can appear in the lung as early as 50-75 days after 20 Gy thoracic irradiation and produce Fgr. Macrophage/monocyte cells that are known to have baseline Fgr expression can have Fgr upregulation at later time points, at 75-150 days after IR. The dynamics of Fgr expression in senescent cells along the course of radiation-induced pulmonary fibrosis can allow for the determination of when a therapeutic intervention could be applied. The identification of Fgr expressing senescent cell-types can allow a study of cell-type specific conditional Fgr knockout mice.

Senescent cells that were sorted from irradiated tdTOMp16+ mouse lung cells may not have the same Fgr level, as that measured by RNA-seq, as that of a bone marrow stromal cell line. It has been confirmed that of Fgr induction in mouse RIPF and human RIPF. Detailed analysis of other profibrotic genes in the senescent lung cells may be needed. Cytokines secreted from lung cell (SASP factors) may recruit monocyte/macrophages of bone marrow origin (Mukherjee A et al. In Vivo 2021. 35, 3053-3066). It's been shown that CD 11b+, non-resident macrophages and Ly-6C^{hi} monocytes are determinants of pulmonary fibrosis induced by selective type II alveolar epithelial cell injury (Osterholzer J J et al. J Immunol 2013, 190, 3447-3457). These cell types can be analyzed by FACS.

Specific Aim 3: Establish that blocking Fgr in vivo reduces pulmonary fibrosis.

Sub-Aim 3A: Quantitate the reduction of fibrosis in the lungs of TL02-59 treated tdTOMp16+ mice after 20 Gy thoracic irradiations. At day 75 after 20 Gy thoracic irradiations these mice display RIS in lungs with no detectable fibrosis and there is fibrosis at day 150 (FIG. 16A-FIG. 16B). The Fgr inhibitor TL02-59 can be given 3 days a week by gavage starting at day 75 for 10 weeks at a daily dose of either 1 or 10 mg/kg in 10% DMSO plus 90% corn oil. Control mice receive vehicle only. Pharmacokinetics of TL02-59 in mice demonstrated that TL02-59 is orally bioavailable and displays a plasma half-life of 6 h in mice, making it an attractive candidate mitigator for in vivo evaluation. After 10 weeks of treatment, at day 150 post radiation the mice are sacrificed, and lung tissue collected. Gene expression analysis can be performed by qPCR (Collagens, MMPs, α-SMA, NF-kB), biochemical analysis can be performed by measuring hydroxyproline content, and histological analyses (Masson's trichrome and H&E staining) of radiation-induced pulmonary fibrosis can be formed using published methods (Kalash R et al. Radiat Res. 2013, 180(5), 474-490; Epperly M W et al. Radiat Res 2021, 196, 235-249). A modified Ashcroft scale can be used for quantification of lung fibrosis in mice (Hubner R H et al. Biotechniques 2008, 44, 507-514). All animal studies can be carried out in male and female mice and conducted in accordance with the guidelines and regulations established by the University of Pittsburgh Institutional Animal Use and Care Committee (approved protocol 18022000). To perform histology, qPCR, and cell sorting experiments, for 3 TL02-59 doses (vehicle, 1 mg/kg, and 10 mg/kg), these experiments can use 10×3 male and 10×3 female mice. Total mice=60

Sub-Aim 3D: Determine the level of Fgr in lung cell phenotypes after 20 Gr irradiation and TL02-59 treatment. Over a period of 150 days, expression of Fgr in senescent lung cell types (identified in Aim 2) after TL02-59 treatment can be measured. The magnitude of senescence can be measured by immunohistochemistry in serial sections using senescence markers p16 and SA-β-gal.

Sub-Aim 3C: Establish reduction of fibrosis in the lungs of 20 Gy irradiated C57BL/6 Fgr−/− mice. At day 150 post 20 Gy thoracic irradiation, Fgr−/− mice can be sacrificed, and lung tissue excised. Histological scoring can be performed using a modified Ashcroft scale (Hubner R H et al. Biotechniques 2008, 44, 507-514), gene expression analysis can be performed by qPCR, biochemical analysis can be performed by measuring hydroxyproline content, and histological analysis (Masson's trichrome and H&E staining, immunostaining by collagens, and α-SMA) of radiation-induced pulmonary fibrosis can be performed using published methods (Kalash R et al. Radiat Res. 2013, 180(5), 474-490; Epperly M W et al. Radiat Res 2021, 196, 235-249). These experiments can use a total of the control 2×7=14 male and 2×7=14 female mice=total 28 mice. To compare these results with control mice, the mice from the control vehicle group of Aim 3B can be used.

Physics: Mice can be thoracic irradiated (Kalash R et al. Radiat Res, 2013, 180(5), 474-490) using 6 MV photons delivered at 600 monitor units per minute by a Varian True Beam Irradiator. The irradiation field can be 2 cm×40 cm with an SSD of 100 cm.

Statistics: Two-way ANOVA models can be used where treatment TD02-59 vehicle or radiation dose are factors followed by Tukey's multiple comparison tests. For fibrosis studies, a sample size of 7 per group can provide 83% power to detect the expected difference of 1.7 effect size at 0.05 significance level using the two-sided two-sample t-test or Wilcoxon rank sum test where appropriate.

Results and Interpretation: TL02-59 treatment specifically inhibits Fgr with partial inhibition observed at 0.1-1 nM and complete inhibition above 10 nM. In contrast, other similar kinases require a 10-to-100-fold greater dose for inhibition (100-1000 nM). In a 10 nM concentration, TL02-59 inhibits only Fgr. TL02-59 given daily from day 75 after 20 Gy thoracic irradiation can reduce fibrosis. Fgr−/− mice can be resistant to fibrosis like the TL02-59 treatment group.

If no reduction of radiation-induced pulmonary fibrosis after TL02-59 administration starting at day 75 is observed, the experiments can be repeated beginning delivery of TL02-59 on day 50 or earlier. Weekly or monthly administration of TL02-59 can also be compared and Fgr positive cells and fibrosis can be measured at day 150 after 20 Gy thoracic irradiation to examine the effect on the progression of fibrosis. If Fgr inhibitor TL02-59 or lack of Fgr in Fgr−/− mice does not reduce production of Fgr by RIPF-induced senescent cells in the lungs in vivo, a lung cell type specific Fgr conditional knockout mouse can be derived (Rhieu B H et al. In Vivo. 2014, 28. 1033-1044).

### Example 5—Ionizing Irradiation-Induced Fgr in Senescent Cell Mediates Fibrosis

Abstract: The role of cellular senescence in radiation-induced pulmonary fibrosis (RIPF) and the underlying mechanisms are unknown. Radiation-induced senescent tdTOMp16 positive mesenchymal stem cells were isolated, their absence of cell division was established, then levels of irradiation-induced expression of biomarkers of senescence was measured by RNA-seq analysis. A Log 2 6.17-fold upregulation of tyrosine kinase Fgr was identified, which was a potent inducer of biomarkers of fibrosis in target cells in non-contact co-cultures. Inhibition of Fgr by shRNA knockdown did not block radiation-induced senescence in vitro; however, both shRNA knockdown, or addition of a specific small-molecule inhibitor of Fgr, TL02-59, abrogated senescent cell induction of profibrotic genes in transwell-separated target cells. Single-cell RNA-seq (scRNAseq) analysis of mouse lungs at day 150 after 20 Gy thoracic irradiation revealed upregulation of Fgr in senescent neutrophils, and macrophages before detection of lung fibrosis. Thus, upregulated Fgr in radiation-induced senescent cells mediates radiation-induced pulmonary fibrosis and is a potential therapeutic target for the prevention of this radiation late effect.

Introduction: The histopathology of radiation pulmonary fibrosis (RIPF) is similar to that of idiopathic pulmonary fibrosis (IPF), silicosis, and in post-infection conditions (Martinez F J et al. Nat Rev Dis. Prim. 2017, 3, 17074; Jarzebska N et al. Front Med. 2020, 7, 585756; Li N et al. Environ Toxicol. 2021. https://doi.org/10.1002/tox.23124; Zou J N et al. PLoS ONE. 2021, 16, e0248957). Recent reports indicate cellular senescence precedes both idiopathic and radiation-induced pulmonary fibrosis (RIPF) (He Y et al. Transl Res. 2019, 209, 14-21; 6. Epperly M W et al. Radiat Res. 2021. https://doi.org/10.1667/RADE-20-00286.1; Su L et al. Cell Death Dis. 2021, 12, 527). Ionizing irradiation induces senescent cells (Epperly M W et al. Radiat Res. 2021. https://doi.org/10.1667/RADE-20-00286.1; Sivananthan A et al. Radiat Res. 2019, 191, 139-53), which display specific physiological properties including cell cycle exit, senescent-associated β-galactosidase (SA-β-GAL) expression, and secretion of senescence-associated secretory phenotype (SASP) proteins (Mora A L et al. Nat Rev Drug Discov. 2017, 16, 755-72; Herranz N et al. J Clin Investig. 2018, 128, 1238-46; Althubiti M et al. Cell Death Dis. 2014, 5, e1528; Okuda R et al. J Thorac Dis. 2019, 11, 857-64). Cell cycle arrest in senescent cells is associated with an increase in cyclin-dependent kinase inhibitors p21, and p16 which are components of tumor-suppressor pathways regulated by p53 and the retinoblastoma (pRB) proteins respectively (Althubiti M et al. Cell Death Dis. 2014, 5, e1528). SASP contains pro-inflammatory cytokines, matrix-remodeling enzymes, and extracellular vesicles (Herranz N et al. J Clin Investig. 2018, 128, 1238-46; Althubiti M et al. Cell Death Dis. 2014, 5, el 528; Okuda R et al. J Thorac Dis. 2019, 11, 857-64). SASP proteins also include chemotactic proteins like Ccl2, Ccl4, and transforming growth factor beta (TGFβ), which is a profibrotic cytokine (Okuda R et al. J Thorac Dis. 2019, 11, 857-64). The SASPs in radiation-induced senescent (RIS) cells (Chen Z et al. Oncol Rep. 2019, 42, 883-94) differ from oncogenes (Cisowski J et al. Oncogene. 2016, 35, 1328-33), chemotherapeutic drugs (Saleh T et al. Cancers. 2020, 12, 822), or telomere shortening (Cisowski J et al. Oncogene. 2016, 35, 1328-33; Hernandez-Segura A et al. Curr Biol. 2017, 27, 2652-60) induced senescence.

Prior studies with mixed senescent and non-senescent cells have limited strong conclusions about their specific properties (Hernandez-Segura A et al. Curr Biol. 2017, 27, 2652-60). A method by which to purify senescence cells would require the elimination of non-senescent cells in the targeted population (Itahana K et al. Methods Mol Biol. 2007, 371, 21-31). A mouse strain was used, in which senescent cells can be sorted by td-Tomato fluorochrome, linked to induction of senescence biomarker p16 (Sessions G A et al. FASEB J. 2019. 33, 12364-73). A cell line derived from tdTOMp16+ mouse bone marrow was used to sort a pure population of RIS cells, which display a unique gene expression profile compared to irradiated non-senescent cells in the same experiments and in non-irradiated control cells. Using RNA-seq analyses, it was determined that a specific tyrosine kinase, Fgr, is upregulated in senescent cells. Senescent cell induction of fibrosis biomarkers was blocked by the addition of an inhibitor of Fgr, TL02-59 (Weir M C et al. ACS Chem Biol. 2018, 13, 1551-9), or by shRNA knockdown of Fgr prior to irradiation. Single-cell RNA-seq (ScRNAseq) analysis of irradiated mouse lungs revealed Fgr upregulation in monocytes, neutrophils, and macrophages at 150 days. Therefore, Fgr mediates senescent cell induction of fibrosis, and its inhibition by TL02-59 provides a strategy by which to ameliorate radiation-induced pulmonary fibrosis.

### Results

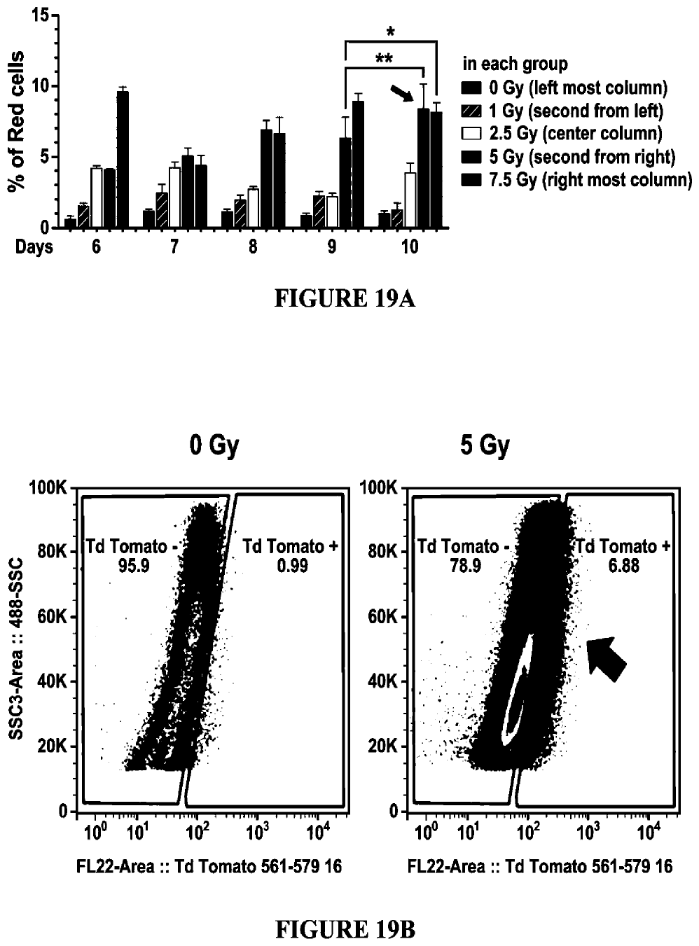

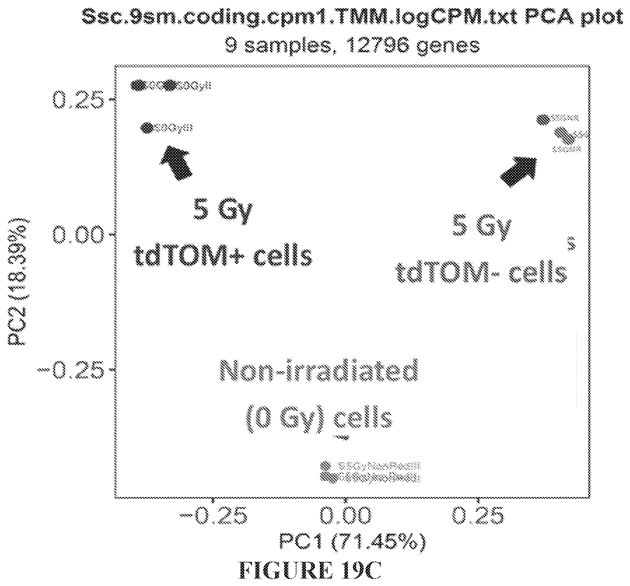

Radiation induction and sorting of senescent tdTOMp16+ cells in vitro. A mesenchymal stem cell (MSC) line derived from tdTOMp16+ mouse long-term bone marrow cultures (Sivananthan A et al. Radiat Res. 2019, 191, 139-53) was irradiated to increasing doses at either 50% or 100% cell density. After 10 days, 7-10% of cells at 50% cell density during irradiation, were tdTOM+ (red) with both 5 Gy and 7.5 Gy groups (FIG. 19A). Since 7.5 Gy increased cell death, 5 Gy was used for sorting experiments. By FACS, 7-10% tdTOM+ cells were isolated (FIG. 19B). RNA-seq analysis was performed using three independent samples for every three groups: irradiated senescent (tdTOM+) cells, irradiated nonsenescent (tdTOM−) cells, and non-irradiated cells. Large-scale sorting was necessary since 9% of the total irradiated cells were senescent (tdTOM+) and 90% were non-senescent (tdTOM−).

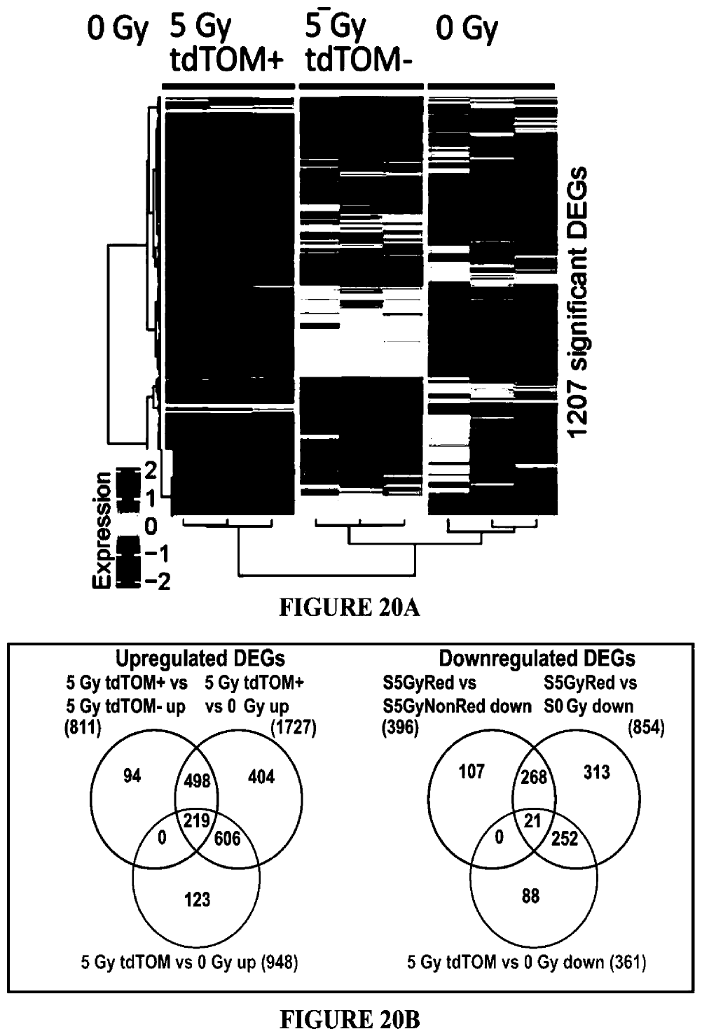

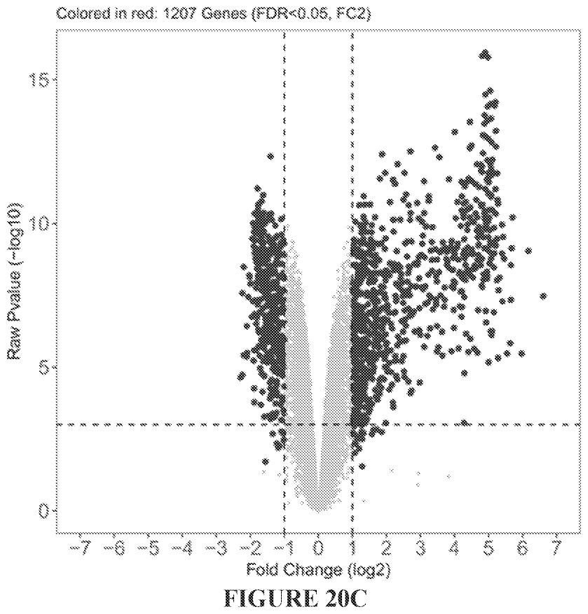

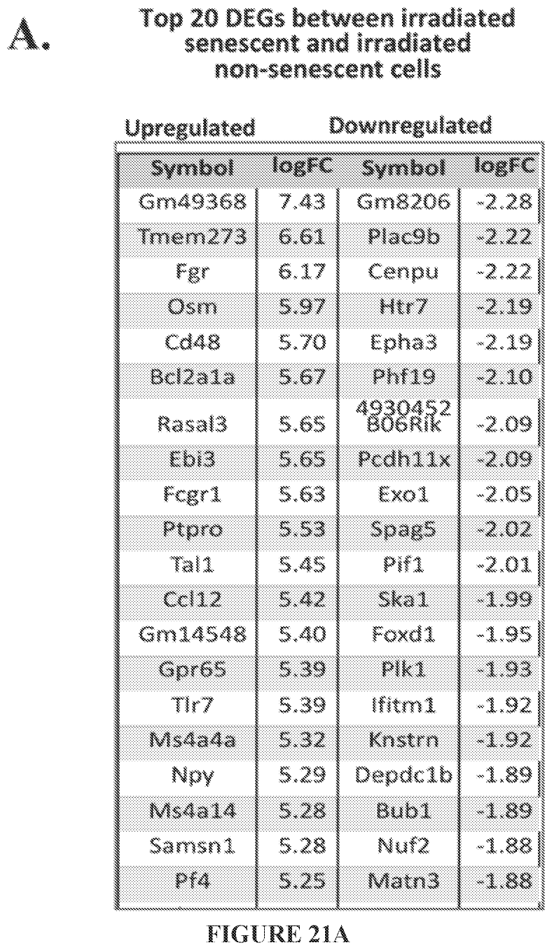

Principal component analysis (PCA) was performed on all genes (12796) present in the RNA-seq dataset and showed the variance of gene expression within and between sample groups. The PCA distribution showed distinct separation of the three groups and all three replicates were clustered together (FIG. 19C). A total of 1207 differentially expressed genes (DEGs) were statistically significant (log 2 fold change ≥2 and an adjusted P value≤0.05) between the irradiated tdTOM+ senescent, irradiated, tdTOM− non-senescent, and non-irradiated cells shown in FIG. 20A-FIG. 20C.

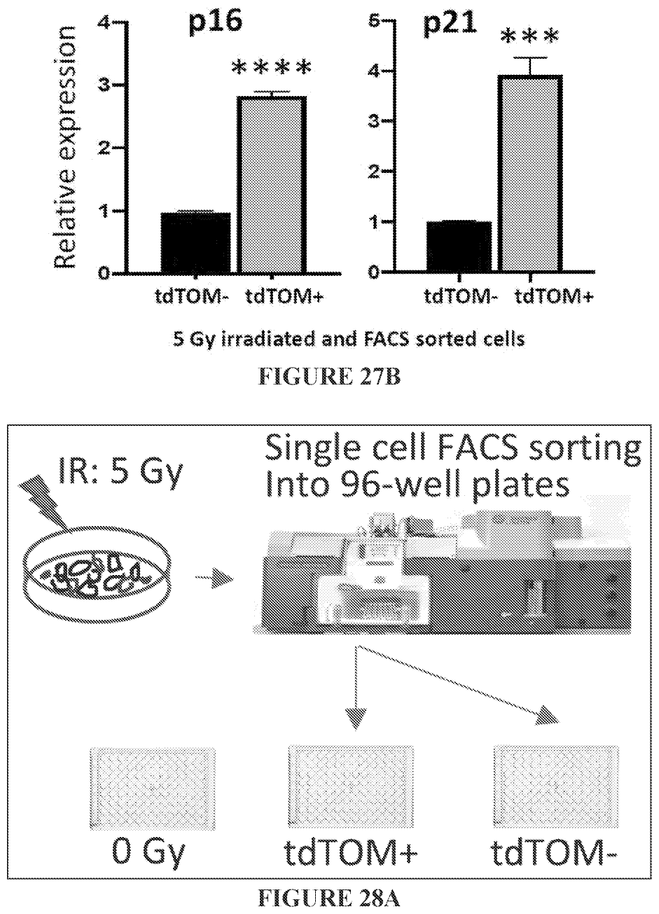

Radiation-induced tdTOMp16+ senescent cells are non-dividing and are morphologically and biochemically distinct. Since p16 expression is transiently upregulated by DNA damage (Mirzayans R et al. Biochem Res Int. 2012, 2012, 951574), and one p16 allele is uninterrupted in tdTOMp16+ cells (Sessions G A et al. FASEB J. 2019, 33, 123(4-73), it was determined whether the p16 allele and other biomarkers of senescence were induced by irradiation including SA-β-Gal and p21. A similar percentage of tdTOM+ cells were observed to also be SA-β-Gal-positive. Furthermore, p16 and p21 were significantly upregulated in tdTOM+ senescent cells compared to irradiated tdTOM− non-senescent cells (FIG. 27A, FIG. 27B).

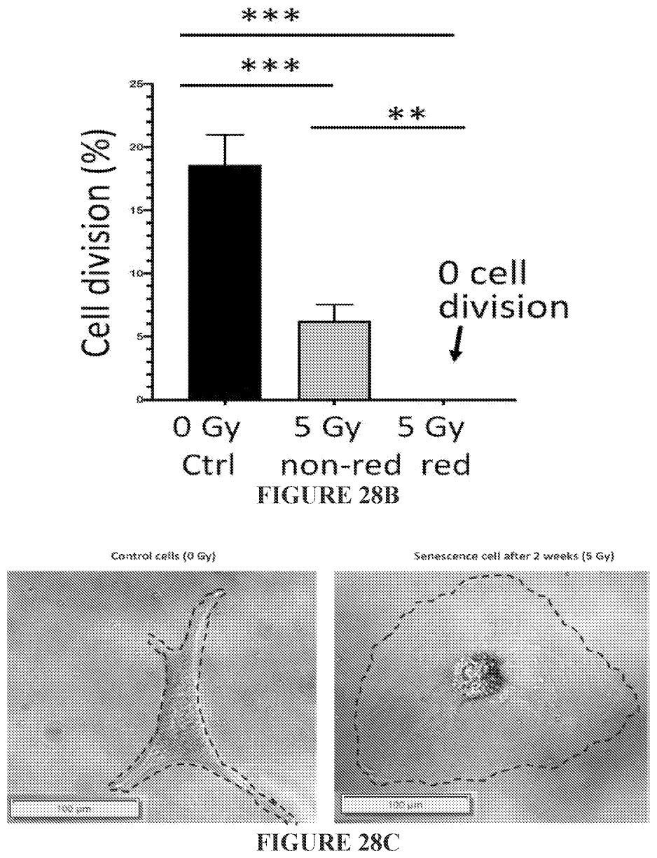

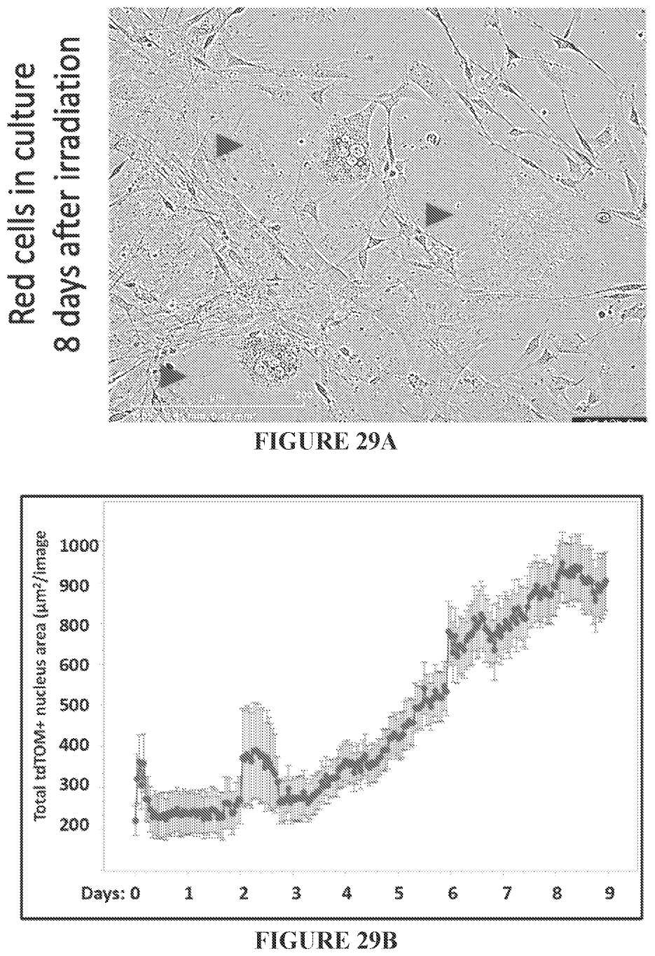

Senescent cells as single cells in 96-well plates were next followed over 4 weeks for cell division. None of the 960 senescent (red) tdTOM+ cells divided after 2 weeks and 137 remained intact. In contrast, 50 of 800 irradiated, non-senescent (non-red) cells divided. Non-irradiated control cells (0 Gy) revealed cell division by 104 of 560 single cells (FIG. 28A, FIG. 28B). It was observed that senescent tdTOM+ cells were larger and circular after 2 weeks compared to tdTOM− cells, similar to previously published data (Aranda-Anzaldo A. Aging. 2009, 1, 598-607). Rhe motility of 5-Gy irradiated TOMp16+ cells was tracked for 2 weeks and real-time senescence was observed, including the appearance of td-tomato red fluorescence in cells that also became large and circular (FIG. 28C. FIG. 28D, and FIG. 29A). Quantification of red cells showed a steady increase of tdTOM+ cells after radiation that becomes stable at day 9 (FIG. 29B). Thus, tdTOM+ red cells showed no cell division and changed shape, while tdTOM− cells that were also irradiated, but non-senescent did divide.

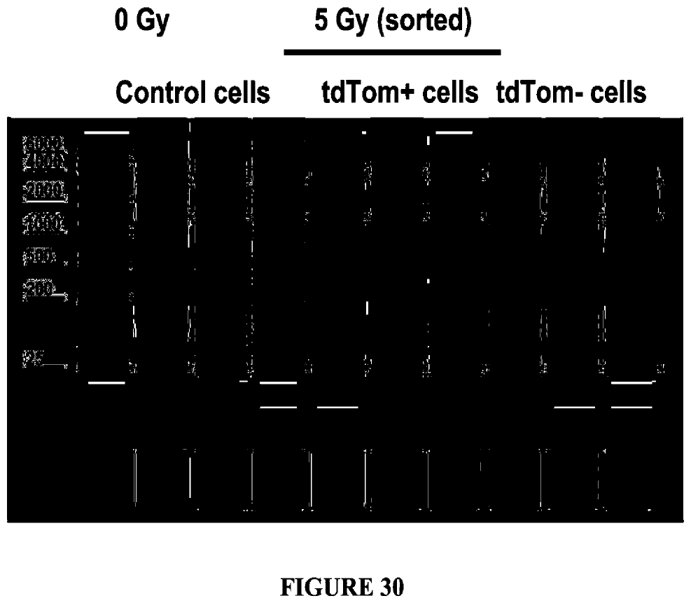

Distinct gene expression profiles of radiation-induced senescent cells by RNA-seq. To determine if tdTOM+ senescent cells showed a unique pattern of up- or down-regulating genes, the RNA-seq data was analyzed as described in FIG. 19C. Cells were sorted into tubes containing tissue culture medium, centrifuged, immediately lysed in Trizol reagent to preserve RNA quality (FIG. 30). The relative expression of differentially expressed genes (DEGs) in tdTOM+ senescent cells was clearly distinct from that of the other two groups (FIG. 20A). Furthermore, the irradiated, but non-senescent cells were different from non-irradiated cells (FIG. 20A, FIG. 20B).

The distribution of the differentially expressed genes (DEGs) between the three cell groups is represented in the Venn diagram (FIG. 20B). Of 1944 upregulated DEGs in senescent cells, there were 1811 upregulated gene transcripts in irradiated tdTOM+ vs. irradiated tdTOM− groups. There were 1727 transcripts detected in both irradiated tdTOM+ and non-irradiated groups. There were 948 upregulated transcripts detected in both irradiated non-senescent and non-irradiated groups (FIG. 20B). The volcano plots show the log 2 (fold change) versus the −log 10 (adjusted P value) for all of the genes detected in the RNA-seq analysis (i.e., for both DEGs and non-DEGs; FIG. 20C). The gray color represents genes with no significantly different expression in senescent in tdTOM+ cells, while the red color indicated overexpression or under expression. The data establish, tdTOM+ senescent cells differ from both irradiated non-senescent and control unirradiated cell groups with respect to gene expression patterns.

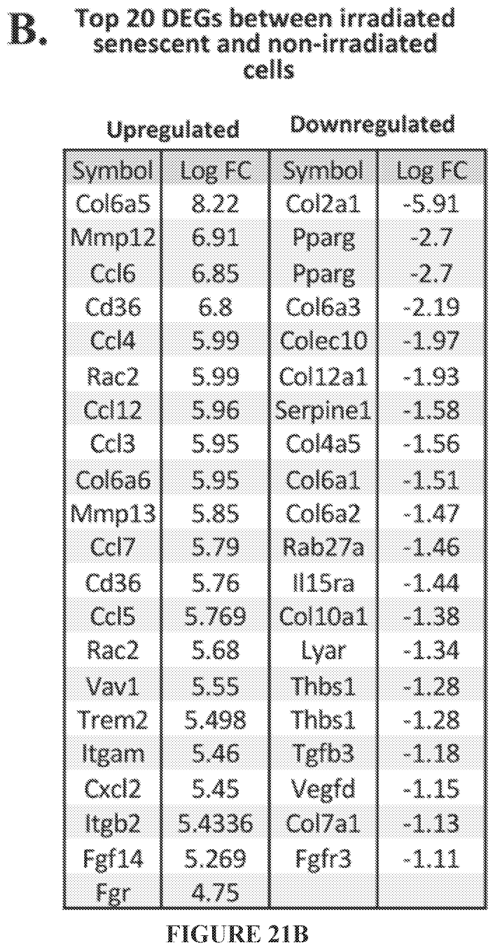

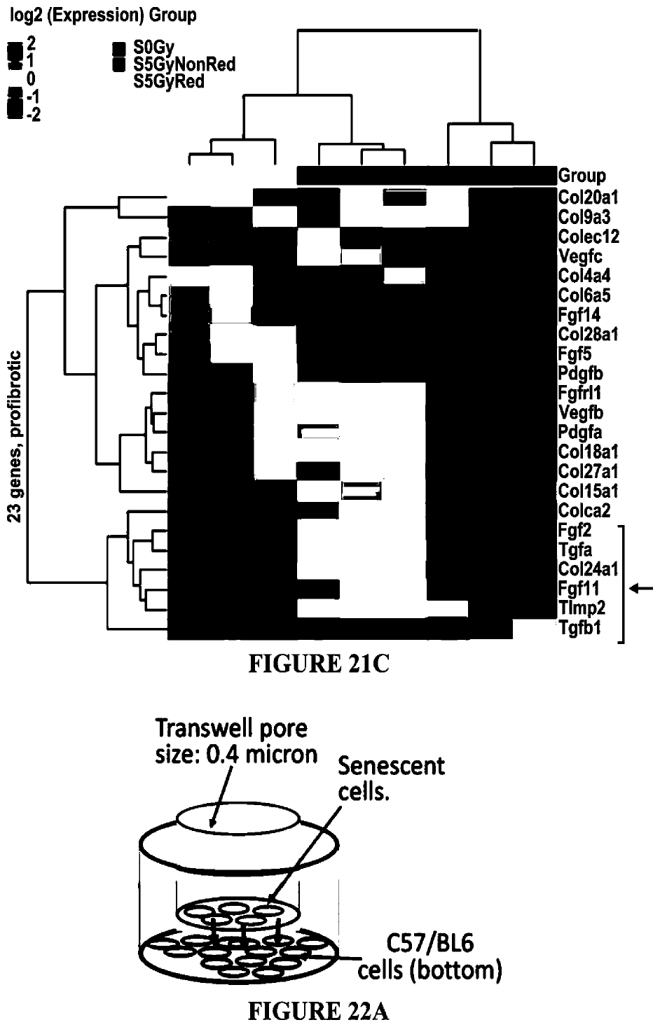

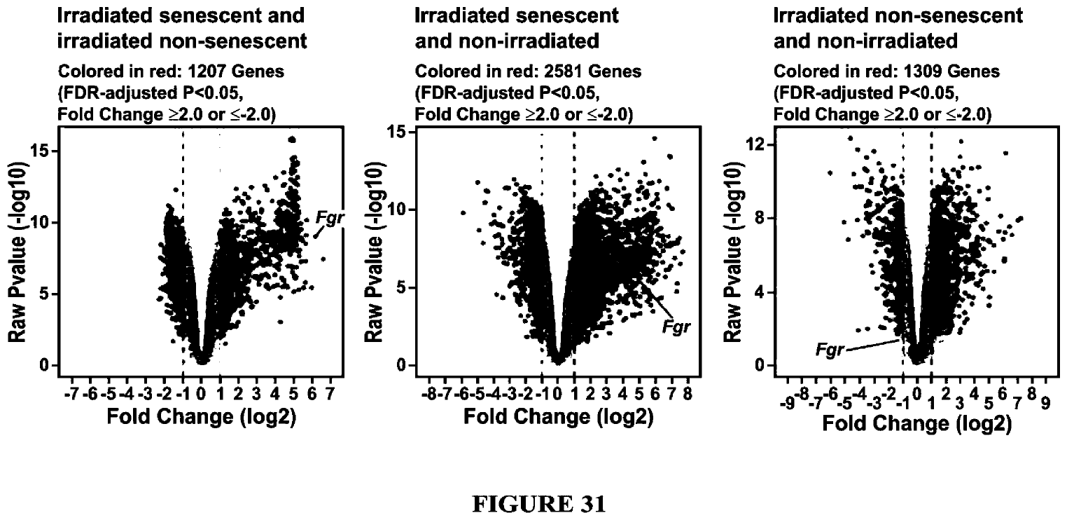

Gene transcripts associated with fibrosis are upregulated in radiation-induced senescent cells. The top 20 overall up- and down-regulated differentially expressed genes (DEGs) in senescent cells (FIG. 21A) compared to the irradiated non-senescent tdTOM− cells and control nonirradiated cells were listed (FIG. 21B). The nonreceptor Src-kinase, Fgr was prominently upregulated (6.17-fold) in senescent cells. Other DEGs in senescent cells included Oncostatin M (Osm), Bcl2A1a and Ccl12 (5.97-fold, 5.67-fold, and 5.42-fold, respectively). Osm is involved in p16- and p53-independent Stat3-mediated activation of senescence (La Belle A A et al. Cell Cycle. 2017, 16, 497-8), Bcl2Ala is a member of Bcl2 family which inhibits apoptosis, suggested as necessary for senescent cells to survive (Metais J Y et al. PLoS ONE. 2012, 7, e48267). The chemokine Ccl12 is known to recruit fibrocytes in the pathophysiology of lung fibrosis (Moore B B et al. Am J Respir Cell Mol Biol. 2006, 35, 175-81). Among the top downregulated genes in senescent cells were the centromere protein U (Cenpu), EphA3, and Foxd1 (−2.22, −2.19, and −1.95-fold, respectively). Cenpu is known to be under-expressed in senescent cells (Zhang Q et al. Eur Rev Med Pharm Sci. 2018, 22, 7768-77), EphA3 has been shown to inhibit senescence (Avelar R A et al. Genome Biol. 2020, 21, 91). Since Foxd1 upregulation by YAP decreases senescence (Kasmann L et al. Radiat Oncol. 2020, 15, 214), downregulation in these senescence cells was expected. FIG. 21B shows the top 20 up and downregulated genes in senescent cells relative to non-irradiated cells. Except for Ccl12, these genes were different from the DEGs found when comparing senescent cells with irradiated non-senescent cells. Fgr was not among those top 20 but was upregulated 4.75-fold. Further investigation of the decline in Fgr induction (−1.42-fold) when comparing irradiated senescent vs non-irradiated cells (4.75-fold) as opposed to irradiated senescent vs irradiated non-senescent cells (6.17-fold) revealed that the decline is slight and likely owing to the baseline tdTOMp16 expression in non-irradiated cells (FIG. 31).

Next, 23 known profibrotic genes were compared between tdTOM+ senescent cells, tdTOM− irradiated, but non-senescent cells, and control non-irradiated cells (FIG. 21C). The heat map shows that profibrotic genes were significantly upregulated in tdTOM+ cells compared to irradiated non-senescent and control non-irradiated cells. Some profibrotic genes were detectably induced in irradiated non-senescent cells relative to control non-irradiated cells. The major targets including Tgfβ1. Tgf-α, Fgf2, Timp2 were induced in tdTOM+ senescent cells (FIG. 21C, arrow), but not in the irradiated but non-senescent cells.

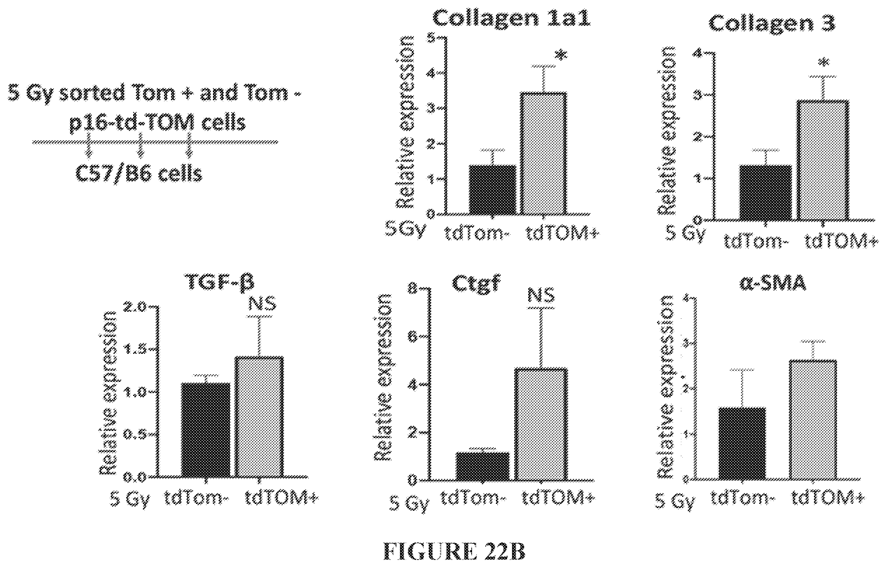

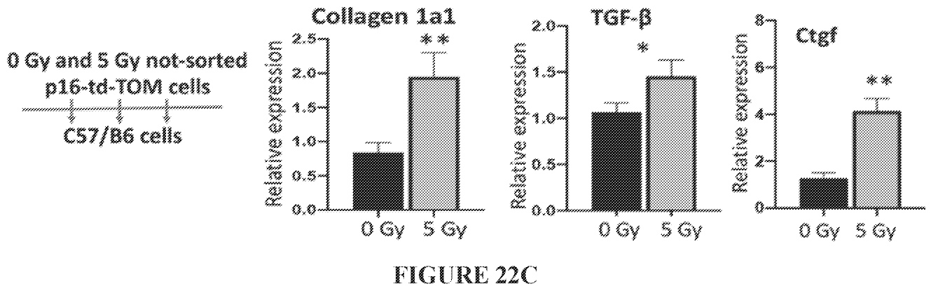

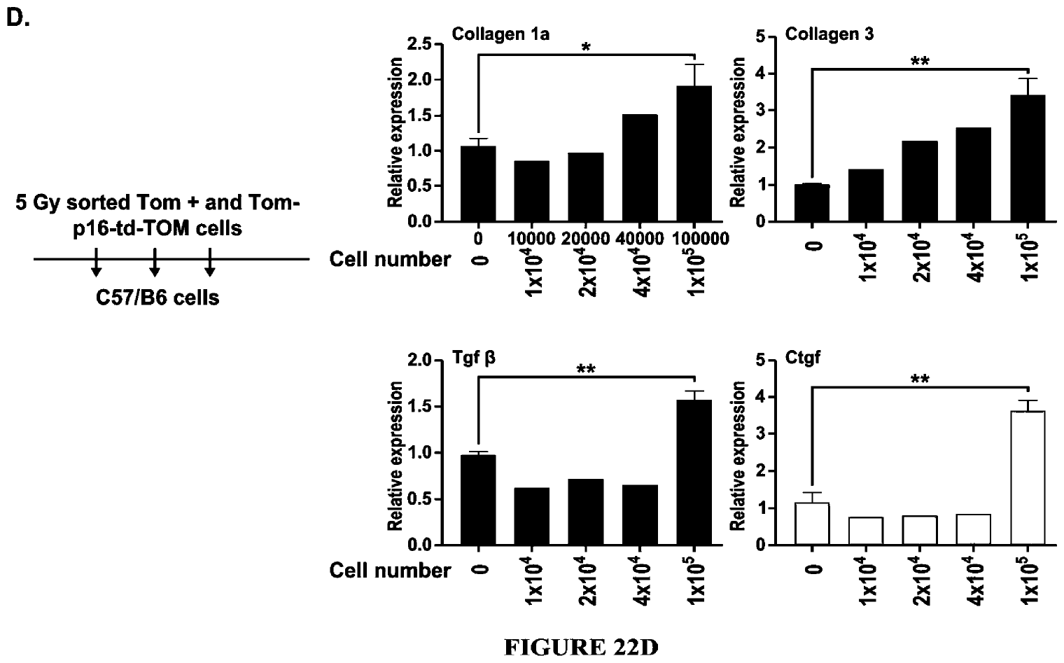

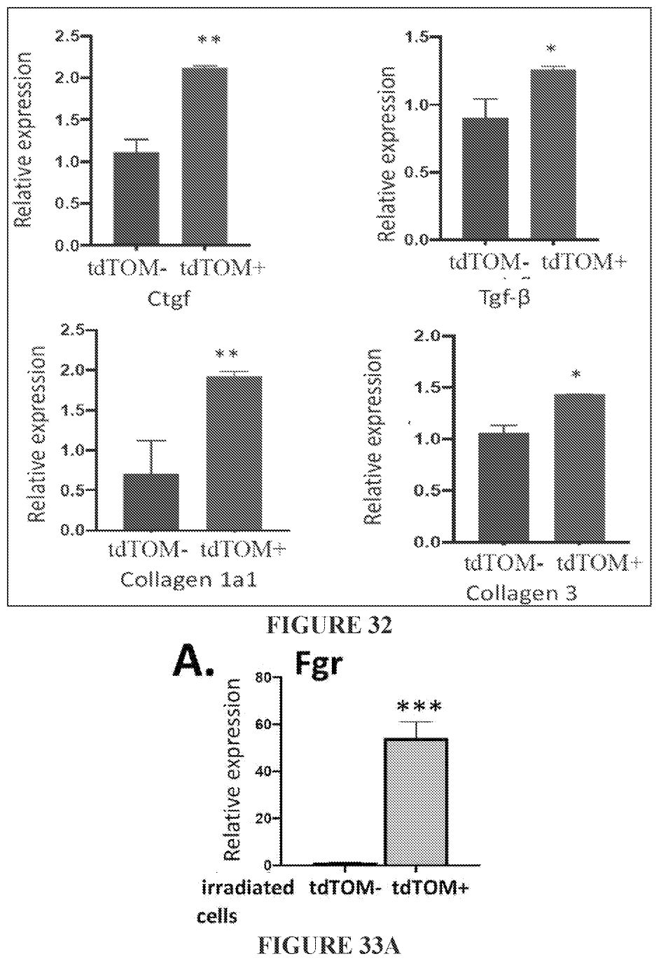

Senescent cells induce biomarkers of fibrosis in target cells in transwell culture. To determine whether humoral factors were released by senescent cells, induced biomarkers of fibrosis, the effects were tested on target cells in a transwell coculture system where target cells were separated from senescent cells by a 0.4-micron membrane (FIG. 22A). In the top chamber of each culture were plated irradiated, sorted tdTOM+ cells compared to irradiated, sorted tdTOM− cells with irradiated non-sorted cells. In the bottom layer of each culture either MSC target cells (FIG. 22B. FIG. 22C) or primary mouse tail fibroblasts (FIG. 32) were placed. Gene induction levels in target cells were quantitated from the bottom wells by using RT-qPCR. Upregulation of TGF-β, CTGF, Collagen 1, Collagen 3, and α-smooth muscle actin was observed in target cells by irradiated tdTOM+ cells compared to non-irradiated cells (FIG. 22A-FIG. 22D and FIG. 32).

To determine whether irradiation-induced induction of profibrotic genes in target cells by senescent cells was inhibited by the presence of irradiated non-senescent cells, unsorted irradiated cells were tested in the transwell cultures. These populations also induced biomarkers of fibrosis in target cells including TGF-β, CTGF, Collagen 1, Collagen 3, α-smooth muscle actin (FIG. 22C). These results establish that 9% of senescent cells were active in inducing biomarkers of fibrosis even in the presence of 90% non-senescent cells.

Next, the effect of cell number on the induction of biomarkers of fibrosis was quantitated. Sorted senescent tdTOM+ cells were plated in several numbers to determine the minimal number required to stimulate fibrotic gene expression in target cells. A minimum number of 104 senescent cells were required to induce a detectable induction of Collagen 1α, and Collagen 3 in target cells in the transwell cultures. This induction was elevated by increasing numbers of tdTOM+ cells in a direct dose-response relationship. A minimal number of 105 tdTOM+ cells was required to induce TGF-β and CTGF (FIG. 22D). The data indicate that purified irradiated tdTOM+ senescent cells induce biomarkers of fibrosis in target cells in a non-contact transwell system which is in line with the finding that TGF-β and collagen were also induced in the lungs of RIPF lungs (Epperly M W et al. Radiat Res. 2021. https://doi.org/10.1667/RADE-20-00286.1; Kalash R et al. Radiat Res. 2013, 180, 474-90).

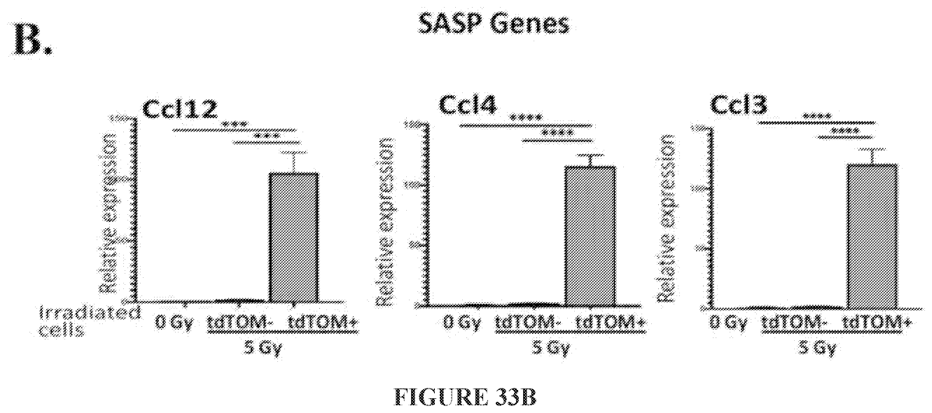

Tyrosine kinase Fgr in senescent cells induces biomarkers of fibrosis. Next, it was confirmed that upregulation of Fgr in senescent cells, as determined by RNA-seq correlated with overexpression of Fgr by RT-qPCR (FIG. 33A). Also, levels of SASP genes including Cc13, Ccl12, and Ccl4 that were induced by radiation in senescent cells were measured (FIG. 33B).

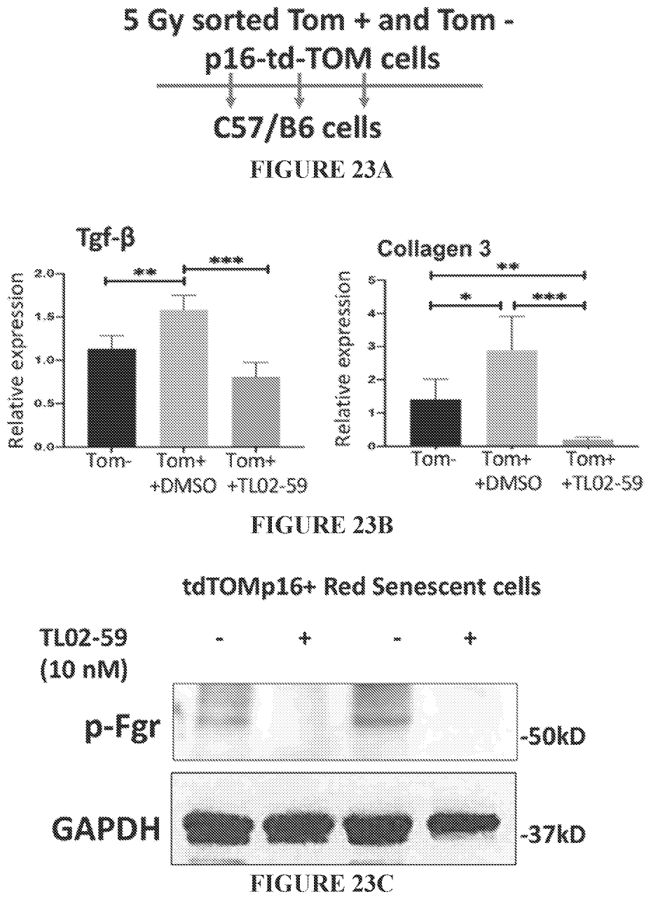

The role of Fgr in fibrosis induction was next investigated by two methods of inhibition: first, pharmacologic inhibition of Fgr was applied by adding the Fgr-specific small-molecule inhibitor TL02-59 (FIG. 23A, FIG. 23B). Parallel KINOMEscan analysis, in vitro kinase assays, target kinase gene expression profiling, and efficacy studies identified the Src-family kinase Fgr as the primary target for TL02-59 (Weir M C et al. ACS Chem Biol. 2018, 13, 1551-9). Using the transwell non-contact coculture system, the effect of a specific inhibitor of Fgr TL02-59 (10 nM) on the induction of biomarkers of fibrosis in target cells by tdTOM+ senescent cells was measured for 10 days. The cells were treated with 10 nM TL02-59 following a published protocol (Weir M C et al. ACS Chem Biol. 2018, 13, 1551-9). It was determined by RT-qPCR that TL02-59 significantly inhibited induction of collagen 3 and TGF-beta (FIG. 23B). It was confirmed that TL02-59 inactivated Fgr by phosphorylation in senescent cells using western blotting and a phosphor-Fgr antibody (FIG. 23C).

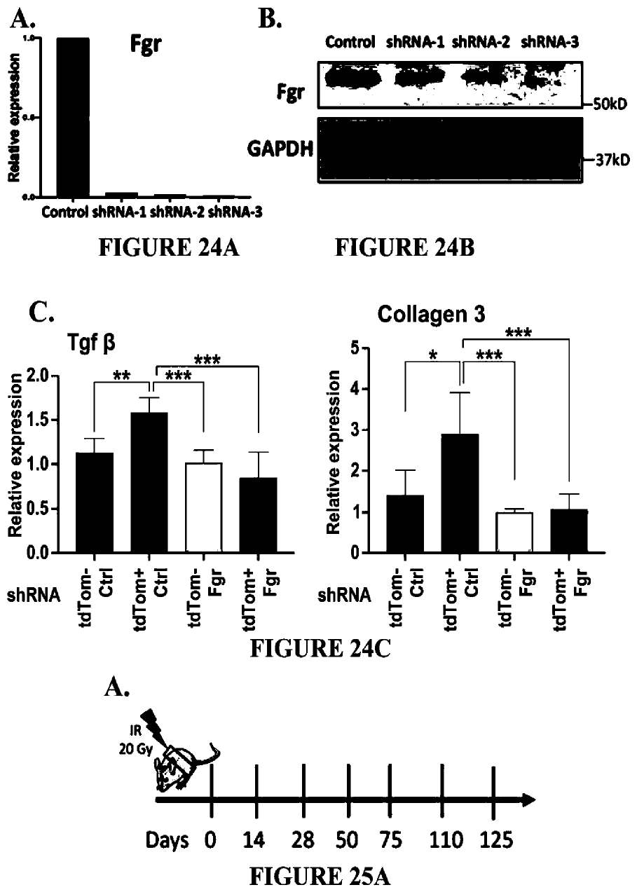

shRNA silencing of Fgr does not block irradiation-induced senescence but suppresses induction of fibrosis biomarkers in target cells. The Fgr inhibitor, TL02-59, might affect both the senescent cells on the top of the transwell well and the target cells at the bottom well in the transwell. Next, a stable Fgr shRNA expressing subline of the MSC cell line was constructed. Candidate-Fgr shRNAs were tested by transient transfection into tdTOMp16 bone marrow stromal cells and efficacy was measured after 72-h by RT-qPCR and western blots (FIG. 24A, FIG. 24B). shRNAs were selected that produced over 80% Fgr silencing and derived stable cell lines by puromycin resistance. It was then tested whether irradiated Fgr-silenced cells became senescent. At 10 days after 5 Gy irradiation, there was no detectable reduction in the 7-10% cell senescence by Fgr shRNA knockdown (silenced) in tdTOMp16 cells compared to control tdTOMp16 cells. Furthermore, RT-qPCR showed, p16 and p21 levels were increased in irradiation-induced shRNA knockdown senescent cells. However, Fgr-silenced tdTOMp16 red senescent cells did not induce TGF-β or collagen 3 (biomarkers of fibrosis) in target cells after 10 days in transwell cultures (FIG. 24C).

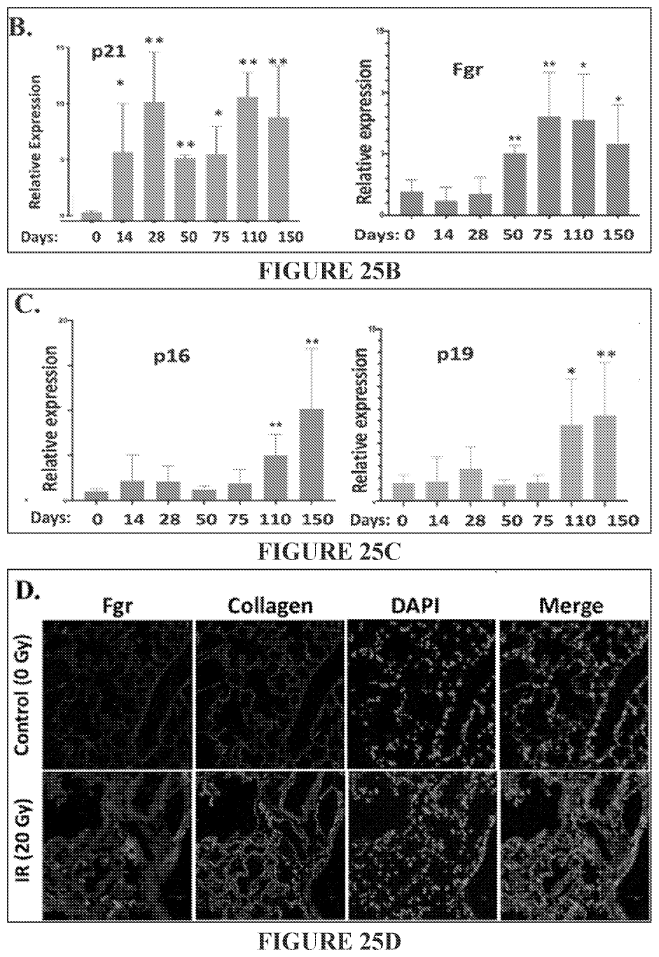

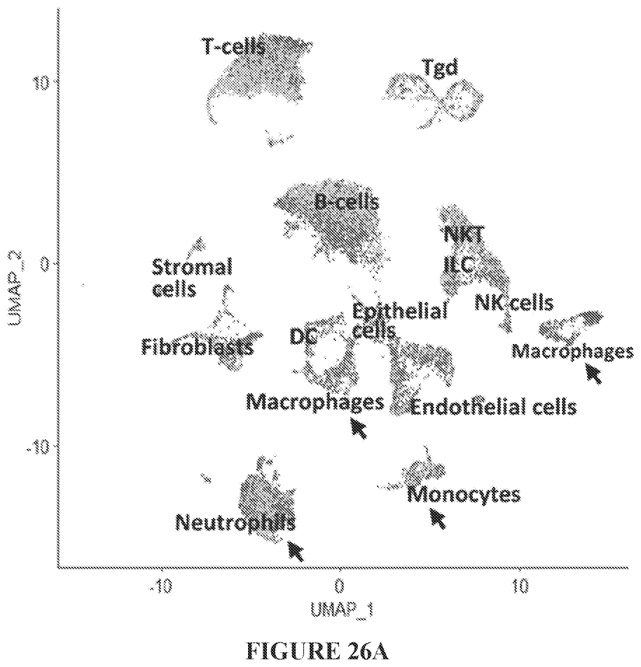

Lung irradiation induces Fgr in senescent cells prior to detection of radiation-induced pulmonary fibrosis. Prior studies showed senescent cells were detected by β-gal and p16 staining of lungs at day 75 after 20 Gy thoracic irradiation and before detection of fibrosis at day 150 (Epperly M W et al. Radiat Res. 2021. https://doi.org/10.1667/RADE-20-00286.1). Levels of Fgr compared with p21, p16, and p19 in the lungs of 20 Gy thoracic-irradiated mice were quantitated over 150 days (FIG. 25A). Fgr was upregulated in 20 Gy irradiated lungs at day 50 (FIG. 25B), while p16 and p19 were increased at 110 days (FIG. 25C), before detectable fibrosis at day 150. Both Fgr and collagen were significantly induced in the fibrotic lungs at day 150 after irradiation compared to no-irradiation control lungs as evidenced by immunohistochemistry (FIG. 25D).

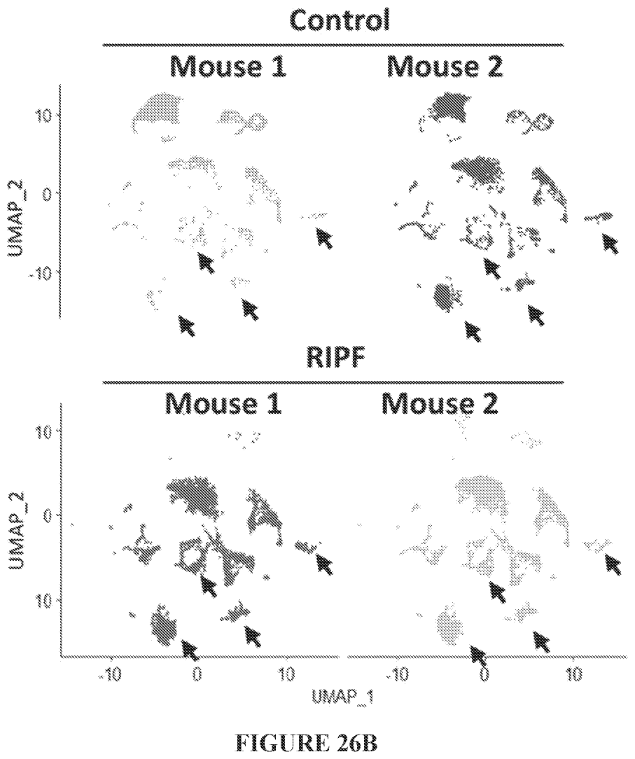

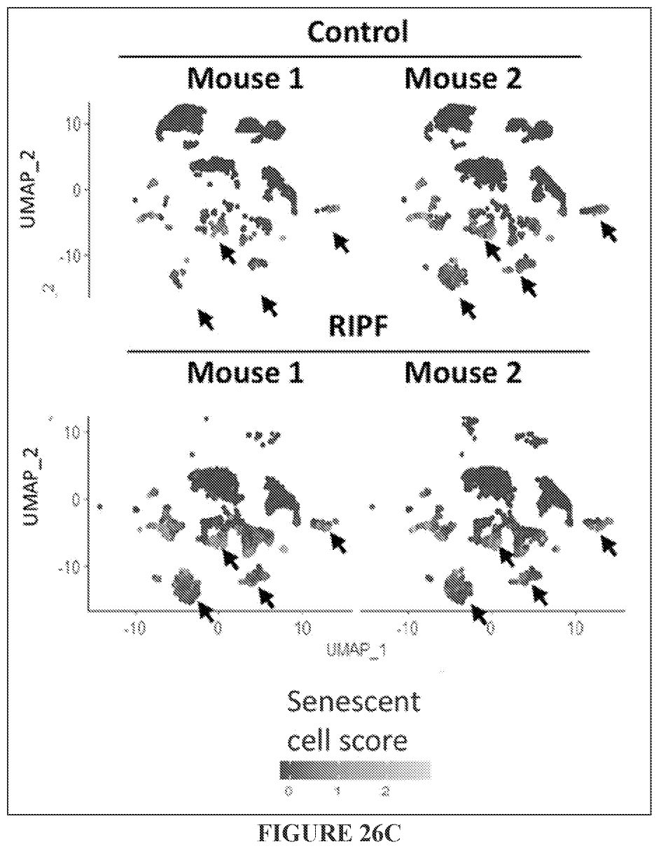

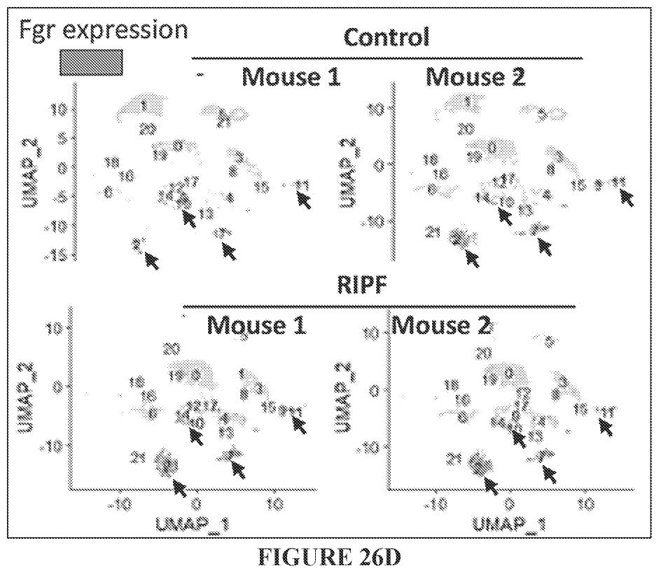

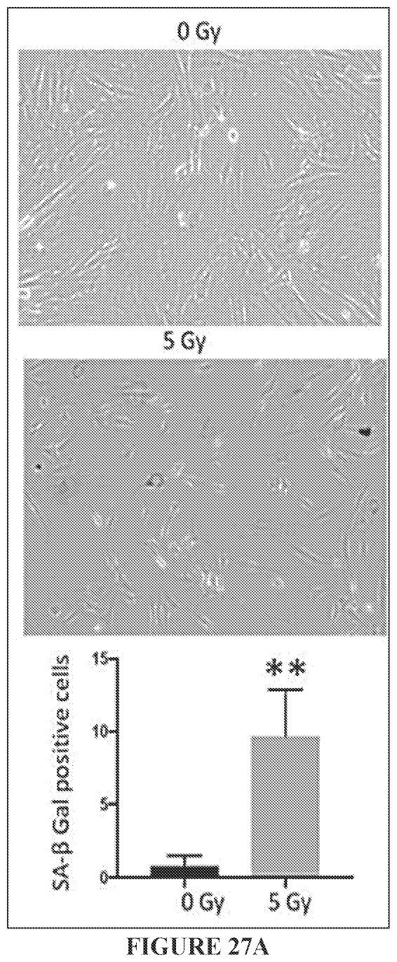

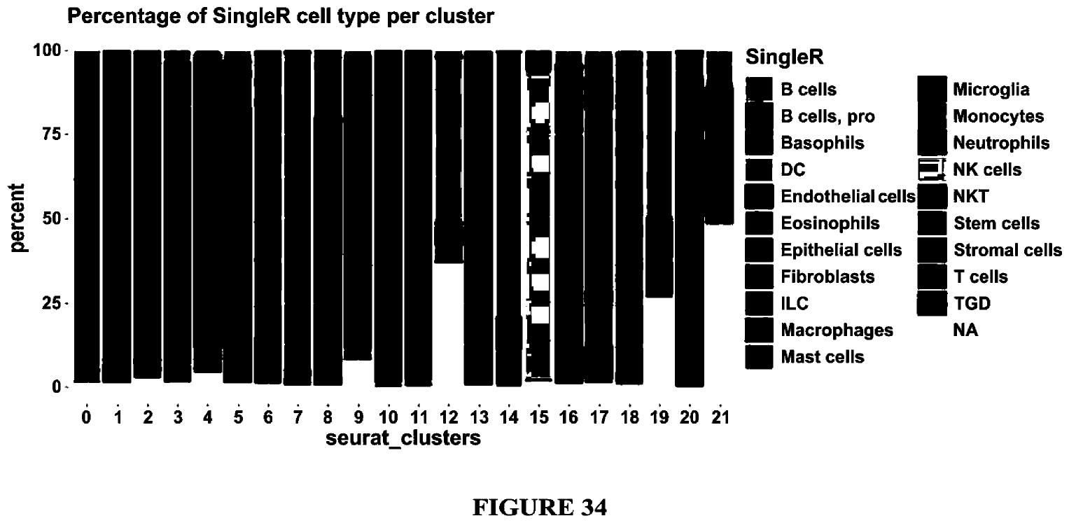

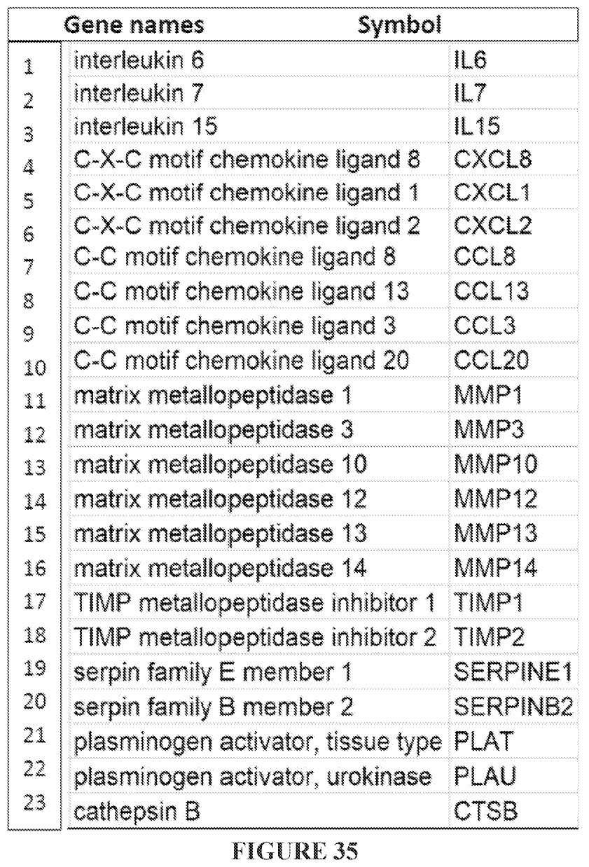

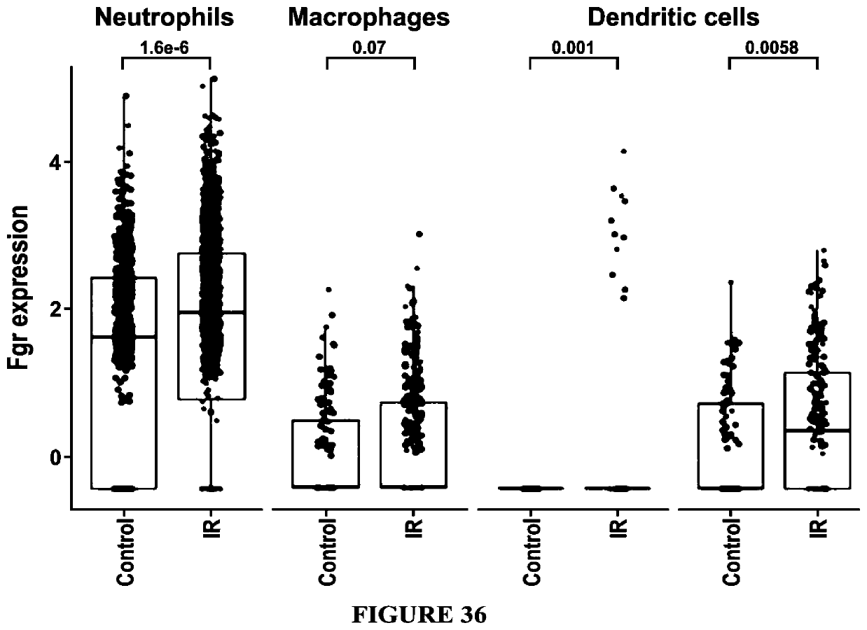

Single-cell RNA sequencing (scRNAseq) identifies Fgr induction in monocytes, macrophages, and neutrophils in thoracic-irradiated lungs. To identify the cell phenotypes that overexpressed Fgr in radiation-induced pulmonary fibrosis, scRNAseq was performed on cells from irradiated mouse lungs. Thoracic-irradiated mice were sacrificed at 150 days, lungs removed, and single cells isolated. A total of 19879 cells were used for integrated single-cell RNA-seq analysis. Cell types were assigned to each cluster based on the expression of established markers from the LungMAP and IMMGen databases (FIG. 34). There were identifiable epithelial cells, endothelial cells, macrophages, monocytes, neutrophils, dendritic cells, T cells, natural killer cells, B cells, stromal cells, and fibroblasts (FIG. 26A, FIG. 26B). The cell phenotypes in two control mice and two 150-day irradiated mice are shown. Using a panel of 23 senescence-associated genes (FIG. 35), senescence markers were detected in macrophages and neutrophils (FIG. 26C). ScRNAseq analysis revealed Fgr was present in monocytes, neutrophils in control lungs. At 150 days after 20 Gy irradiation, Fgr was significantly upregulated in neutrophils, a cluster of senescent cell macrophages, and in dendritic cells (FIG. 26D and FIG. 36).

### Discussion

In these studies, radiation-induced senescent cells were purified. A cell line was derived from tdTOMp16+ bone marrow in which the red tomato (tdTOM) fluorochrome is attached to exon1 of one allele of the p16 gene while the other allele is uninterrupted (Sessions G A et al. FASEB J. 2019, 33, 12364-73). Cells irradiated to 5 Gy, showed 7-10% senescent cells at day 10. Irradiation induces DNA-damage-driven senescence including upregulation of CDK1 p16 (Li M et al. Front Pharm. 2018, 9, 522).

RIS (tdTOM+) cells were compared with non-senescent (tdTOM−) cells in the same cultures. Senescent cells were clearly different from irradiated non-senescent cells and control non-irradiated cells by RNA-seq. PCA and the heat maps indicated that levels of 23 profibrotic genes were increased in senescent cells including TGFbeta, Tgf-alpha, TIMP2, Fgf2, Fgfl 1, and Col24al. The tyrosine kinase Fgr was Log 2 6.17-fold upregulated in RIS cells, which induced fibrosis biomarkers in target cells, separated in transwell cultures. Inhibition of Fgr by shRNA knockdown or adding an inhibitor of Fgr to the cultures blocked the induction of fibrotic biomarkers.

Fgr kinase has been implicated in proinflammatory adipose tissue macrophage activation, diet-induced obesity, insulin resistance, and liver steatosis (Acin-Perez R et al. Nat Metab. 2020, 2, 974-88). Fgr is known to be induced in some patients with acute myeloid leukemia, and inhibition of Fgr by TL02-59 clears transplanted leukemic cells in mice (Weir M C et al. ACS Chem Biol. 2018, 13, 1551-9). Tyrosine kinase activity is known to disturb physiological homeostasis and lead to cancer, vascular disease, and fibrosis. In fibrosis, receptor tyrosine kinases like PDGF receptor (PDGFR), VEGF receptor (VEGFR), EGF receptor (EGFR), and JAK kinases and nonreceptor tyrosine kinases such as e.g., c-Abl, c-Kit, and Src kinases have been identified as determinants of disease progression and potential targets for anti-fibrotic therapies (Yilmaz O et al. Growth Factors. 2015, 33, 366-75; Ang X et al. Biochem Cell Biol. 2018, 96, 742-51; Xu M et al. Nat Med. 2018, 24, 1246-56; Van Deursen J M. Nature. 2014, 509, 439-46). Specifically. Fgr deficiency has been linked to the reduced secretion of chemokines in the lungs in response to lipopolysaccharides (Mazzi P et al. J Immunol. 2015, 195, 2383-95; Davalli P et al. Oxid Med Cell Longev. 2016, 2016, 3565127).

Senescent cells produce reactive oxygen species (ROS) (Acin-Perez R et al. Cell Metab. 2014, 19, 1020-33), which activate the tyrosine kinase Fgr. Fgr upregulation was identified in irradiated mouse lungs, and the increase was detected at day 50, significantly before detection of fibrosis at day 150. By scRNAseq from mouse lungs, it was shown that Fgr is predominantly expressed in macrophages, monocytes, and neutrophils. In radiation-induced pulmonary fibrosis, macrophage senescence was observed and a distinct cluster of macrophages appears where Fgr is upregulated. At present, it is not known if Fgr is unique from other tyrosine kinases with respect to the induction of radiation-induced pulmonary fibrosis (Acin-Perez R et al. Nat Metab. 2020, 2, 974-88). Ablation of Fgr is known to impair proinflammatory macrophage polarization (Acin-Perez R et al. Cell Metab. 2014, 19, 1020-33). In prior studies, using scRNAseq, four separate macrophage clusters were present in idiopathic pulmonary fibrosis patient's lungs. Two macrophage clusters were represented equally in normal and fibrotic lungs, while two other clusters were found only in the lungs of idiopathic pulmonary fibrosis patients (Livak K J et al. Methods. 2001, 25, 402-8). Senescent fibroblasts and type II airway epithelial cells and macrophages have been reported to accumulate in the lungs during the induction of radiation-induced pulmonary fibrosis (Kasmann L et al. Radiat Oncol. 2020, 15, 214; Kasmann L et al. Radiat Oncol. 2020, 15, 214; Su L et al. Cell Death Dis. 2021, 12, 527). Fgr is known to be upregulated in the lungs in idiopathic pulmonary fibrosis (IPF), and is primarily expressed in macrophages, monocytes, and dendritic cells (Adams T S et al. Sci Adv. 2020, 6, eaba 1983). The data herein is consistent with prior publications emphasizing the role of monocyte/macrophages in lung fibrosis and extend the data to radiation-induced pulmonary fibrosis. Future studies can elucidate whether Fgr is involved universally in all kinds of senescence-mediated fibrosis.

Senescence may impair healing via the CXCR2 receptor, which, impedes repair (Justice J N et al. eBio Med. 2019, 40, 554-63; Wilkinson H N et al. Front Cell Dev Biol. 2020, 8, 773). A major ligand of CXCR2 is Cxcl2, which is a SASP component. In the RNA-seq analysis, Cxcl2 was induced in senescent cells. Since SASP recruits macrophages to sites of lung injury, such cells may impair tissue healing (Kasmann L et al. Radiat Oncol. 2020, 15, 214; Amor C et al. Nature. 2020, 583, 127-32). SASPs can induce paracrine senescence in neighboring cells; thereby forming the generation of reactive oxygen species (ROS), and the DNA-damage response (Correia-Melo C et al. Longev Healthspan. 2014, 3, 1). Paracrine senescence may exacerbate the biliary injury and impair regeneration in the liver (Ferreira-Gonzalez S et al. Nat Commun. 2018, 9, 1020). Targeted reduction of SASP, through TGF-β inhibition helps restore hepatocyte proliferation, decreases fibrosis, and improves, overall, liver function (Bird T G et al. Sci Transl Med. 2018, 10, eann1230).

Removal of senescent cells to prevent late radiation fibrosis may seem logical (Okuda R et al. J Thorac Dis. 2019, 11, 857-64; Kalash R et al. Radiat Res. 2013, 180, 474-90; Kirkland J L et al. J Intern Med. 2020, 288, 518-36; Cazzola M et al. Expert Opin Investig Drugs. 2018, 27, 573-81; Zhu Y et al. Aging Cell. 2015, 14, 644-58); however, senolytic drugs may not target all senescent cells (Ang X et al. Biochem Cell Biol. 2018, 96, 742-51; Xu M et al. Nat Med. 2018, 24, 1246-56; Van Deursen J M. Nature. 2014, 509, 439-46). Senolytic drugs Dasatinib plus Quercetin in idiopathic pulmonary fibrosis patients showed no changes in pulmonary function and had unclear effects on reducing SASP (Justice J N et al. eBio Med. 2019, 40, 554-63). Dasatinib promotes apoptosis (Vitali R et al. Int J Cancer. 2009, 125, 2547-55) by inhibiting Src kinases (Kirkland J L et al. J Intern Med. 2021, 288, 518-36). Quercetin (Yousefzadeh M J et al. EBioMedicine. 2018, 36, 18-28), a naturally occurring flavonoid acts in part by inhibiting BCL-2 family members. While Dasatinib plus Quercetin is effective in preventing aging in mouse models, neither alone are senolytic because of the complexity of their targets (Yilmaz O et al. Growth Factors. 2015, 33, 366-75; Yousefzadeh M J et al. EBioMedicine. 2018, 36, 18-28).

Alternatively, retaining senescent cells may provide valuable functions including restraining tumor growth (Schosserer M et al. Front Oncol. 2017, 7, 278), stimulating wound healing (Wilkinson H N et al. Front Cell Dev Biol. 2020, 8, 773), and promoting tissue repair (Yun M H. Int J Dev Biol. 2018, 62, 591-604).

If removal of specific components of SASP can be achieved, it is possible that retaining senescent cells may help restore and repair fibrotic lungs from radiation damage.

Other approaches to treat irradiation pulmonary fibrosis include pirfenidone and nintedanib, which slow the progression of fibrosis in the lung (Hewitt R J et al. Drugs Aging. 2019, 36, 485-92; Paliogiannis P et al. Curr Med Chem. 2021, 28, 2234-47). Pirfenidone works through the inhibition of TGF-β (Stahnke T et al. PLoS ONE. 2017, 12, e0172592). Nintedanib is a tyrosine kinase inhibitor for fibroblast growth factor receptor (FGFR)-1 and vascular endothelial growth factor receptor (VEGFR) (Sato S et al. Respir Res. 2017, 18, 172). The efficacy of these agents is poor (Fukihara J et al. Expert Rev Respir Med. 2016, 10, 1247-54; Behr J et al. Lancet. Respir Med. 2021, 9, 476-86) and their effects on senescent cells have not been studied in detail.

These studies identified a mechanism of radiation-induced pulmonary fibrosis mediated by increases in profibrotic Fgr in senescent cells. It was demonstrated that a small-molecule inhibitor of tyrosine kinase Fgr blocks the radiation fibrosis pathways. Studies evaluating the reduction of radiation-induced pulmonary fibrosis by TL02-59 treatment of thoracic-irradiated mice are in progress. Senescent cells and their induction of tyrosine kinase Fgr may play roles in lung fibrosis originating from causes other than radiation-induced pulmonary fibrosis. The therapeutic potential of inhibitors of Fgr in radiation-induced pulmonary fibrosis and other causes of lung fibrosis including after COVID-19 infection merit further study.

### Materials and Methods

Mice. The mouse strain tdTOMp16+ was obtained from Dr. B. O. Diekman. University of North Carolina at Chapel Hill (Sessions G A et al. FASEB J. 2019, 33, 12364-73), and C57BL/6 mice were purchased from The Jackson Laboratory. Thoracic irradiation was carried out according to published methods (Sivananthan A et al. Radiat Res. 2019, 191, 139-53). For the mouse thoracic irradiation experiment, a total of 34 C57BL/6 mice were randomly divided into control and experimental groups and sacrificed at indicated time points. All animal protocols used were approved by the University of Pittsburgh's IACUC and veterinary care was being provided.

Materials. Non-target control (sh-C018) and 3 shRNAs targeting mouse Fgr gene were purchased from (Sigma-Aldrich, St. Louis, MO) (shRNA #1, Clone ID: NM_010208.4-328s21c1. Sequence: CCGGACGGCTGAAGAACGCTATTTCCTCGAGGAAATAGCGTTCTTCAGCCGTTTTTT G (SEQ ID No:1); shRNA #2: Clone ID: NM_010208.2-758slc1, Sequence: CCGGGCGATCACAT AAAGCATTATACTCGAGTATAATGCTITATGTGATCGCTTTTT (SEQ ID No:2) and ShRNA #3, Clone ID: NM_010208.2-853slc1 Sequence. CCGGCGGCACTACATGGAAGTGAATCTCGAGATTCACTTCCATGTAGTGCCGTTTTT (SEQ ID No:3)), Rabbit anti-Fgr (#sc-74542) antibody was purchased from Santa Cruz Biotechnology, Inc and anti-phospho-Fgr antibody was purchased from the Invitrogen (Catalog #PA5-64583). Antibody to GAPDH was purchased from Sigma-Aldrich (St. Louis, MO). Antibody to collagen I was purchased from Abcam (Waltham, MA) (ab21286). TL02-59 was purchased from MedChemExpress as powder form (Cat. No.: HY-112852).

Long-term bone marrow cultures. Long-term bone marrow cultures were established according to previously published methods (Greenberger J S. Nature. 1978, 275, 752-4; Sakakeeny M A et al. J Natl Cancer Inst. 1982, 68, 305-17; Berhane H et al. Radiat Res. 2014, 181, 76-89). The contents of a femur and tibia of tdTOMp16+(heterozygous) mice (Sessions G A et al. FASEB J. 2019, 33, 12364-73) were flushed into a 40 cm. square plastic flask in Dulbecco's modified Eagle's medium containing 25% fetal calf serum and 10⁻⁵ M hydrocortisone sodium hemisuccinate and antibiotics. Cultures were medium-changed weekly with the removal of nonadherent cells and replacement with an equal volume of fresh medium. Cultures were maintained in a high humidity incubator with 7% CO₂ and medium-changed weekly. Stable shRNA knockdown cells were made by transfecting shRNA plasmids, no-target-control plasmids transfected by Lipofectamine 3000 and were selected against puromycin (4 μg/ml) as mixed clones.

Bone marrow stromal cell lines. Adherent cell monolayers from bone marrow cultures were maintained in Dulbecco's modified Eagle's medium supplemented with 20% fetal bovine serum and passaged weekly. Cell lines were derived from a 4-week culture harvest of the adherent layer and were maintained at 37° C. (Berhane H et al. Radiat Res. 2014, 181, 76-89).

Irradiation dose and time experiment. tdTOMp16+ bone marrow stromal cells were cultured in a 24-well plate at 50 and 100% confluency and the cells were exposed to 0, 1, 2.5, 5, and 7.5 Gy (n=4). For 10 days the tdTOM+ cells were imaged using Olympus BAX Motorized Fluorescence Microscope. The images were counted using ImageJ software.

In vitro cell sorting. Serial-passaged tdTPMp16+ bone marrow stromal cell line, either mock irradiated or 10 days after 5 Gy irradiation were sorted by FACS into tdTom− and tdTom+ populations using MoFlo XDP (Beckman Coulter) or FACSAria III (Becton Dickinson). For FACS gating, tdTOMp16+ Sorted cells were then used to (1) plate as single cells in each of the wells of 96-well plates RNA-seq experiment, or the functional studies using transwell coculture method section (Berhane H et al. Radiat Res. 2014, 181, 76-89).

Cell division monitoring: non-irradiated and 5 Gy Irradiated cells for both tdTOM+ and tdTOM− subpopulations were sorted. Cells were plated 10-day post irradiation as a single cell in each well of the 96-well plate (n=8-10). The cells were monitored for cell division for 4 weeks.

Stable shRNA knockdown of Fgr. tdTOMp16 cells were cultured at 70% confluency and 24 h after the cells were transfected with Lipofectamine 3000 reagent using non-target control and Fgr shRNAs. Transfected cell lines were sub-cultured after 24 h of transfection, and puromycin was added at 4 μg/ml concentration. The culture media of the transfected plates were replaced with fresh media to eliminate dead non-resistant cells. Once the resistant cells reach 80% confluency, cells were sub-cultured and the remaining cells were processed for Fgr knockdown validation by RT-q-PCR and western blotting.

Transwell coculture experiments. A Transwell system (0.4-μm pore size, polyester membrane; Corning, Kennebunk, ME) was used (Ejaz A et al. Stem Cells. 2019, 37, 791-802). C57BL/6 mouse bone marrow stromal cells or mouse primary tail fibroblasts were cultured in the bottom surface of a 9-cm² culture dishes. Above the adherent layer in the transwell, tdTOMp16+ bone marrow stromal cells (non-irradiated, irradiated non-sorted, irradiated, and sorted senescent tdTOM+, or irradiated non-senescent tdTOM− cells) were cultured in the upper transwell. For the TL02-59 experiment, the drug (10 nM) was added to the media at the time the cells were plated on the transwell. For the stable Fgr shRNA cells, non-target control and Fgr shRNA knockdown cell lines were also irradiated and sorted following the same protocol. Briefly, tdTOMp16+ cells were irradiated and after 10 days of radiation, the cells were FACS sorted for tdTOM+ and tdTOM− cells and 3×10⁵ cells added on the top wells in each case. Coculture was maintained for another 10 days. The target cells from the bottom well were lysed, and total RNA was isolated using TRizol (Sigma) reagent following the manufacturer's protocol. cDNA was synthesized, and expression of profibrotic genes (TGF-β1, CTGF, collagens (Col1, Col3), and α-SMA were analyzed using gene-specific TaqMan primers employing quantitative real-time polymerase chain reaction (PCR).

RNA isolation and cDNA synthesis. Total RNA was isolated from respective cell lines (tdTOMp16+ bone marrow stromal cell line, C57BL/6 bone marrow stromal cell line, and mouse primary tail fibroblasts) according to the protocol supplied with TRIZOLI Reagent (Invitrogen, Life Technologies, Thermo Fisher Scientific, Waltham, MA). The concentrations of the RNA samples were determined using a spectrophotometer and cDNAs were made from RNA (2 μg) using high-capacity RNA-to-cDNA™ Kit (Thermo Fisher Scientific) following the manufacturer's instructions.

Real-time PCR. Quantitative reverse transcription-PCR (qRT-PCR) was performed using Biorad CFX-connect Real-Time System instrument and commercially available target probes and Master mix (all from Applied Biosystems). Detection of mouse Fgr, p16 (CDKN2A), p21 (CDKNIA), p19, Collagens (1 and 3). TGF-beta, α-smooth muscle actin (Acta 2), CTGF and GAPDH were achieved using specific Taqman Gene Expression Assays (Mm00438951_m1, Mm00494449_m1, Mm04205640_g11, Mm0191861_m1, Mm01192933_g1. Mm01257348_m1, Mm00600638_m1. Mm00725412_s1, Mm00802305_g1, Mm99999915_g1, respectively). Real-time reactions were run using the following cycling parameters: 95° C. for 12 min, followed by 40 cycles of 95° C. for 15 s and 60° C. for 1 min. Differential gene expression was calculated by the ΔΔCT calculation (Livak K J et al. Methods. 2001, 25, 402-8).

RNA library construction and next-generation sequencing. RNA libraries were prepared from three groups of tdTOMp16+ cells consisting of: (1) non-irradiated cells (n=3); (2) irradiated senescent tdTOM+(red) cells (n=3); and (3) irradiated non-senescent tdTOM− cells (n=3). Compiled RNA-seq data was used in the analysis of FIG. 19A-FIG. 19C and FIG. 20A-FIG. 20C. To minimize the effect of any RNA degradation present in the samples, libraries for RNA-seq were generated from ribosomal RNA depleted total RNA rather than from mRNA isolated by poly-A selection. One microgram of each DNase I-treated RNA sample was used for library construction using the Illumina TruSeq Stranded Total RNA Library Prep Kit with Ribo-Zero Gold per the manufacturer's protocol. Adaptor-ligated fragments were amplified by PCR for nine cycles. The quality and size of the final library preparations were analyzed on an Agilent TapeStation. Sequencing was performed on the Illumina NextSeq. 500 NGS platform, generating ˜40 million paired-end 75 bp reads for each sample. The BioSample accession code for the deposited RNA-seq data to NIH is SAMN22069450.

Differential gene expression (DEG) detection and gene set enrichment analysis (GSEA) from RNA-seq data. The RNA-seq data were processed following previously established protocols (Bao R et al. Genome Med. 2020, 12, 90). Raw reads were assessed for quality using FastQC (v0.11.7) (Andrews S. Fast QC. A quality control application for high throughput sequence data. Babraham Institute. 2016, http://www.bioinformatics.babraham.ac. uk/projects/fastqc). Transcript-level abundance was quantified using Kallisto (v0.46.1) (Bray N L et al. Nat Biotechnol. 2016, 34, 525-7) in the reverse strand-specific mode with mouse reference assembly (GRCm38) and Gencode gene annotation (v25), and summarized into gene-level using tximport (v1.16.1) (Soneson C et al. F1000Res. 2015, 4, 1521). Lowly expressed genes (counts per million reads mapped (CPM)<1) were removed, and data were normalized across all samples using Trimmed Mean of M-values (TMM) method. Significant DEGs were identified using limma voom (v3.46.0) (Law C W et al. Genome Biol. 2014, 15, R29) with precision weights and filtered by fold change ≥2.0 or ≤−2.0 and FDR-adjusted P<0.05. GSEA was performed using R package fGSEA (v1.14.0) with gene sets of interest (chemotaxis, senescence, and profibrotic). Pathway enrichment of DEGs was detected using Ingenuity Pathway Analysis® (IPA) with Ingenuity Knowledge Base (QIAGEN, Inc).

Generation of single-cell suspensions from whole mouse lung. The lungs of mice were first inflated with 1 ml of sterile PBS and allowed to collapse, and then the lungs were inflated with the enzyme mix containing dispase (50 caseinolytic units/ml), collagenase (2 mg/ml), elastase (1 mg/ml), and DNase (30 ug/ml). The lungs were removed and immediately minced into small pieces (˜1 mm²). The tissue was transferred into 10 ml enzyme mix for enzymatic digestion for 30 min at 37° C. Enzyme activity was inhibited by adding 5 ml of phosphate-buffered saline (PBS) supplemented with 10% fetal calf serum (FCS) (Angelidis I et al. Nat Commun. 2019, 10, 963). Dissociated cells in suspension were passed through a 70-μm strainer and centrifuged at 500×g for 5 min at 4° C. Red blood cell lysis (Thermo Fisher 004333-57) was done for 2 min and stopped with 10% FCS in PBS. After another centrifugation for 5 min at 500×g (4° C.), the cells were counted using a Neubauer chamber and critically assessed for single-cell separation and viability. A total of 250,000 cells were aliquoted in 2.5 ml of PBS supplemented with 0.04% of bovine serum albumin and loaded for DropSeq at a final concentration of 100 cells/μl.

ScRNAseq. For scRNAseq, Chromium Next GEM Single Cell 3′ Reagent Kits v3.1 were used (10X Genomics, Pleasanton, CA) and the manufacturer's protocol was followed (Zheng G X et al. Nat Commun. 2017, 8, 14049). Briefly, cells are loaded onto a chip, run through the Chromium device, where they were encapsulated into individual oil droplets containing 10X Genomics' barcoded gel beads forming GEMs (Gel bead in Emulsion). These were then collected, and reverse transcription was performed, producing cDNA that contained the gene transcript with an associated cell barcode and unique molecular identifier. The cDNA was then purified, amplified, and libraries were prepared. Libraries were dual-indexed containing both a 17 and 15 index (Tabib T et al. J Invest Dermatol. 2018, 138, 802-10).

The quality of raw reads was assessed using FastQC. The feature-barcode count matrices were generated from FastQ files using the CellRanger pipeline (v6.0.1; 10X Chromium Single Cell 3′ V3 chemistry). CellRanger maps reads against mouse genome GRCm38 using STAR aligner, followed by Unique Molecular Identifier (UMI) counting with sequencing error correction and call barcode calling using the “Empty Drops” method (Lun A T L et al. Genome Biol.2019, 20, 1-9). Downstream analysis was performed using Seurat (v3.9.9.9024) to import feature-barcode hdf5 files, filter cells to keep those with 200-5000 features (genes), and mitochondria gene content <15%, keep genes present in >3 cells and normalize data by the “LogNormalize” method. A total of 19,879 cells and 25,319 genes were kept for analysis. Uniform Manifold Approximation and Projection (UMAP) (Becht E et al. Nat Biotechnol. 2019, 37, 38-44) was generated using Principal Component Analysis (PCA) dimension reduction with scaled data. Cell clusters were identified using a shared nearest-neighbor (SNN) modularity optimization-based clustering algorithm and visualized on UMAP. Cell type assignment was performed using SingleR (v1.2.4) (Aran D et al. Nat Immunol. 2019, 20, 163-72) with the mouse Immunological Genome Project (ImmGen) as the reference database. Annotated cell types were manually inspected using known immune markers. Cell cycle phases were scored using genes in the “cc.genes.updated.2019” object after converting human gene symbols to the mouse by biomaRt (v2.44.4). The BioSample accession codes for the deposited scRNAseq data to NIH are SAMN22071902, SAMN22071903.

Immunohistochemistry. C57BL/6 received either 0 or 20 Gy irradiation to the thoracic cavity. Thoracic-irradiated mice were sacrificed at day 150 post irradiation. Explanted tissues were frozen in optimal cutting temperature (OCT), sectioned and immunochemistry was performed using antibodies for Fgr (Santa Cruz Biotechnology. Inc #sc-74542) and collagen I (Abcam. ab21286). Five randomly selected images were captured in a blinded fashion from each section using fluorescent confocal microscopy.

Statistics. For the analysis of the percent of red cells, two-way ANOVA was used, where radiation dose, day, and their interaction are factors, followed by post hoc t tests. For the RT-qPCR analysis of gene expression, and for the western blot analysis, one-way ANOVA was used followed by post hoc t tests. For the other two group comparisons, two-sample t tests or Wilcoxon rank-sum tests were used, where appropriate. P values less than 0.05 were regarded as significant. In these exploratory analyses, P values were not adjusted for multiple comparisons.

DATA AVAILABILITY. RNA-seq and Single-cell RNA sequencing data that support the findings of this study have been deposited in SRA with the accession code PRJNA768885 and PRJNA768942.

### Example 6—Silica Induced Lung Fibrosis Is Associated With Senescence, Fgr, and Recruitment of Bone Marrow Monocyte/Macrophages

Abstract. Background/Aim: The role of senescence and bone marrow-derived cells in silica-induced pulmonary fibrosis is unknown. Materials and Methods: C57BL/6HNsd, p16^{+/LUC}, and tdTOMp16+ mice were intratracheally injected with 200 mg/kg crystalline silica or irradiated (20 Gy) to the thoracic cavity and followed for the development of lung fibrosis. Results: The p16^{+/LUC} mice demonstrated senescence by day 7 after silica exposure. C57BL/6 mice exposed to silica demonstrated upregulation of p16, p21, and tyrosine kinase Fgr by day 7, whereas thoracic irradiation induced p21 and Fgr by day 50 and p16 by day 110. Silica exposed GFP+ bone marrow chimeric C57BL/6 mice demonstrated senescent cells and gfp+/Fgr+ monocyte/macrophages in the lungs on day 21. The Fgr inhibitor TL02-59 abrogated monocyte/macrophages recruitment in in vitro transwell experiments. Conclusion: Both silica and radiation exposure induce senescence and upregulate tyrosine kinase Fgr for the recruitment of bone marrow-derived monocyte/macrophages and the development of pulmonary fibrosis.

Introduction. Silicosis is associated with black lung disease (coal miner's disease) and remains a significant cause of environmental lung disease. Studies of lung specimens from patients suffering from silicosis and other causes of pneumoconiosis have demonstrated that there is a sequence of events including pulmonary epithelial cell damage, an inflammatory response involving both alveolar and interstitial pulmonary macrophages, and increased numbers of alpha-smooth muscle actin positive cells in areas of fibrosis. This process, which replaces functioning lung tissue with fibrotic cells, often leads to fatal lung fibrosis.

The role of senescent cells in the etiology of pulmonary fibrosis is a current subject of interest. Senescent cells have been shown to accumulate in the lungs during evolution of several causes of fibrosis, but their role is unknown. Furthermore, the phenotypes of senescent cells, and their time course of appearance have not been documented in silicosis. In the present study, the time course of appearance of senescent cells in the lungs of crystalline silica injected p16^{+/LUC} mice was elucidated (Liu J Y et al. Proc Natl Acad Sci U.S.A. 116(7). 2603-2611, 2019), images were captured on live mice based on the expression of the luciferase reporter gene, which is linked to p16 gene promoter. It was also sought to elucidate both the phenotype and function of senescent cells in the lungs of tdTOMp16+ mice (Sessions G A et al. FASEB J 33(11): 12364-12373, 2019) in which p16+ senescent cells express a Tomato (red) reporter gene, which allows red cells to be sorted from explanted lungs. The results from the silicosis mouse model were corroborated with the radiation-induced pulmonary fibrosis model. The magnitude of bone marrow origin monocyte/macrophages recruited in the lungs was also determined using gfp+ bone marrow chimeric tdTOMp16+ mice (Epperly M W et al. Am J Respir Cell Mol Biol 29(2): 213-224, 2003). The results demonstrate the accumulation in the lungs of both bone marrow-derived monocyte/macrophages and neutrophils during the evolution of silicosis.

### Materials and Methods

Mice and animal care. C57BL/6J, p16^{+/LUC} (Liu J Y et al. Proc Natl Acad Sci U.S.A. 116(7): 2603-2611, 2019), tdTOMp16+(Sessions G A et al. FASEB J 33(11): 12364-12373, 2019), and C57BL/6 gfp+ (Epperly M W et al. Am J Respir Cell Mol Biol 29(2): 213-224, 2003) mice were maintained according to Institutional Animal Care and Use Committee (IACUC) protocols and housed at 4 per cage. Animals were fed standard laboratory chow and deionized water.

Animals were injected intratracheally with 200 mg/kg crystalline silica (Corning, Inc., Glendale, CA, USA) dissolved in 100 μl of PBS. Mice were imaged for senescence using a Xenogen IVIS Imaging System 200 Series (PerkinElmer, MA, USA), as described previously (Kalash R et al. Radiat Res 180(5):474-490, 2013; Epperly M W et al. Radiat Res 196(3): 235-249, 2021).

For lung irradiation, animals received 20 Gy single-fraction thoracic irradiation and were then maintained according to the IACUC recommended laboratory conditions. Mice were sacrificed at serial time points after thoracic irradiation (0, 50, 75, 110, and 125 days) (Epperly M W et al. Radiat Res 196(3): 235-249, 2021). Lungs were removed and representative lung lobes were tested by RT-qPCR for levels of detectable mRNA for p16, Fgr, and p21.

Evolution of silicosis and assays for senescence. The p16^{+/LUC} mice were imaged weekly using Xenogen IVIS Imaging System 200 Series (PerkinElmer) (Kalash R et al. Radiat Res 180(5):474-490, 2013) and p16 positive cells associated with activation of luciferase were visualized by scanning animals injected with luciferin substrate according to published methods (Kalash R et al. Radiat Res 180(5):474-490, 2013). Individual animals were scanned weekly. The C57BL/6J, and tdTOMp16+ mice were examined by histologic evaluation of explanted lung samples at serial times after crystalline silica injection. Staining of lung cells for collagen 1, alpha-smooth muscle actin, and p16 markers were assayed according to previously published methods (Epperly M W et al. Radiat Res 196(3): 235-249, 2021). Counter-staining of sections was carried out using an antibody for the senescence associated tyrosine kinase Fgr (Shen K et al. Sci Signal 11(553): eaat5916, 2018).

Preparation of gfp+ bone marrow chimeric mice. Recipient adult mice were irradiated to 8 Gy total body irradiation (TBI) and 24 h later injected through tail vein with 1×10⁶ gender mismatched gfp+ bone marrow (Epperly M W et al. Am J Respir Cell Mol Biol 29(2): 213-224, 2003). Bone marrow chimerism was documented by examination of the peripheral blood of recipients at serial time points, and those mice deemed to be chimeric had over 50% gfp+ leukocytes in the peripheral blood.

In vivo gfp+ bone marrow migration assay. One month prior to the injection of silica into the thoracic cavity, tdTOMp16+ mice were irradiated and transplanted with gfp+ mouse bone marrow. Silica injected mice were sacrificed on day 23 and single cells were isolated and processed for cell sorting. The relative percentage of red senescent cells sorted from control and silica treated gfp+ marrow chimeric mouse lungs was measured. Expression of Fgr was analyzed by qPCR in tdTOM+ senescent epithelial cells that were also CD45−, CD326+, and in tdTOM+ senescent alveolar macrophages that were also CD45+, F4/80+, CD11−. The relative percentage of bone marrow derived gfp+ monocyte/macrophage cells was quantitated by sorting from control and silica treated tdTOMp16+ chimeric mouse lungs.

In vitro transwell experiments. Senescent bone marrow derived irradiated (5 Gy, subconfluent cultures held for 10 days) tdTOMp16+ cells were sorted for TOM+ (red) color and placed in Transwell cultures. Two types of transwell experiments were carried out. For induction of fibrosis biomarkers in target cells, senescent cells were placed into the top chamber separated from target mesenchymal stem cells (MSCs) derived from the adherent layer of long term C57Bl/6 mouse bone marrow cultures in the bottom chamber by a 0.4-micron pore size membrane. For cell migration experiments, senescent cells were placed on the bottom of the chamber, and gfp+ bone marrow cells were placed in the top compartment of Transwell cultures separated by 3.0-micron pore membranes (Ejaz A et al. Stem Cells 37(6): 791-802, 2019). The transwells were maintained in complete Dulbecco's Modified Eagles Medium supplemented with 10% fetal calf serum. The migration of gfp+ cells through the filter to the bottom compartment was measured by imaging the bottom wells and counting gfp+ cells relative to numbers of senescent TOM+ (red) cells. The phenotype of migrating gfp+ cells was determined by immunohistochemical staining using antibodies to markers of monocytes/macrophages and other bone marrow derived cell phenotypes according to published methods (Ejaz A et al. Stem Cells 37(6): 791-802, 2019). The Fgr tyrosine kinase inhibitor TL02-59 has been reported (Shen K et al. Sci Signal 11(553): eaat5916, 2018; Li H et al. Biomed Pharmacother 127: 110183, 2020).

Cell migration assay. Irradiated bone marrow mesenchymal stem cells (stromal cells) (5.0 Gy) were sorted for tdTOM+ and tdTOM− cells and compared with non-irradiated cells. Each cell population was cultured in the bottom of transwells in the transwell culture system. The cell layer in the bottom chamber was separated from the top cell populations well by a 3-micron pore size membrane. Equal numbers of (10⁵) gfp+ marrow cells were added to the top chamber of each transwell. The Fgr inhibitor TL02-59 (10 nM) or RS504393 (Medchem Express, Mammoth Junction, NJ, USA) (10 μM), or the chemokine (C-C motif) ligand 2 (CCL2) inhibitor, which is a CCR2 chemokine receptor antagonist, was added to the media. At 24 h later, the migration of gfp+ bone marrow cells into the bottom of the wells were imaged and cells counted. At 48 h later, the cells from the bottom well were further analyzed by MoFlo XDP (Beckman Coulter) Fluorescent Activated Cell Sorter (FACS) for the phenotype of gfp+ cells that had migrated from the top chamber of each well.

Immunohistochemical staining of lungs for fibrosis, senescent cells, and Fgr positive cells. Lung specimens were explanted at serial times after crystalline silica injection and immunohistochemically stained for collagen by Masson's Trichrome staining, for senescence-associated Beta-Galactosidase, p16, p21. p19 (Epperly M W et al. Radiat Res 196(3): 235-249, 2021), and tyrosine kinase Fgr (Shen K et al. Sci Signal 11(553): eaat5916, 2018). Sections were counter-stained with a second monoclonal antibody for immunohistochemistry according to published methods (Epperly M W et al. Radiat Res 196(3): 235-249, 2021).

Phenotype of sorted cells from the lungs of silica treated mice. Cells were isolated from the lungs of silica treated mice including Ly6C′ monocytes, which may exert a proinflammatory role in tissue injury. Their impact after injuries is poorly defined. The C-C chemokine receptor 2, which is expressed on Ly6C^{hi} monocytes was used for phenotyping, since it is essential for extravasation and transmigration into injured tissues. A selective C-C chemokine receptor 2 antibody (Misharin A V et al. J Exp Med 214(8): 2387-2404, 2017; Misharin A V et al. Am J Respir Cell Mol Biol 49(4): 503-510, 2013) was used to count Ly6C^{hi} monocytes recruited into the lungs after silica exposure.

Immunostaining for monocyte/macrophages and neutrophils. At 28 days after the crystalline silica (200 mg/kg) injection into the thoracic cavity of mouse, lung cells were isolated from tdTOMp16+ gfp+ chimeric mice. Lung single cell populations were immunostained and sorted for tdTOM+ and gfp+ cells. Gfp+ cells were then further sorted for alveolar macrophages (CD45+, F4/80+, and CD11b−), interstitial macrophages (CD45+, F4/80+, and CD11b+), and tdTOM+ cells were further sorted for alveolar macrophages (CD45+, F4/80+, and CD11b−), interstitial macrophages (CD45+, F4/80+, CD11b+), epithelial cells (CD45−, CD326), and endothelial cells (CD45− and CD31). Cells from the lungs of non-irradiated C57BL/6 mice were sorted for alveolar macrophages (CD45+, F4/80+, and CD11b−), interstitial macrophages (CD45+, F4/80+, and CD11b+), epithelial cells (CD45−, CD326+), and endothelial cells (CD45− and CD31). The relative percentage of each cell type was quantified from each sample. CD11b+ and CD11b− cells were stained and quantitated according to published methods (Misharin A V et al. Am J Respir Cell Mol Biol 49(4): 503-510, 2013; Lowell C A. Mol Immunol 41(6-7): 631-643, 2004) CD31, CD11b and CD45 were purchased from B.D. Biosciences, (San Jose, CA, USA) and F4/80 and CD326 were purchased from Invitrogen, Thermo Fisher Scientific (Waltham. MA, USA).

RNA isolation and cDNA synthesis. Total RNA was isolated from cell lines and explanted cells (tdTOMp16+ bone marrow stromal cell line, C57BL/6 bone marrow stromal cell line, and mouse primary tail fibroblasts) according to the protocol supplied with TRIZOLI Reagent (Invitrogen, ThermoFisher Scientific). The concentration of the RNA samples was determined using a microplate spectrophotometer Epoch, BioTech (Winooski, VT, USA) and cDNAs were synthesized from RNA (2 μg) using high-capacity RNA-to-cDNA™ Kit (Thermo Fisher Scientific) following manufacturer's instructions.

Real-time PCR. Quantitative reverse transcription-PCR (qRT-PCR) was performed using a Biorad CFX-connect Real Time System instrument (Hercules, CA, USA), commercially available target probes and, Master mix (all from Applied Biosystems, Thermo Fisher Scientific). Detection of mouse Fgr, p16, Collagen 1 (CDKN2A), Transforming growth factor beta (TGF-β), α-smooth muscle actin (Acta 2), connective tissue growth factor (CTGF), and Glyceraldehyde 3-phosphate dehydrogenase (GAPDH) was achieved using specific Taqman Gene Expression Assay reagents (Mm00438951 m1, Mm00494449_m1, Mm01192933_g1, Mm01257348_m1, Mm00600638_m1, Mm00725412_s1, Mm00802305_g1, Mm99999915_g1, respectively). Real time reactions were run using the following cycling parameters: 95° C. for 12 min, followed by 40 cycles at 95° C. for 15 s, and 60° C. for 1 min. Differential gene expression levels were calculated by the ΔΔCT calculation.

Statistical evaluation. In the Fgr expression analysis, the qPCR analysis for the expression of TGF-β and Collagen 3, and the FACS analysis for gfp+ and CD11b+ cells, cell groups were compared with one-way ANOVA followed by the two-sided two-sample t-tests. For the other two group comparisons, two-sample t-tests or Wilcoxon rank sum tests were used where appropriate. p-Values less than 0.05 were regarded as significant. In these exploratory analyses, p-Values were not adjusted for multiple comparisons.

### Results

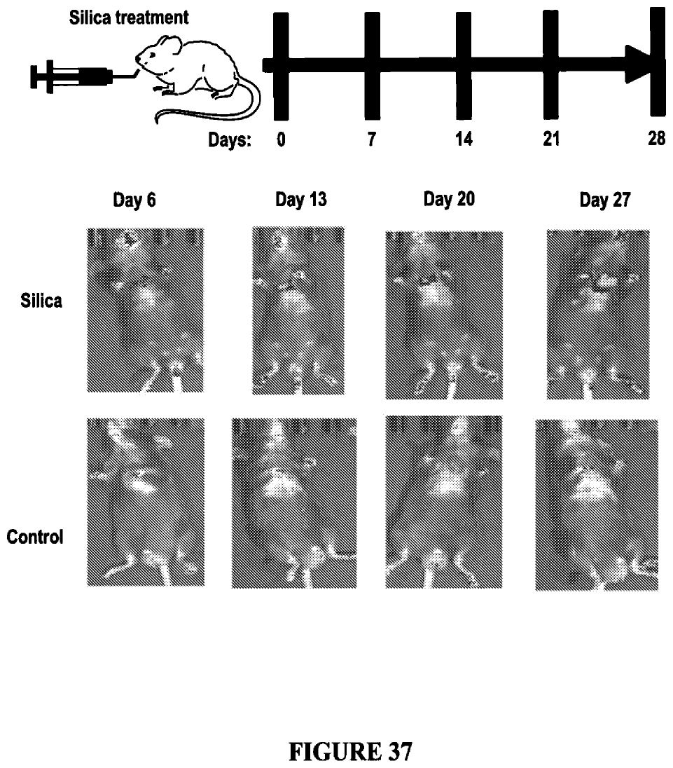

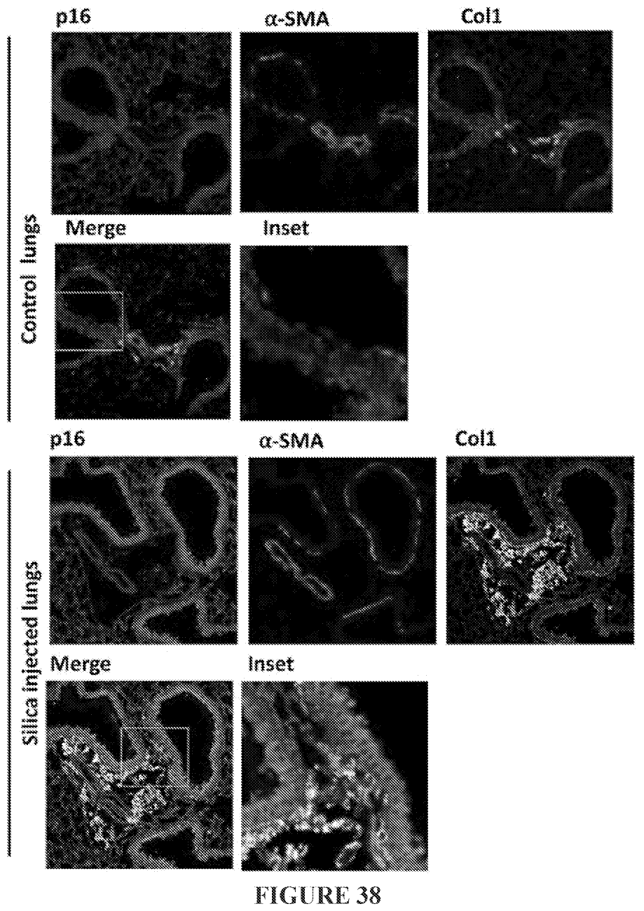

Crystalline silica induces senescence in mouse lungs. The appearance of senescent cells in the lungs of crystalline silica injected mice were correlated with the time of first appearance of fibrosis in the lungs of mice by first determining the time of appearance of p16+ cells in serial imaging of p16^{+/LUC} mice. As shown in FIG. 37, p16^{+/LUC} mice injected with crystalline silica first showed detectable p16+ areas, following luciferin injection, as early as day 6 and the p16+ area increased over time. In contrast, mice not exposed to crystalline silica showed no detectable luciferin-induced p16+ luciferase areas in the lungs.

Silica-induced epithelial senescence is associated with lung fibrosis. Next, the appearance of senescent cells in p16^{+/LUC} mice, which was first detected by IVIS imaging, were correlated with the appearance of p16+ senescent cells in C57BL/6J mice using histochemical staining of explanted lungs. Lungs were removed from C57BL/6J mice on day 28 after silica injection and immunostaining was performed for p16, alpha-smooth-muscle actin (α-SMA) and Collagen 1 (Col1). The epithelial lining of the silica injected mouse lungs showed increased p16 staining (FIG. 38, bottom panel) compared to the control lungs (FIG. 38, top panel) and revealed the presence of senescent cells. The p16 expressing cells of the lungs were juxtaposed to the cells expressing α-SMA, a marker for myofibroblasts. Col1 positive areas indicated the areas of fibrosis (FIG. 38).

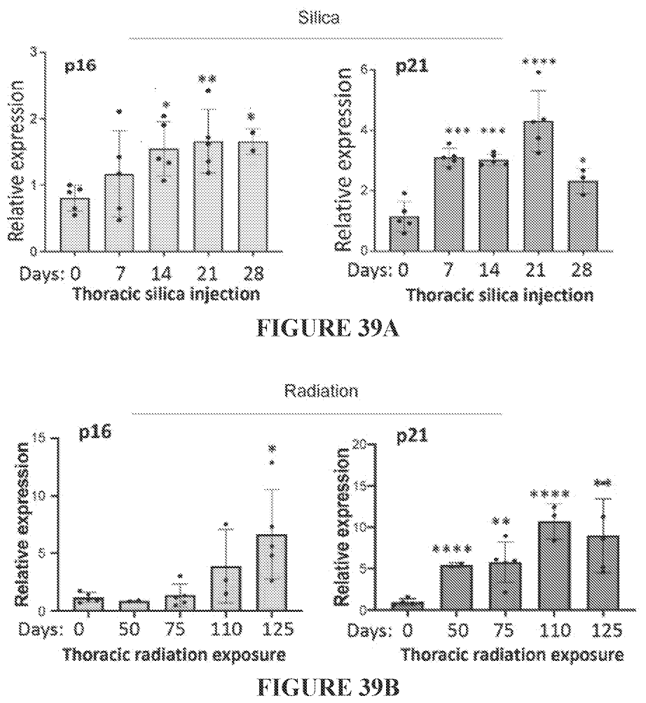

Senescence is detectable prior to pulmonary fibrosis in both cases of either silica-Induced or radiation-induced pulmonary fibrosis. Lungs were removed at serial time points from either silica-injected mice on days, 0, 7, 14, 21 and 28 (FIG. 39A), or from thoracic irradiated mice on days 0, 50, 75, 110 and 125 (FIG. 39B) and were analyzed for the expression of senescent biomarkers (p16 and p21) by qRT-PCR. In agreement with the earlier results (FIG. 37), senescent markers (p16 and p21) were upregulated in a time-dependent manner in both models of pulmonary fibrosis. Interestingly p16 expression was delayed in radiation-induced pulmonary fibrosis although it was expressed before fibrosis (FIG. 39B). These data confirm prior studies with thoracic irradiation showing that detection of significant numbers of senescent cells precedes the appearance of histologic fibrosis (Epperly M W et al. Radiat Res 196(3): 235-249, 2021), and clearly demonstrate the appearance of fibrotic areas after prior detection of senescence.

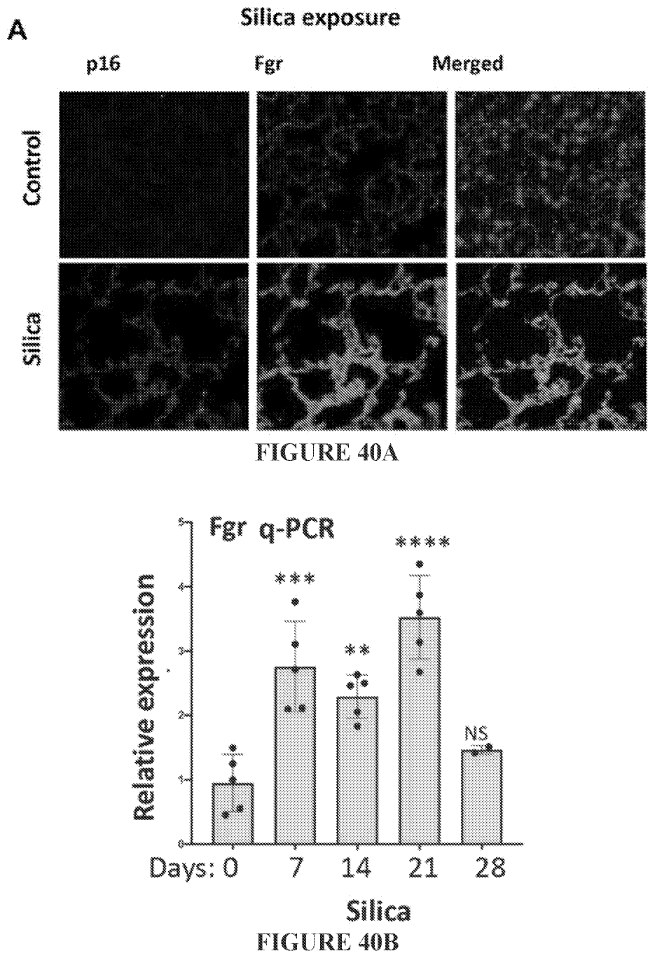

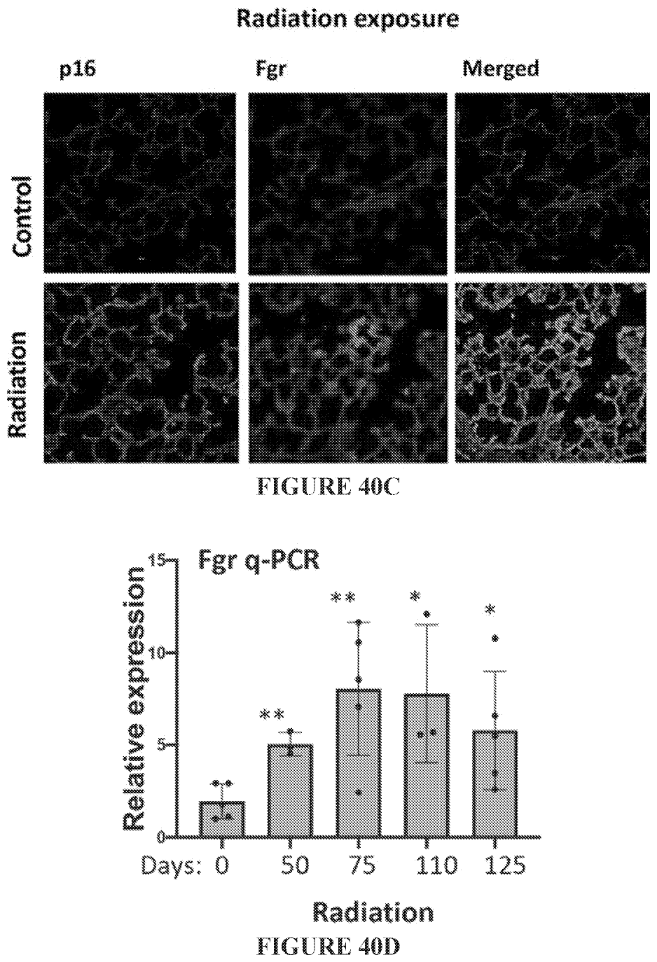

Tyrosine kinase Fgr is Induced prior to fibrosis in both cases of either silica- or radiation-induced pulmonary fibrosis. In human idiopathic pulmonary fibrosis, the tyrosine kinase Fgr is upregulated and since tyrosine kinases are known to contribute to pulmonary fibrosis (Okutani D et al. Am J Physiol Lung Cell Mol Physiol 291(2): L 129-L 141, 2006; Meng F et al. J Exp Med 185(9): 1661-1670, 1997; Acin-Perez R et al. Nat Metab 2(9): 974-988, 2020; Jing X et al. Acta Pharm Sin B 11(2): 394-405, 2021; Lowell C A et al. J Cell Biol 133(4): 895-910, 1996; Kovács M et al. J Exp Med 211(10): 1993-2011, 2014; Ingley E. Biochim Biophys Acta 1784(1): 56-65, 2008). Therefore, the expression of Fgr in either silica- or radiation-induced pulmonary fibrosis was evaluated next. Expression of Fgr was upregulated in both models of pulmonary fibrosis along with p16 (silica: FIG. 40A and radiation: FIG. 40C). To examine the expression of Fgr in a time-dependent fashion, either the silica-injected lungs on days, 0, 7, 14, 21 and 28 or the radiation-exposed lungs on days 0, 50, 75, 110 and 125 were analyzed by qRT-PCR. Compared to control, Fgr was significantly upregulated either on days 7, 14, and 21 for silica- or on days 50, 75, and 110 for radiation-induced pulmonary fibrosis (silica-FIG. 40B and radiation: FIG. 40D). Interestingly Fgr expression decreased at the last time point for both models.

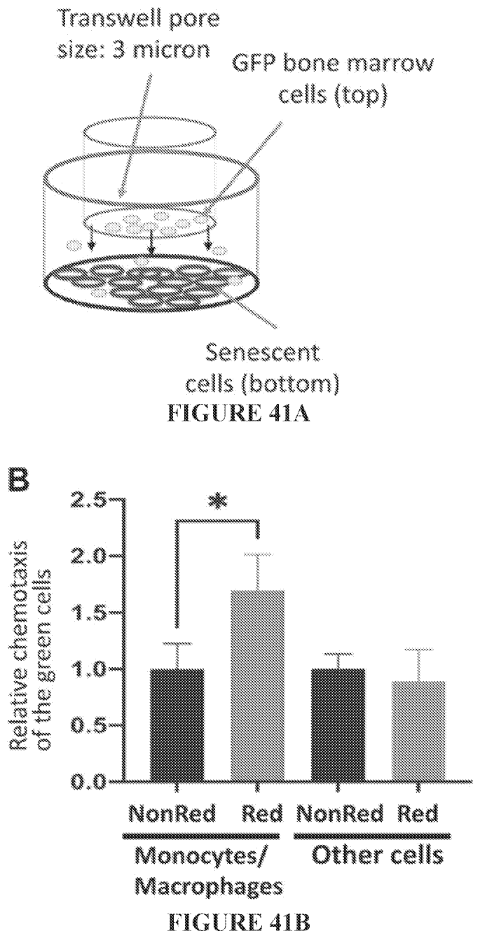

Bone marrow derived GFP+ monocytes/macrophages migrate towards radiation-Induced senescent cells in a transwell coculture system. Next, migration of freshly removed gfp+ bone marrow cells through the transwell filters in response to senescent cells placed in the bottom chamber of the transwells was quantitated. Gfp+ bone marrow cells were placed in the upper compartment of transwells separated by 3.0-micron filter from red senescent cells in the bottom chamber (FIG. 41A-FIG. 41B). The schematic for the chemotaxis in the transwell system is shown in FIG. 41A. Bone marrow derived gfp+ cells migrated through the 3.0-micron filters and were observed to accumulate in increasing numbers in the bottom chamber in close proximity to senescent cells. The migrating bone marrow cells were predominantly of the monocyte/macrophage phenotype but other phenotypes were also observed (FIG. 41B).

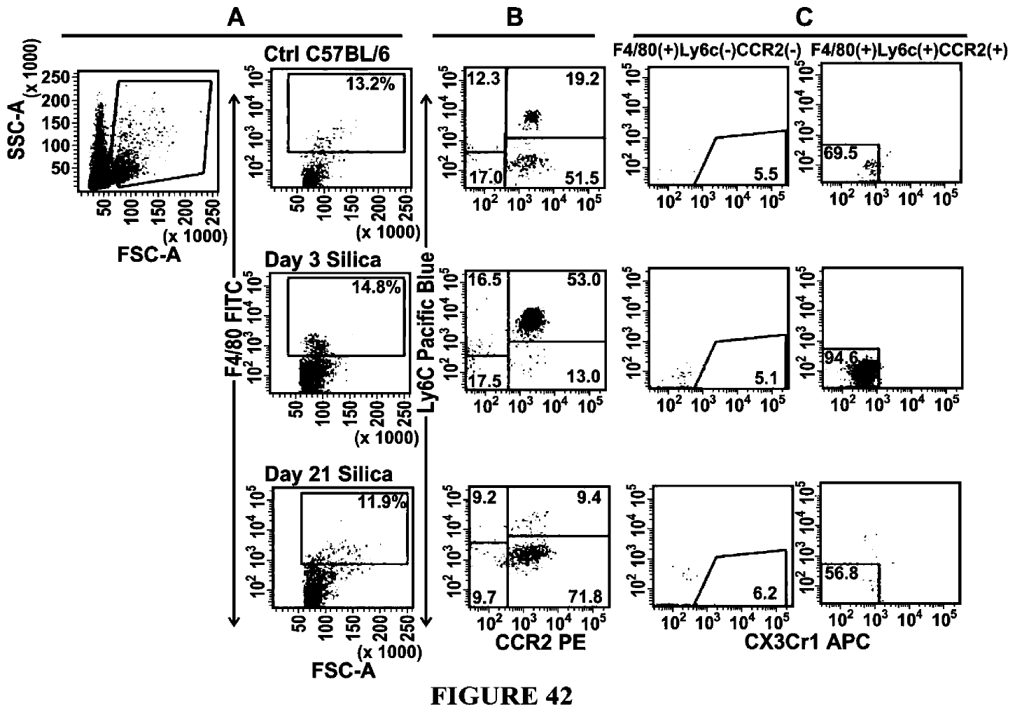

In vivo, bone marrow derived monocytes/macrophages migrate towards silicosis lungs. To examine whether in silicosis lungs there was monocyte/macrophage migration, FACS analysis of single cell suspensions of lungs from control or silica-treated mice on day 3 or day 21 was performed. F4/80 positive cells were analyzed (FIG. 42, panels in column A) for the expression of Ly6C and CCR2 and gated for 4 different populations; 1) Ly6C− & CCR2+ cells, 2) Ly6C^{hi} & CCR2+ cells, 3) Ly6C−& CCR2+ cells, 4) Ly6C^{hi} & CCR2− cells (FIG. 42, panels in column B). Ly6C is a marker to identify monocyte and macrophage subpopulations and F4/80+, CCR2+, Ly6C^{hi} designate inflammatory monocytes. A significant increase in F4/80+, CCR2+, Ly6C^{hi} cells (53%) was found in the lungs after only 3 days of thoracic silica injection compared to the control lungs (19.2%). Interestingly, on day 21 there was a steep reduction of F4/80+, CCR2+, Ly6C^{hi} cells (9.4%) and a robust increase in F4/80+, CCR2+, Ly6C− cells (71.8%) (FIG. 42, panels in column B). When the F4/80+, CCR2+, Ly6C^{hi} cells were further analyzed for the presence of the CX3CR1 marker (FIG. 42, panels in column C), as it is present in the inflammatory monocytes in circulation, majority of monocytes (94.6%) were CX3CR1 negative.

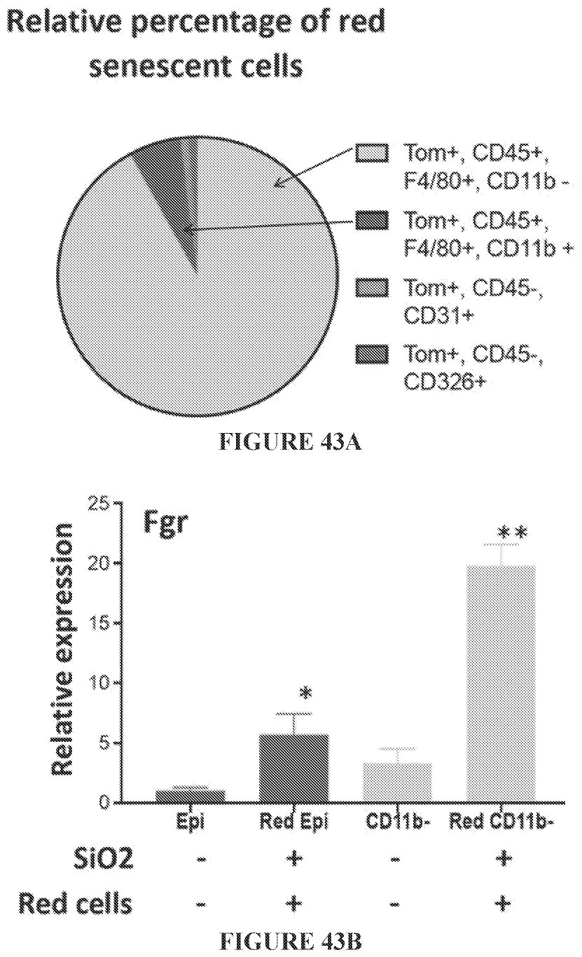

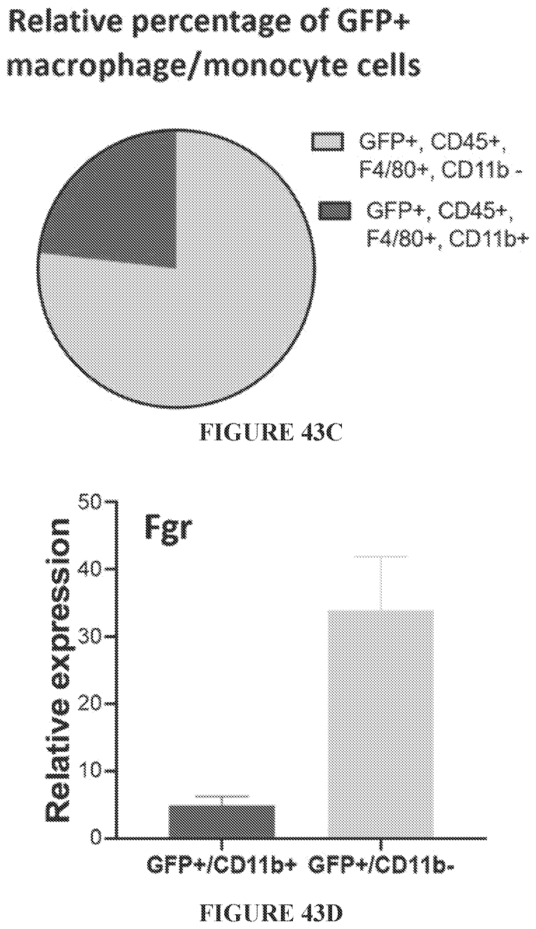

In silicosis lungs, tyrosine kinase Fgr expression is induced in senescent cells and in recruited bone marrow monocyte/macrophages. Next, the phenotype of migrating marrow cells to the silica-injected lungs in vivo was determined and the relative percentage of gfp+ monocyte/macrophages in previously bone marrow transplanted mice that were chimeric for gfp+ bone marrow was quantitated. Recipient tdTOMp16+ mice that were transplanted with gfp+ bone marrow and were chimeric by analysis of peripheral blood on day 50 after bone marrow transplantation were administered with crystalline silica. As shown in FIG. 43A-FIG. 43D, there were significant numbers of bone marrow derived gfp+ monocyte/macrophages in the lungs of tdTOMp16+ mice that had been injected with crystalline silica 23 days previously. CD45+, F4/80+, CD11b− and CD45+, F4/80+, CD¹¹b+ cells were found in both transplanted GFP+ bone marrow cells that had migrated to the recipient lungs as well as in GFP− cells that are native to the recipient lungs. CD₁₁b− population is designated as alveolar macrophages and CD11b+ cells are designated as interstitial macrophages (FIG. 43A-FIG. 43D). To evaluate the expression of Fgr in the lung cells 23 days after silica injection, qRT-PCR was performed after cell sorting. Baseline Fgr expression was significantly higher in alveolar macrophages compared to the lung epithelial cells and Fgr expression was induced in alveolar macrophages following treatment with silica. These data establish that bone marrow derived as well as native monocyte/macrophages contribute to the accumulation of inflammatory cells in the lungs of mice that have been injected with crystalline silica.

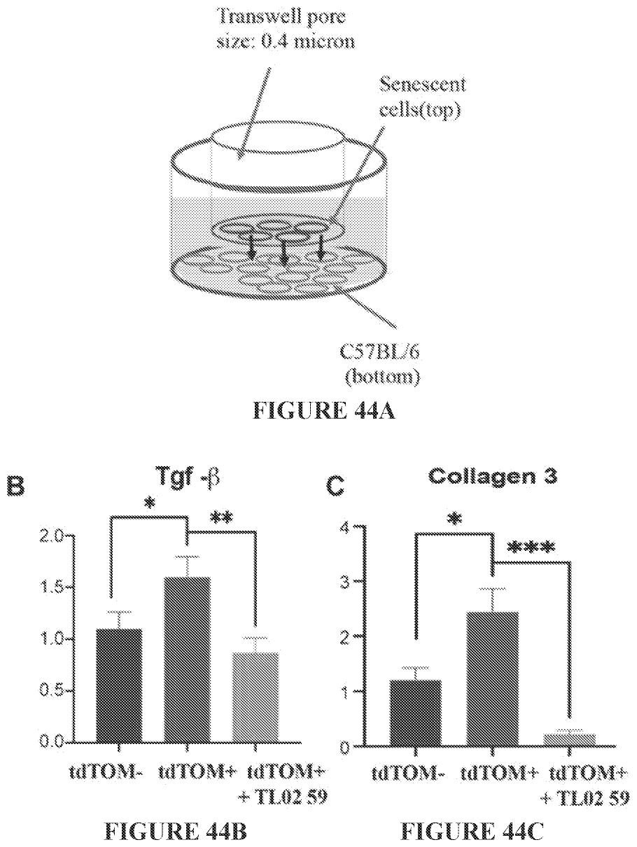

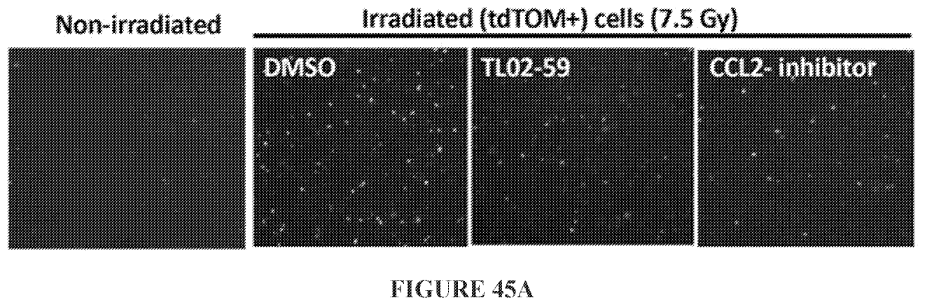

Fgr inhibitor TL02-59 inhibits the senescence cell mediated induction of fibrosis biomarkers in target cells in transwell cultures. To determine the correlation of senescent cells in the lungs with fibrosis to the lungs, an in vitro transwell culture system was used. As shown in FIG. 44A-FIG. 44C, radiation-induced (5 Gy, 10 days) tdTOMp16+ bone marrow senescent cells were purified by FACS and were plated at the top well of transwell cultures. The senescent cells in the top chamber of transwell cultures that were separated by a 0.3-micron filter (FIG. 44A), induced profibrotic genes in target (mesenchymal stem cells) cells in the bottom chamber of transwell cultures after 10 days including: TFG-β and Collagen 3 (FIG. 44B and FIG. 44C, respectively). The induction of these biomarkers of fibrosis was blocked by adding to the transwell cultures the small molecule inhibitor of Fgr kinase TL02-59 (FIG. 44B and FIG. 44C).

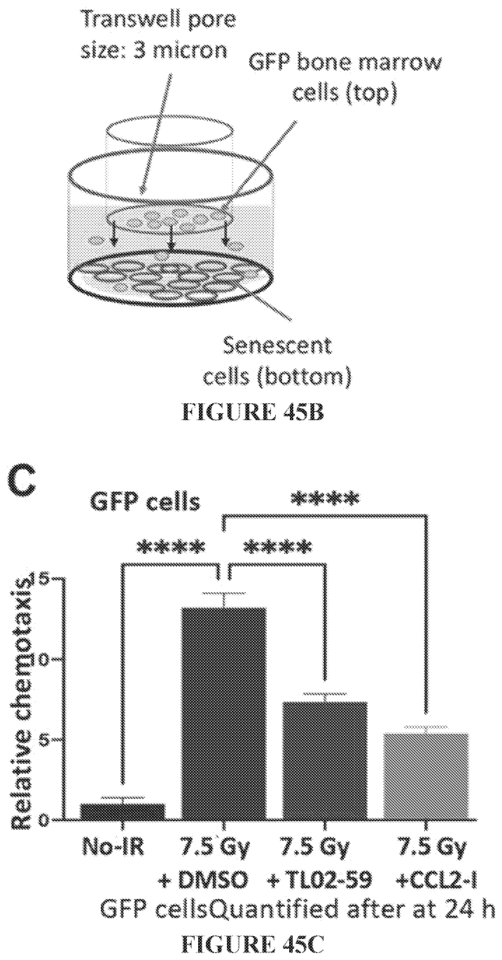

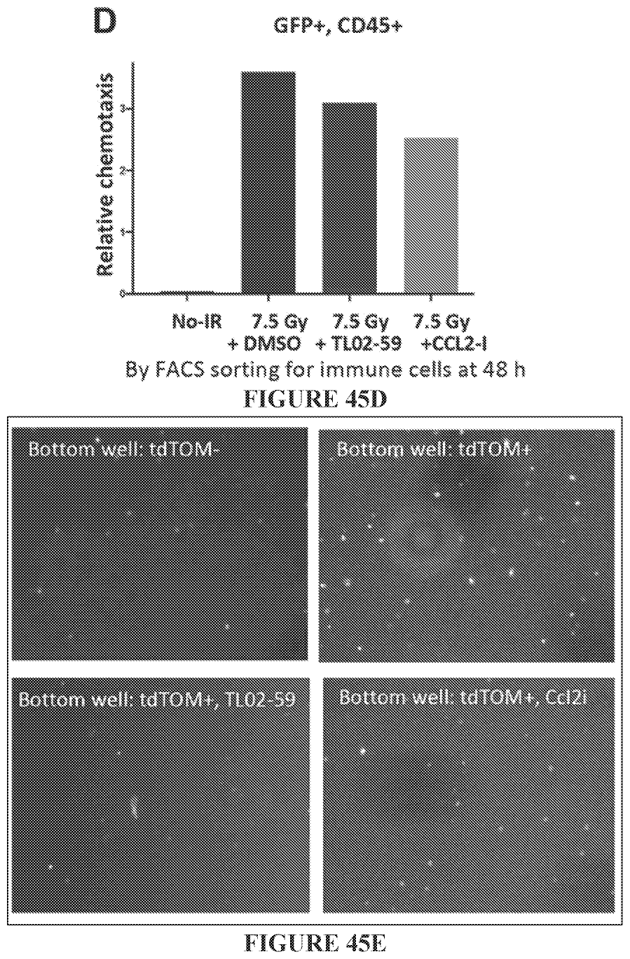

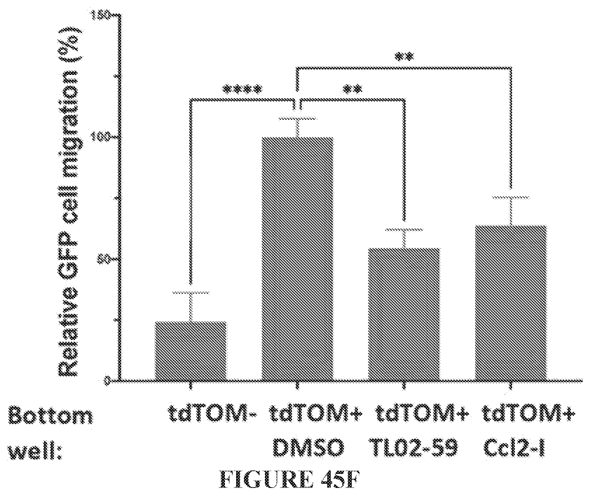

Inhibition of tyrosine kinase Fgr by TL02-59 inhibits migration of bone marrow cells towards senescent cells. In a transwell coculture system, the role of Fgr in the migration of GFP bone marrow cells across the 3-micron filter towards the senescent cells was evaluated next (FIG. 45B). To determine whether inhibition of tyrosine kinase Fgr in senescent cells blocks the bone marrow migration of gfp+ monocyte/macrophages towards the senescent cells at the bottom wells, the Fgr tyrosine kinase inhibitor TL02-59 (10 nM) was added in the media and the migrated cells were evaluated after 48 h of TL02-59 treatment. TL02-59 significantly reduced the migration of gfp+ monocyte/macrophages towards the senescent cells isolated from irradiated tdTOMp16+ stromal cell line (FIG. 45A, FIG. 45C, and FIG. 45D). Similar results were observed when senescent cells isolated from the tdTOMp16+ lungs 150-days post radiation were used (FIG. 45E and FIG. 45F). It was confirmed that the migrating cells were monocyte/macrophages by adding a monocyte chemotaxis inhibitor, which is a Ccl2 receptor antagonist (FIG. 45A and FIG. 45E). The present results establish that Fgr positive senescent cells recruit the gfp+ bone marrow derived monocyte/macrophage precursors that are separated by a 3-micron filter in a transwell culture system, and that the migration is blocked by a small molecule, which inhibits Fgr, TL02-59.

Discussion. The sequence of cellular events occurring in the lung during evolution of silicosis and the phenotype(s) of cells that are induced at each stage are not known. In the present study, the appearance of senescent cells, and, specifically, monocyte/macrophages and neutrophils in the lungs of mice injected with crystalline silica were the focus. The findings were corroborated in the radiation-induced pulmonary fibrosis model. The availability of mouse strains were taken advantage of, which can be used to demonstrate the link between the occurrence of senescence and upregulation of p16+, a specific biological marker for senescence. The p16^{+/LUC} mice (Liu J Y et al. Proc Natl Acad Sci U.S.A. 116(7): 2603-2611, 2019), which can be imaged serially by the IMUS Imaging System, demonstrated senescence-associated p16-linked luciferase activity in the presence of luciferin as early as day 7 after crystalline silica injection. These areas of senescence increased in magnitude through day 28, when florid fibrosis is detected. Explants of the lungs from these mice as well as control C57BL/6J and tdTOMp16+ (Sessions G A et al. FASEB J 33(11): 12364-12373, 2019) mice confirmed the presence of senescent cells in the lungs at the same time points, as the p16^{+/LUC} imaging results.

The appearance of specific phenotypes of senescent cells in the irradiated lungs of mice has been reported previously (Epperly M W et al. Radiat Res 196(3): 235-249, 2021). The present study extends these findings regarding the kinetics of appearance of senescent cells in the lungs after intratracheal injection of crystalline silica in mice. Senescent cells in the lungs were demonstrated to be primarily monocytes/macrophages. Furthermore, bone marrow derived monocytes/macrophages were demonstrated in the lungs of gfp+ marrow chimeric mice that had developing silicosis. Increasing numbers of resident (recipient origin) pulmonary epithelial cells and monocytes/macrophages were observed to be senescent. After crystalline silica intratracheal injection, gfp+ bone marrow chimeric tdTOMp16+ mice demonstrated significant bone marrow derived monocytes/macrophages in the lungs, and these cells were positive for the senescence-associated Fgr tyrosine kinase as well as other biomarkers of senescence including p16.

To confirm that crystalline silica-injected senescent mouse lung cells were recruiting bone marrow derived monocytes/macrophages, a transwell culture system in which senescent cells on the bottom were separated from gfp+ bone marrow cells in the top of the transwell separated by 3-micron filter was established. In these experiments, radiation-induced senescent cells were used, which recruited gfp+ monocytes/macrophages through the filter, and these bone marrow derived cells accumulated on the bottom surface of the filter. Migration of gfp+ bone marrow cells in response to senescent cells was inhibited by the small molecule Fgr inhibitor TL02-59. The role of Fgr in senescent cells and in pulmonary fibrosis has been a subject of recent interest. Tyrosine kinases are involved in many inflammatory and fibrotic processes (Lowell C A. Mol Immunol 41(6-7): 631-643, 2004; Okutani D et al. Am J Physiol Lung Cell Mol Physiol 291(2): L129-L141, 2006; Meng F et al. J Exp Med 185(9): 1661-1670, 1997; Acin-Pérez R et al. Nat Metab 2(9): 974-988, 2020; Jing X et al. Acta Pharm Sin B 11(2): 394-405, 2021; Lowell C A et al. J Cell Biol 133(4): 895-910, 1996; KovAcs M et al. J Exp Med 211(10): 1993-2011, 2014; Ingley E. Biochim Biophys Acta 1784(1): 56-65, 2008). Using transwell cultures, it was shown that inhibition of Fgr reduced their capacity to induce biomarkers of fibrosis in target cells and to induce chemotaxis of gfp+ marrow origin cells. It was also demonstrated that areas of fibrosis were present in the lungs of crystalline silica-injected mice juxtaposed to the areas that showed p16 positive senescent epithelial cells. Interestingly, single cell suspensions from whole lungs revealed that senescent cells were predominantly of the monocyte/macrophage phenotype and a significant percentage were of bone marrow origin. The sequence and time course of these cellular events is currently being studied.

The present data establish that cells of bone marrow origin are a component of the monocyte/macrophage and neutrophil response in the lungs during the development of silicosis. They also document the appearance of senescent cells in the lungs in areas of fibrosis. Further studies can elucidate clearly and specifically the interaction between pulmonary epithelial cells, resident alveolar and interstitial macrophages with bone marrow derived migrating monocyte/macrophages, and the cell phenotype of those cells accumulating alpha-smooth muscle actin and proliferating fibroblasts to form fibrosis.

### Example 7

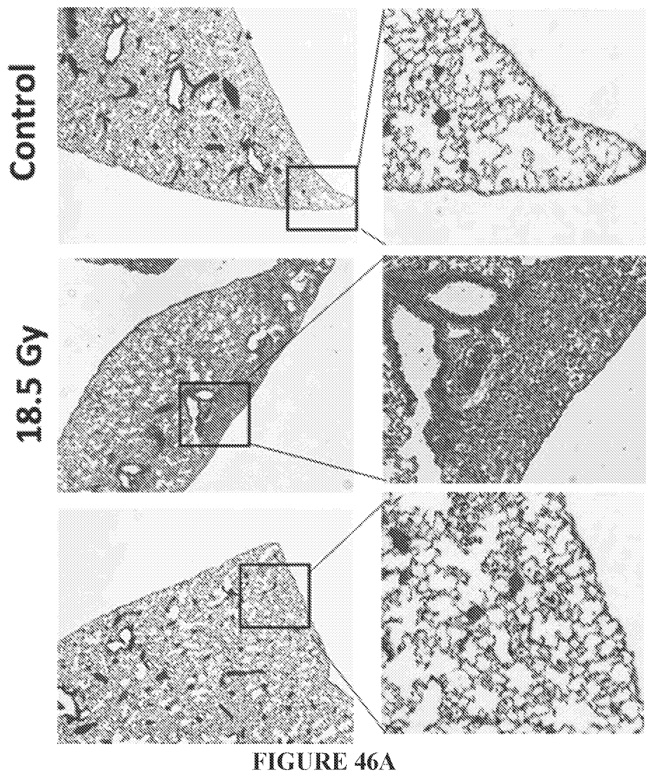

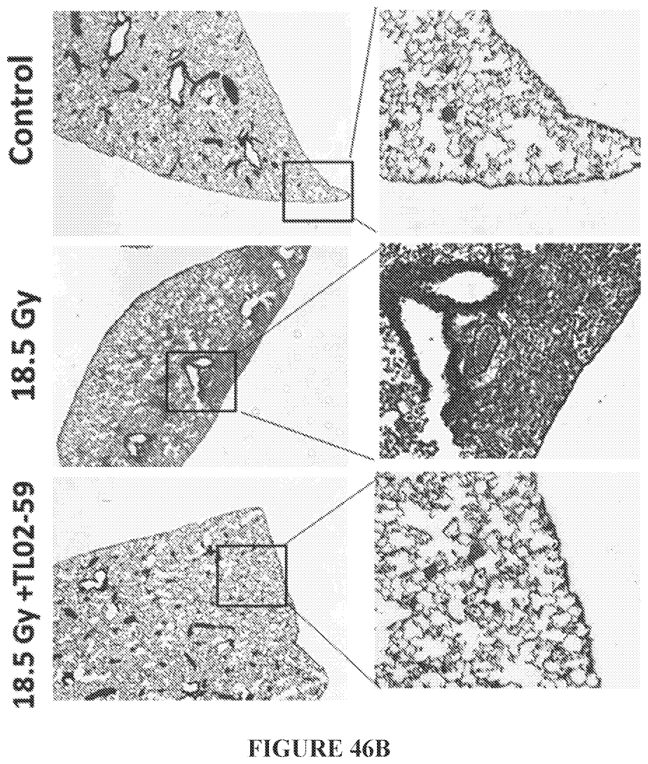

FIG. 46A and FIG. 46B. Representative images of control (top row panels), irradiated (18 Gy) (middle row panels), and irradiated plus TL02-59 treated (bottom row panels) mouse lungs stained using H&E (FIG. 46A) or Trichrome (FIG. 46B).

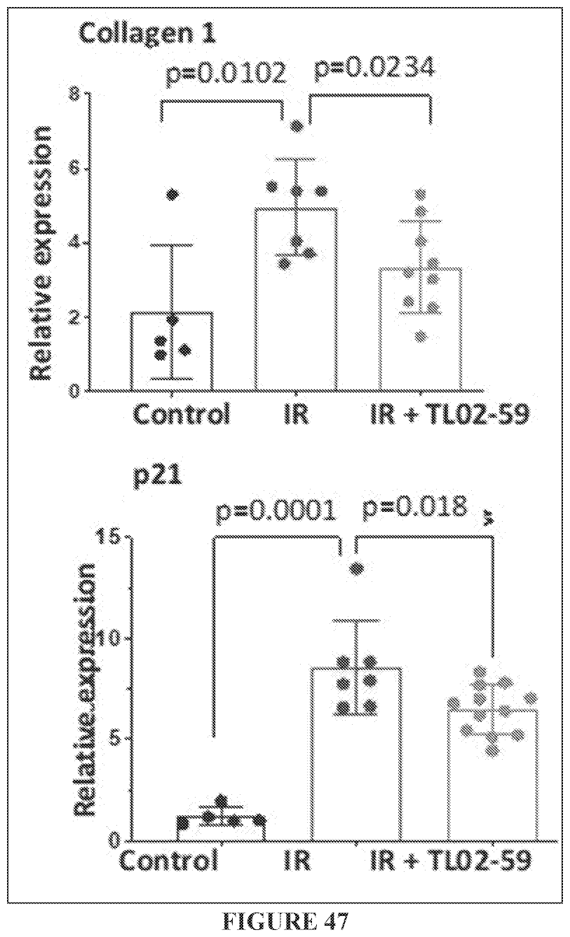

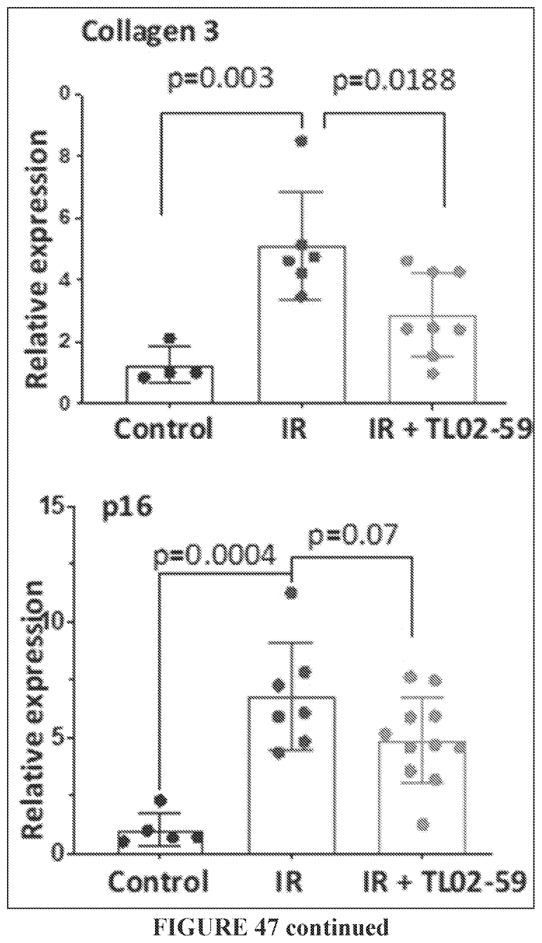

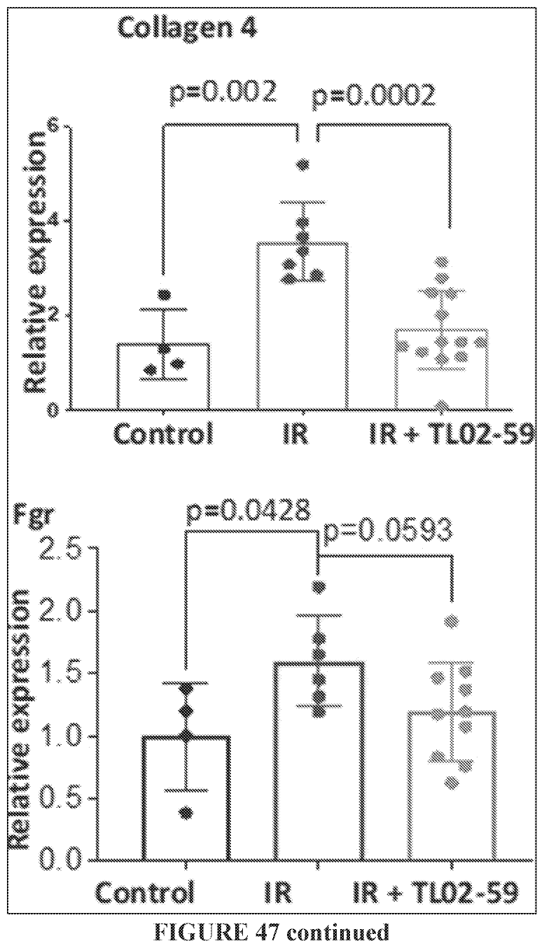

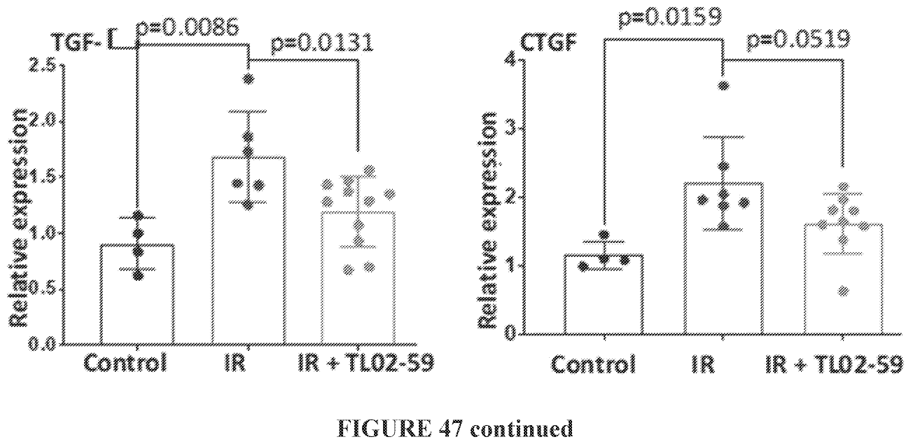

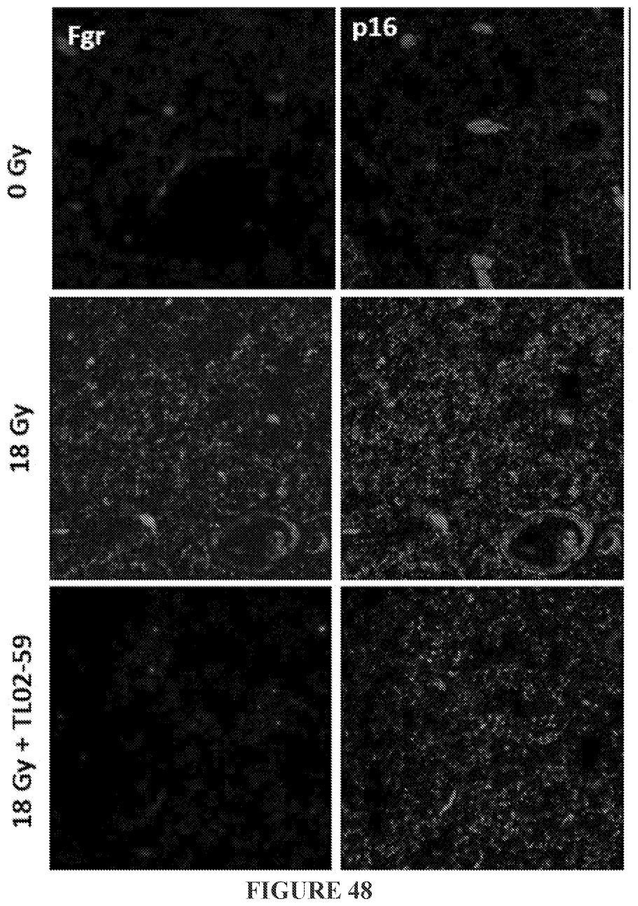

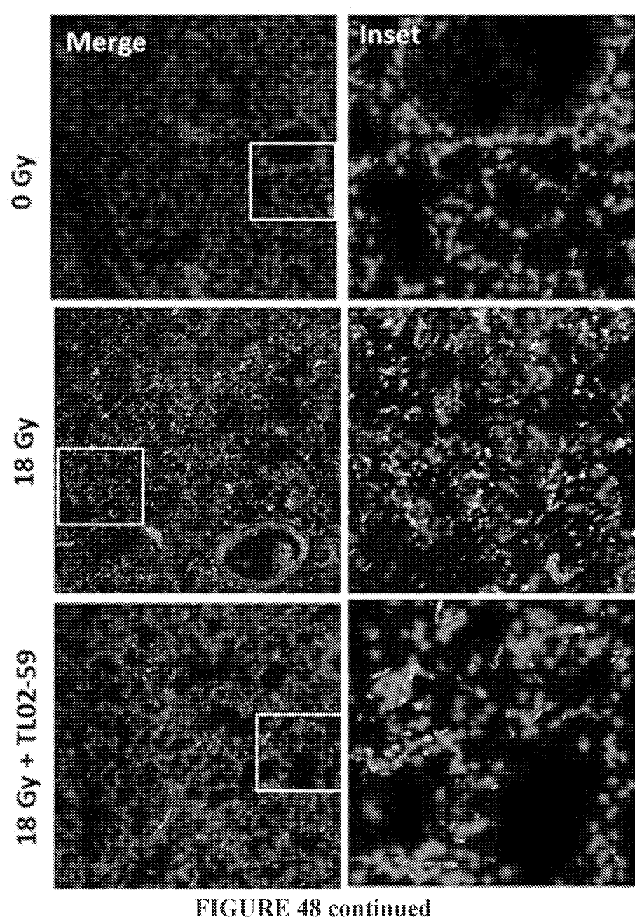

FIG. 47. Fgr inhibitor TL02-59 treatment reduces profibrotic and biomarkers of senescence in a mouse model of radiation-induced pulmonary fibrosis. Relative mRNA expression of profibrotic genes (Tgf-β, Ctgf, Collagen 1a1, Collagen 3 and Collagen 4), senescence biomarkers (p16 and p21) and tyrosine kinase Fgr was evaluated by qPCR from one lobe of mouse lungs in control, irradiated and treated with Fgr inhibitor TL02-59 post irradiation. Briefly, Mice were irradiated (18 Gy), and TL02-59 treated (10 mg/kg), 3/week at day 75 for 4 weeks. (n=4-13), Statistics was performed, and individual p values were calculated by unpaired t-test.

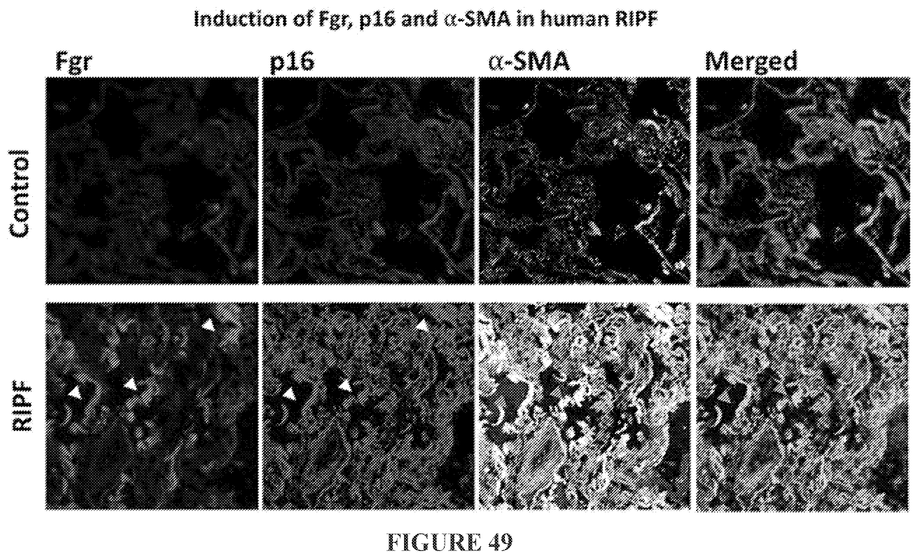

FIG. 48. Expression of Fgr and p16 from mouse lungs in control, irradiated, and treated with Fgr inhibitor TL02-59 post irradiation. Images show induction of Fgr and senescence marker Fgr upon irradiation and TL02-59 treatment led to a reduction of both the proteins. Insets on the right from each group were magnified for clarity.

FIG. 49. Induction of Fgr, p16, and α-SMA in cells within human RIPF. Surgically resected lung of a radiotherapy patient at 24-months after 60 Gy treatment for lung cancer, and age matched control lungs were stained for Fgr, p16, α-SMA and nuclear stain DAPI (20×). Images are representative of 3 separate patient samples.

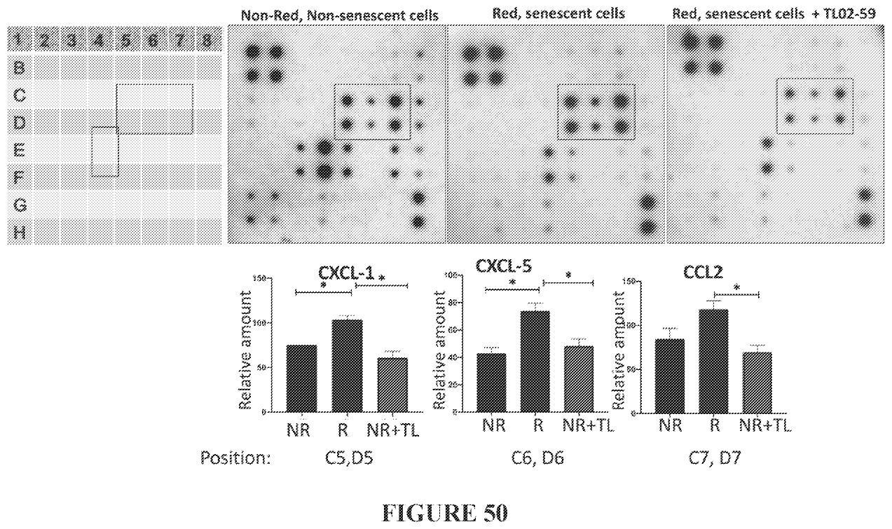

FIG. 50. Fgr inhibitor TL02-59 reduces secretion of senescence associated secretory chemokines. tdTOMp16 bone marrow stromal cells were irradiated (5 Gy) and after 10 days tdTOMp16+ red senescent cells and non-red non-senescent were FACS sorted and cultured with TL02-59 (10 nM) for 72 hours. Using chemokine array relative amounts of chemokines secreted in the media was measured. (n=2, *, p<0.05, p values were calculated using unpaired t-test).

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method for treating or preventing radiation-induced fibrosis in a subject in need thereof, the method comprising: administering to the subject an effective amount of a composition that inhibits Fgr (a non-receptor tyrosine kinase).

2. The method of claim 1, wherein the radiation-induced fibrosis is an ionizing radiation-induced fibrosis.

3. The method of claim 1, wherein the composition that inhibits Fgr is administered to the subject:
prior to exposure of the subject to ionizing radiation;
during exposure of the subject to ionizing radiation; or
after exposure of the subject to ionizing radiation.

4. (canceled)

5. (canceled)

6. (canceled)

7. The method of claim 1, wherein the composition that inhibits Fgr is a N-phenylbenzamide tyrosine kinase inhibitor.

8. The method of claim 1, wherein the composition that inhibits Fgr comprises shRNA that interferes with Fgr expression; CRISPR for editing or knockout of Fgr; siRNA, antisense RNA, or nucleic acids that interfere with or prevent the transcription or translation of Fgr genes; antisense molecules comprising Fgr sequences; active sites that bind at least a portion of Fgr; interfering peptides; interfering nucleic acids; or a combination thereof.

9. The method of claim 8, wherein the composition that inhibits Fgr comprises shRNA that interferes with Fgr expression.

10. The method of claim 1, wherein the composition that inhibits Fgr comprises MMS350, TL02-59, or a combination thereof.

11. The method of claim 17, wherein the disease or condition is mediated by Fgr.

12. (canceled)

13. (canceled)

14. The method of claim 17, wherein the composition that inhibits Fgr is TL02-59.

15. The method of claim 17, wherein the composition that inhibits Fgr comprises shRNA that interferes with Fgr expression; CRISPR for editing or knockout of Fgr; siRNA, antisense RNA, or nucleic acids that interfere with or prevent the transcription or translation of Fgr genes; antisense molecules comprising Fgr sequences; active sites that bind at least a portion of Fgr; interfering peptides; interfering nucleic acids; or a combination thereof.

16. (canceled)

17. A method for treating or preventing a disease or condition characterized by aberrant fibroblast proliferation and extracellular matrix deposition in a tissue of a subject in need thereof, the method comprising: administering to the subject an effective amount of a composition that inhibits Fgr.

18. The method of claim 17, wherein the disease or condition result from idiopathic pulmonary fibrosis (IPF), pneumonia, acute respiratory distress syndrome (ARDS), asbestosis, bleomycin exposure, silicosis, anthracosis, post bacterial infectious fibrosis, viral (including Covid-19) liver fibrosis, post heat burn fibrosis, post ultraviolet light fibrosis, post trauma fibrosis, myocardial infarction, injury related tissue scarring, scarring form surgery, radiation exposure, allergic reaction, inhalation of environmental particulates, smoking, infection, mechanical damage, transplantation, autoimmune disorder, genetic disorder, a disease condition (such as scleroderma lung disease, rheumatoid arthritis, sarcoidosis, tuberculosis, Hermansky Pudlak Syndrome, bagassosis, systemic lupus erythematosis, eosinophilic granuloma, Wegener's granulomatosis, lymphangioleiomyomatosis, or cystic fibrosis), nitrofurantoin exposure, amiodarone exposure, cyclophosphamide exposure, methotrexate exposure, or a combination thereof.

19. The method of claim 17, wherein the disease or condition is fibrosis.

20. (canceled)

21. The method of claim 19, wherein the fibrosis is lung fibrosis.

22. The method of claim 19, wherein the fibrosis is radiation-induced fibrosis.

23. (canceled)

24. The method of claim 17, wherein the composition that inhibits Fgr is a N-phenylbenzamide tyrosine kinase inhibitor.

25. The method of claim 17, wherein the composition that inhibits Fgr comprises shRNA that interferes with Fgr expression; CRISPR for editing or knockout of Fgr; siRNA, antisense RNA, or nucleic acids that interfere with or prevent the transcription or translation of Fgr genes; antisense molecules comprising Fgr sequences; active sites that bind at least a portion of Fgr; interfering peptides; interfering nucleic acids; or a combination thereof.

26. The method of claim 17, wherein the composition that inhibits Fgr comprises MMS350, TL02-59, or a combination thereof.

27. The method of claim 1, the method further comprising irradiating the subject with ionizing radiation.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. A method of identifying a pharmacological compound for treating or preventing fibrosis in a subject in need thereof, the method comprising:
a) obtaining a population of p16 or β-galactosidase (SA-β-gal) expressing senescent cells from the subject;
b) identifying Fgr positive senescent cells;
c) contacting the Fgr positive senescent cells with the pharmacological compound; and
d) determining whether the pharmacological compound inhibits Fgr in the Fgr positive senescent cells.

41. (canceled)

42. (canceled)

43. (canceled)

44. (canceled)

45. (canceled)

46. (canceled)

47. (canceled)
